(19) United States
(12) Patent Application Publication
(10) Pub. No.: US 2020/0206269 A1
(43) Pub. Date: Jul. 2, 2020

(54) PRODUCTION OF ENUCLEATED RED BLOOD CELLS AND USES THEREOF

(71) Applicants: Whitehead Institute for Biomedical Research, Cambridge, MA (US); Trustees of Tufts College, Medford, MA (US)

(72) Inventors: Harvey Lodish, Brookline, MA (US); Nai-Jia Huang, Somerville, MA (US); Novalia Pishesha, Cambridge, MA (US); Hidde L. Ploegh, Boston, MA (US); Charles Shoemaker, North Grafton, MA (US)

(73) Assignees: Whitehead Institute for Biomedical Research, Cambridge, MA (US); Trustees of Tufts College, Medford, MA (US)

(21) Appl. No.: 16/641,203

(22) PCT Filed: Aug. 22, 2018
(86) PCT No.: PCT/US2018/047575

§ 371 Date: Feb. 21, 2020

Related U.S. Application Data
(60) Provisional application No. 62/549,373, filed on Aug. 23, 2017.

(51) Int. Cl. A61K 35/18; C07K 16/12; C07K 19/00; C12N 5/0789; C12N 15/86

(52) U.S. Cl. CPC A61K 35/18; C12N 2740/15043; C07K 19/00; C12N 5/0647; C12N 15/86; C07K 2317/34; C07K 2319/03; C07K 2317/569; C12N 2500/25; C12N 2500/30; C12N 2501/91; C12N 2501/33; C12N 2501/26; C12N 2501/125; C12N 2501/2303; C12N 2501/2306; C12N 2501/14; C07K 16/1282

(57) ABSTRACT

Multi-step methods for the in vitro production of enucleated red blood cells and the enucleated red blood cells thus prepared are provided. Such enucleated red blood cells may express fusion proteins comprising an antigen binding protein which allows the red blood cell to bind a toxin or an antigen of a pathogen. Also described herein are methods for neutralizing a toxin or pathogen in a subject by administering enucleated red blood cells that express any of the fusion proteins provided herein.

### RELATED APPLICATIONS

This application claims priority to U.S. Provisional Application No. 62/549,373, filed on Aug. 23, 2017, which is incorporated by reference herein.

### GOVERNMENT SUPPORT

This invention was made with government support under Grant No. HR0011-14-2-0005, awarded by the Defense Advanced Research Projects Agency (DARPA). The government has certain rights in the invention.

### BACKGROUND OF THE INVENTION

VHHs are single domain antibodies of molecular weight ˜15 kD that are derived from the unusual heavy-chain-only antibodies produced by camelids¹. Compared to conventional antibodies, VHHs are more stable and are typically better expressed in recombinant hosts. They also have a greater tendency to recognize conformational shapes (reviewed in²). While single VHHs can be potent toxin neutralizing agents, greatly improved therapeutic efficacy has been demonstrated in several animal models when two or more different toxin neutralizing VHHs were linked and expressed as multi-specific VHH-based neutralizing agents (VNAs)^{3, 4, 5, 6, 7}.

### SUMMARY OF THE INVENTION

The present disclosure is based, at least in part, on the development of an in vitro multi-phase culturing process for differentiating human CD34⁺ peripheral blood cells into red blood cells or enucleated precursors thereof. It was surprisingly discovered that the culturing process provided herein yielded a 3 fold improvement in the extent of enucleation (more than 90% enucleation) and a 2 fold increase in cell yield as compared to previously described culturing processes. See Lee H Y, et al. PPAR-alpha and glucocorticoid receptor synergize to promote erythroid progenitor self-renewal. Nature 522, 474-477 (2015); and Griffiths R E, et al. Maturing reticulocytes internalize plasma membrane in glycophorin A-containing vesicles that fuse with autophagosomes before exocytosis. Blood 119, 6296-6306 (2012). The present disclosure is also based on the surprising discovery that red blood cells engineered to express fusion proteins comprising a red blood cell transmembrane protein fused to one or more VHH domains (e.g., botulinum toxin-binding VHH domain(s)), effectively protected against botulinum toxin lethality in vivo. Engineered enucleated red blood cells can be produced using the in vitro multi-phase culturing systems described herein. Such modified enucleated red blood cells can be used for therapeutic purposes, for example, neutralizing toxins or pathogens in a subject.

Accordingly, some aspects of the present disclosure feature a multi-step method for producing CD34⁺ human progenitor cells. In one aspect the disclosure provides a method for producing human red blood cells or enucleated precursors thereof, the method comprising the following steps: (i) providing a population of human CD34⁺ progenitor cells; (ii) expanding the population of human CD34⁺ progenitor cells in a expansion medium for 1 to 6 days, wherein the expansion medium comprises Flt-3 ligand, stem cell factor (SCF), interleukin 3 (IL-3), and interleukin 6 (IL-6); (iii) differentiating the population of human CD34⁺ progenitor cells expanded in (ii) in a first differentiation medium for 2 to 6 days, wherein the first differentiation medium comprises SCF, IL-3, insulin, erythropoietin (EPO), and holo-transferrin;
(iv) differentiating the population of human CD34⁺ progenitor cells from (iii) in the first differentiation medium for 1 to 5 days,
(v) differentiating the population of human CD34⁺ progenitor cells from (iv) in a second differentiation medium for 2 to 6 days, wherein the second differentiation medium comprises SCF, insulin, EPO, and holo-transferrin;
(vi) differentiating the population of human CD34⁺ progenitor cells from (v) in a third differentiation medium for 2 to 6 days, wherein the third differentiation medium comprises insulin, EPO, and holo-transferrin; and
(vii) differentiating the population of human CD34⁺ progenitor cells from (vi) in a fourth differentiation medium for 1 to 4 days, wherein the fourth differentiation medium comprises insulin, and holo-transferrin, thereby producing human red blood cells or enucleated precursors thereof.

In some embodiments, the expansion medium comprises Flt-3 ligand at a concentration of 50 ng/ml to 200 ng/ml, SCF at a concentration of 50 ng/ml to 200 ng/ml, IL-3 at a concentration of 10 ng/ml to 40 ng/ml; and IL-6 at a concentration of 10 ng/ml to 40 ng/ml. In some embodiments, the expansion medium further comprises Stemspan II medium. In some embodiments, the expansion medium further comprises dexamethasone. In some embodiments, the expansion medium comprises dexamethasone at a concentration of 50 nM to 200 nM.

In some embodiments, the human CD34⁺ progenitor cells in step (ii) are cultured at an initial cell density from 250,000 to 1,500,000 cells/mL. In some embodiments, the human CD34⁺ progenitor cells in step (ii) are cultured at an initial cell density of 500,000 cells/ml. In some embodiments, the human CD34⁺ progenitor cells in step (ii) are expanded for 5 days.

In some embodiments, the first differentiation medium comprises SCF at a concentration of 5 ng/ml to 20 ng/ml, IL-3 at a concentration of 0.5 ng/ml to 2 ng/ml, insulin at a concentration of 5 μg/ml to 20 μg/ml; EPO at a concentration of 0.1 U/ml to 6 U/ml and holo-transferrin at a concentration of 100 μg/ml to 400 μg/ml. In some embodiments, the first differentiation medium further comprises IMDM medium. In some embodiments, the first differentiation medium further comprises heparin. In some embodiments, the first differentiation medium comprises heparin at a concentration of 1.5 U/ml to 6 U/ml. In some embodiments, first differentiation medium further comprises human blood plasma. In some embodiments, the first differentiation medium comprises from 1% to 4% human blood plasma. In some embodiments, the first differentiation medium further comprises human serum. In some embodiments, the first differentiation medium comprises from 1.5% to 6% human serum.

In some embodiments, the human CD34⁺ progenitor cells in step (iii) are maintained at a density from 50,000 to 400,000 cells/mL. In some embodiments, the density of the human CD34⁺ progenitor cells in step (iii) is 100,000 cells/ml. In some embodiments, the human CD34⁺ progenitor cells in step (iii) are differentiated for 4 days.

In some embodiments, the human CD34⁺ progenitor cells in step (iv) are maintained at a density from 100,000 to 600,000 cells/mL. In some embodiments, the density of the human CD34⁺ progenitor cells in step (iv) is 400,000 cells/ml. In some embodiments, the human CD34⁺ progenitor cells in step (iv) are differentiated for 3 days.

In some embodiments, the second differentiation medium comprises SCF at a concentration of 5 ng/ml to 20 ng/ml, insulin at a concentration of 5 μg/ml to 20 μg/ml; EPO at a concentration of 0.05 U/ml to 4 U/ml and holo-transferrin at a concentration of 100 μg/ml to 400 μg/ml. In some embodiments, the second differentiation medium further comprises IMDM medium. In some embodiments, the second differentiation medium further comprises heparin. In some embodiments, the second differentiation medium comprises heparin at a concentration of 1.5 U/ml to 6 U/ml. In some embodiments, the second differentiation medium further comprises human blood plasma. In some embodiments, the second differentiation medium comprises from 1% to 4% human blood plasma. In some embodiments, the second differentiation medium further comprises human serum. In some embodiments, the second differentiation medium comprises from 1.5% to 6% human serum.

In some embodiments, the human CD34⁺ progenitor cells in step (v) are maintained at a density from 50,000 to 400,000 cells/mL. In some embodiments, the density of the human CD34⁺ progenitor cells in step (v) is 100,000 cells/ml. In some embodiments, the human CD34⁺ progenitor cells in step (v) are differentiated for 4 days.

In some embodiments, the third differentiation medium comprises insulin at a concentration of 5 μg/ml to 20 μg/ml; EPO at a concentration of 0.01 U/ml to 0.2 U/ml and holo-transferrin at a concentration of 250 μg/ml to 1000 μg/ml. In some embodiments, the third differentiation medium further comprises IMDM medium. In some embodiments, the third differentiation medium further comprises heparin. In some embodiments, the third differentiation medium comprises heparin at a concentration of 1.5 U/ml to 6 U/ml. In some embodiments, the third differentiation medium further comprises human blood plasma. In some embodiments, the third differentiation medium comprises from 1% to 4% human blood plasma. In some embodiments, the third differentiation medium further comprises human serum. In some embodiments, the third differentiation medium comprises from 1.5% to 6% human serum.

In some embodiments, the human CD34⁺ progenitor cells in step (vi) are maintained at a density from 50,000 to 200,000 cells/mL. In some embodiments, the density of the human CD34⁺ progenitor cells in step (vi) is 100,000 cells/ml. In some embodiments, the human CD34⁺ progenitor cells in step (vi) are differentiated for 4 days.

In some embodiments, the fourth differentiation medium comprises insulin at a concentration of 5 μg/ml to 20 μg/ml; and holo-transferrin at a concentration of 250 μg/ml to 1000 μg/ml. In some embodiments, the fourth differentiation medium further comprises IMDM medium. In some embodiments, the fourth differentiation medium further comprises heparin. In some embodiments, the fourth differentiation medium comprises heparin at a concentration of 1.5 U/ml to 6 U/ml. In some embodiments, the fourth differentiation medium further comprises human blood plasma. In some embodiments, the fourth differentiation medium comprises from 1% to 4% human blood plasma. In some embodiments, the fourth differentiation medium further comprises human serum. In some embodiments, the fourth differentiation medium comprises from 1.5% to 6% human serum.

In some embodiments, the human CD34⁺ progenitor cells in step (vii) are increased to a density from 2,500,000 to 10,000,000 cells/mL. In some embodiments, the human CD34⁺ progenitor cells in step (vii) are increased to a density of 5,000,000 cells/mL. In some embodiments, the human CD34⁺ progenitor cells in step (vii) are differentiated for 3 days.

In some aspects the disclosure provides a method for producing human red blood cells or enucleated precursors thereof (e.g., reticulocytes), the method comprising:

maintaining a plurality of nucleated red blood cell precursors under conditions (maturation conditions) that allow for maturation of a plurality of the precursor cells into red blood cells or enucleated precursors thereof, wherein:

a) the level of EPO or an EPO analog under the maturation conditions is less than 1, 0.5, 0.3 or 0.1 units/ml, e.g., EPO or an EPO analog is absent;

b) the maturation conditions comprise adding no EPO or EPO analog or less than 1, 0.5, 0.3 or 0.1 units/ml of the EPO or EPO analog to the medium (e.g., to the maturation medium) in which the cells are matured; or

c) the level of EPO or EPO analog under the maturation conditions is lower than an amount of EPO in a differentiation medium (e.g., differentiation medium IV) that the cells have been previously cultured (e.g., immediately prior to) in, e.g., decreased by up to 5%, 10%, 15%, 20%, 30%, 40%, 50%, 60%, 70%, 80%, 90%, 95%, 99%, or 100%, or decreased by up to 0.01 U/ml, 0.02 U/ml, 0.03 U/ml, 0.04 U/ml, 0.05 U/ml, 0.06 U/ml, 0.07 U/ml, 0.08 U/ml, 0.09 U/ml, 0.1 U/ml, 0.15 U/ml, 0.2 U/ml, 0.25 U/ml, 0.3 U/ml, 0.4 U/ml, 0.5 U/ml, 0.6 U/ml, 0.7 U/ml, 0.8 U/ml, 1.0 U/ml, 1.5 U/ml, or 2.0 U/ml;

thereby providing a population comprising red blood cells or enucleated precursors thereof or a combination thereof,

wherein at least 70%, 75%, 80%, 85%, 90%, or 95% of the cells in the population are red blood cells or enucleated precursors thereof or a combination thereof.

In some embodiments, the EPO analog is chosen from Epoetin alfa, Epoetin beta, or Darbepoetin alfa. In some embodiments, the EPO is from a horse, pig, rabbit, goat, cow or human.

In some embodiments, the method does not comprise adding EPO or an EPO analog to the cells.

In some embodiments, the maturation conditions further comprise insulin (e.g., from 5 μg/ml to 20 μg/ml insulin), holo transferrin (e.g., from 400 μg/ml to 600 μg/ml holo human transferrin), or both of insulin and holo transferrin.

In some embodiments, the method further comprises culturing nucleated red blood cell precursor cells in one or more of differentiation medium I, differentiation medium II, differentiation medium III, differentiation medium IV prior to maturation conditions. In some embodiments, the differentiation medium in which the cells were previously cultured (e.g., immediately prior to maturation conditions) comprises EPO or an EPO analog at a level of from 0.01 U/ml to 0.05 U/ml, from 0.01 U/ml to 0.1 U/ml, from 0.01 U/ml to 0.2 U/ml, from 0.01 U/ml to 0.5 U/ml, from 0.01 U/ml to 1.0 U/ml, from 0.05 U/ml to 0.1 U/ml, from 0.05 U/ml to 0.2 U/ml, from 0.05 U/ml to 0.5 U/ml, from 0.05 U/ml to 1.0 U/ml, from 0.1 U/ml to 0.2 U/ml, from 0.1 U/ml to 0.5 U/ml, from 0.1 U/ml to 1.0 U/ml, from 0.2 U/ml to 0.5 U/ml, from 0.2 U/ml to 1.0 U/ml, or from 0.5 U/ml to 1.0 U/ml. In some embodiments, the differentiation medium in which the cells were previously cultured (e.g., immediately prior to maturation conditions) comprises EPO or an EPO analog at a level of from 0.1 U/ml-10 U/ml (e.g., 0.1-8 U/ml, 0.1-5 U/ml, 0.5-2 U/ml, or 0.8-1.2 U/ml). In some embodiments, the amount of EPO in the medium ranges from 0.1 U/ml to 0.5 U/ml, from 0.1 U/ml to 1 U/ml, from 0.1 U/ml to 2 U/ml, from 0.1 U/ml to 5 U/ml, from 0.1 U/ml to 10 U/ml, from 0.5 U/ml to 1 U/ml, from 0.5 U/ml to 2 U/ml, from 0.5 U/ml to 5 U/ml, from 0.5 U/ml to 10 U/ml, from 1 U/ml to 2 U/ml, from 1 U/ml to 5 U/ml, from 1 U/ml to 10 U/ml, from 2 U/ml to 5 U/ml, from 2 U/ml to 10 U/ml, or from 5 U/ml to 10 U/ml.

In some embodiments, the density of nucleated red blood cell precursors is from 1×10³ to 1×10⁴ cells/mL, from 1×10³ to 1×10⁵ cells/mL, from 1×10³ to 1×10⁶ cells/mL, from 1×10³ to 1×10⁷ cells/mL, from 1×10⁴ to 1×10⁵ cells/mL, from 1×10⁴ to 1×10⁶ cells/mL, from 1×10⁴ to 1×10⁷ cells/mL, from 1×10⁵ to 1×10⁶ cells/mL, from 1×10⁵ to 1×10⁷ cells/mL, or from 1×10⁶ to 1×10⁷ cells/mL. In some embodiments, the density of nucleated red blood cell precursors is from about 2,000,000 cells/mL to about 8,000,000 cells/mL (e.g., about 5,000,000 cells/mL). In some embodiments, the density of nucleated red blood cell precursors under maturation conditions is greater than the density of cells cultured (e.g., immediately prior to) in a previous differentiation medium (e.g., differentiation medium IV). In some embodiments, the density of nucleated red blood cell precursors under maturation conditions is increased by up to 50%, 100%, 150%, 200%, 300%, 400%, 500%, 600%, 700%, 800%, 900%, 1000%, 1500%, 2000%, 2500%, 3000%, 4000%, or 5000% as compared to the density of cells cultured (e.g., immediately prior to) in a previous differentiation medium (e.g., differentiation medium IV). In some embodiments, the density of nucleated red blood cell precursors under maturation conditions is increased up to 500,000 cells/mL, 1,000,000 cells/mL, 2,000,000 cells/mL, 4,000,000 cells/mL, 5,000,000 cells/mL, or 6,000,000 cells/mL, 7,000,000 cells/mL, 8,000,000 cells/mL, or 9,000,000 cells/mL as compared to the density of cells cultured (e.g., immediately prior to) in a previous differentiation medium (e.g., differentiation medium IV).

In some embodiments, one or more of the nucleated red blood cell precursors comprises a vector, e.g., a vector encoding a protein of interest.

In certain aspects, the disclosure comprises a preparation comprising human nucleated red blood cell precursors, red blood cells, or enucleated precursors thereof, or any combination thereof, wherein the preparation comprises EPO or an EPO analog at less than 1, 0.5, 0.3 or 0.1 units/ml, or lacks EPO or an EPO analog.

In some embodiments, one or more of the nucleated red blood cell precursors comprises a vector, e.g., a vector encoding a protein of interest. In some embodiments, one or more of the red blood cells or enucleated precursors thereof comprises an exogenous protein

In some embodiments, the preparation further comprises insulin (e.g., from 5 μg/ml to 20 μg/ml insulin), holo transferrin (e.g., from 400 μg/ml to 600 μg/ml holo human transferrin), or both of insulin and holo transferrin.

Some aspects of the disclosure provide fusion proteins that comprise (i) a red blood cell transmembrane protein, and (ii) a first antigen binding protein that binds to a first epitope of an antigen. In some embodiments, the antigen is a toxin or an antigen of a pathogen. In some embodiments, the red blood cell transmembrane protein is glycophorin A (GPA) or Kell. In some embodiments, the toxin is botulinum toxin (e.g., botuninum toxin types A, B, C, D, E, F or G). In some embodiments, the toxin is botulinum toxin is botulinum toxin A, B, or E. In some embodiments, the pathogen is a virus or bacterium. In some embodiments, the first antigen binding protein is a single-domain antibody. In some embodiments, the single-domain antibody is a single-domain heavy chain antibody (VHH).

In some embodiments, the fusion protein comprises (iii) a second antigen binding protein that binds to a second epitope of the antigen. In some embodiments, the second antigen binding protein is a single-domain antibody. In some embodiments, the single-domain antibody is a single-domain heavy chain antibody (VHH). In some embodiments, the red blood cell transmembrane protein of (i) and the first antigen binding protein of (ii) are fused via a linker. In some embodiments, the first antigen binding protein of (ii) and the second antigen binding protein of (iii) are fused via a linker. In some embodiments, the first antigen binding protein of (ii) is fused N-terminal to the red blood cell transmembrane protein of (i). In some embodiments, the first antigen binding protein of (ii) is fused C-terminal to the red blood cell transmembrane protein of (i).

In some embodiments, the fusion protein comprises the structure:

### N′-[first VHH]-[glycophorin A]-C′;

### N′-[second VHH]-[first VHH]-[glycophorin A]-C′;

### N′-[Kell]-[first VHH]-C′; or

N′-[Kell]-[first VHH]-[second VHH]-C′, wherein “]-[” indicates an optional linker. In some embodiments, the first VHH and the second VHH bind different epitopes of the antigen. In some embodiments, the fusion protein comprises the amino acid sequence of (SEQ ID NO: 2), (SEQ ID NO: 4), (SEQ ID NO: 6) or (SEQ ID NO: 8).

Some aspects of the disclosure provide genetically engineered CD34⁺ progenitor cells (and cells, e.g., blood cells, descended therefrom) that express any of the fusion proteins provided herein. Some aspects of the disclosure provide genetically engineered enucleated blood cells that express any of the fusion proteins provided herein.

Some aspects of the disclosure provide nucleic acids that encode any of the fusion proteins provided herein. In some embodiments, any of the nucleic acids encoding any of the fusion proteins provided herein are comprised in a vector. In some embodiments, the nucleic acid is in operable linkage to a promoter. In some embodiments, the vector is a lentivirus vector or a retrovirus vector.

Some aspects of the disclosure provide methods of neutralizing a toxin or a pathogen in a subject, the method comprising administering to a subject in need thereof a first dose of any of the genetically engineered enucleated blood cells provided herein. In some embodiments, the toxin is botulinum toxin (e.g., botuninum toxin types A, B, C, D, E, F or G). In some embodiments, the toxin is botulinum toxin is botulinum toxin A, B, or E. In some embodiments, the pathogen is a virus or bacterium. In some embodiments, the enucleated blood cell is autologous. In some embodiments, the enucleated blood cell is allogenic. In some embodiments, the subject is infected with Clostridium botulinum. In some embodiments, the subject has ingested, inhaled, been injected with, or otherwise been exposed to botulinum toxin (e.g., botuninum toxin types A, B, C, D, E, F or G). In some embodiments the subject has ingested, inhaled, been injected with, or otherwise been exposed to botulinum toxin within 0-7 days prior to administration of the enucleated red blood cell, e.g., the subject was exposed within 24 hours or less, within 48 hours or less, within 72 hours or less, prior to administration of the enucleated red blood cell. In some embodiments the subject exhibits one or more symptoms of botulism. In some embodiments, the subject is administered a second dose of the genetically engineered enucleated blood cell at any time following the administration of the first dose of the enucleated blood cell. For example, the subject is administered a second dose of the genetically engineered enucleated blood cell 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 days, or more following the administration of the first dose of the enucleated blood cell. In some embodiments, the subject is administered a second dose of the genetically engineered enucleated blood cell from 1 day to 5 days, from 1 day to 10 days, from 1 day to 15 days, from 1 day to 20 days, from 1 day to 25 days, from 1 day to 30 days, from 1 day to 40 days, from 1 day to 50 days, from 5 days to 10 days, from 5 days to 15 days, from 5 days to 20 days, from 5 days to 25 days, from 5 days to 30 days, from 5 days to 40 days, from 5 days to 50 days, from 10 days to 15 days, from 10 days to 20 days, from 10 days to 25 days, from 10 days to 30 days, from 10 days to 40 days, from 10 days to 50 days, from 15 days to 20 days, from 15 days to 25 days, from 15 days to 30 days, from 15 days to 40 days, from 15 days to 50 days, from 20 days to 25 days, from 20 days to 30 days, from 20 days to 40 days, from 20 days to 50 days, from 25 days to 30 days, from 25 days to 40 days, from 25 days to 50 days, from 30 days to 40 days, from 30 days to 50 days, or from 40 days to 50 days following the administration of the first dose of the enucleated blood cell. In some embodiments, the subject is administered an additional dose (e.g., third, fourth, fifth, sixth, seventh, eighth, ninth, tenth, or more) of the genetically engineered enucleated blood cell following the administration of the first dose of the enucleated blood cell. In some embodiments, the enucleated blood cell is produced using any of the cell culture methods provided herein.

Some aspects of the disclosure provide a genetically engineered enucleated blood cell, the method comprising transfecting a CD34⁺ progenitor cell with a vector encoding a protein of interest and culturing the CD34⁺ progenitor cell comprising the vector according to the methods provided herein. In some embodiments, the CD34⁺ progenitor cell is transfected with any of the vectors provided herein.

Some aspects of the disclosure provide methods of producing a genetically engineered enucleated blood cell, the method comprising transfecting a CD34⁺ progenitor cell with a vector encoding a protein of interest, and administering the CD34⁺ progenitor cell comprising the vector to a subject, wherein the CD34⁺ progenitor cell differentiates into an enucleated blood cell expressing the protein of interest encoded by the vector. Some aspects of the disclosure provide methods of producing a genetically engineered enucleated blood cell, the method comprising providing a CD34⁺ progenitor cell that has been transfected with a vector encoding a protein of interest, and administering the CD34⁺ progenitor cell comprising the vector to a subject, wherein the CD34⁺ progenitor cell differentiates into an enucleated blood cell expressing the protein of interest encoded by the vector. In some embodiments, the vector is any of the vectors provided herein. In some embodiments, the subject is infected with or suspected of being infected with a pathogen. In some embodiments, the pathogen is Clostridium botulinum. In some embodiments, the subject has ingested, inhaled, been injected with, or otherwise been exposed to a toxin, e.g., a bacterial toxin, e.g., botulinum toxin, or is suspected to have ingested, inhaled, been injected with, or otherwise been exposed to a toxin, e.g., a bacterial toxin, e.g., botulinum toxin. In some embodiments, the subject is at risk of being exposed to a toxin, e.g., a bacterial toxin, e.g., botulinum toxin.

The details of certain embodiments of the invention are set forth in the Detailed Description of Certain Embodiments, as described below. Other features, objects, and advantages of the invention will be apparent from the Definitions, Examples, Figures, and Claims.

### BRIEF DESCRIPTION OF THE DRAWINGS

The accompanying drawings, which constitute a part of this specification, illustrate several exemplary embodiments of the invention and together with the description, serve to explain the principles of the invention. The embodiments disclosed in the drawings are exemplary and do not limit the scope of this disclosure.

FIGS. 1A-1E show that genetically engineered murine RBCs covalently linked to VHHs against BoNT/A protect neurons in vitro and mice in vivo against BoNT/A challenge. (FIG. 1A) Include schematic representations of the exemplary chimeric proteins. Each chimera was assembled from different protein segments shown as colored boxes (A: signal peptide of GPA; B: myc epitope; VHH-H7 and VHH-B5: anti-BoNT/A VHHs; C—spacer; VHH-D10 and VHH-G10: anti-BoNT/B VHHs; GPA: Human glycophorin A) (FIG. 1B) Are data showing RBC potency to neutralize BoNT/A assessed by SNAP25 immunoblot following overnight treatments of primary rat neurons exposed to 20 pM BoNT/A pre-incubated with the indicated number of myc⁺ RBCs. The percent of SNAP25 cleaved by BoNT/A was estimated by image analysis. Left: RBCs expressing either GPA-VNA/A or Kell-VNA/A; Right: RBCs expressing GPA-VNA/B or Kell-VNA/B. (FIG. 1C) Shows complete blood counts of control 7 week old female C57BL/6J mice and mice subjected to bone marrow transplantation with progenitor cells expressing vector or GPA-VNA/A and bled at the indicated time points. (FIG. 1D) Shows Myc surface expression on red cells from GPA-VNA/A transplanted mice was measured by flow cytometry at the indicated time points; the percentage of myc+ cells was determined. (FIG. 1E) Shows Kaplan-Meier survival plots of mice challenged with BoNT/A. CD1 mice were subjected to bone marrow transplantation with progenitor cells expressing GPA-VNA/A or Kell-VNA/A. After bone marrow reconstitution these mice were challenged with 10 LD₅₀ BoNT/A and monitored for a week. The surviving mice received increasing doses of BoNT/A in subsequent weeks. The mice bearing Kell-VNA/A RBCs were challenged up to 3 weeks before protection faltered whereas the mice carrying GPA-VNA/A were challenged for up to 6 weeks with increasing doses as indicated and are still living. (n=5/group) All mice with RBCs expressing GPA-VNA/A survived following the final 10,000 LD₅₀ treatment without showing signs of botulism.

FIGS. 2A-2H show that mice transfused with engineered RBCs are protected against BoNT/A challenges. (FIG. 2A) C57BL/6J mice were transfused with 100 μl blood from chimeric mice previously transplanted with GPA-VNA/A—expressing progenitors. Recipient mice were challenged with 10, 100, or 1000 LD₅₀ BoNT/A 2 hr later and survival was monitored (n=6/group.) Note: the curves depicting control mice, and transfused mice challenged with 100 and 1000 LD₅₀ are overlapping. (FIG. 2B) C57BL/6J mice transfused with 400 μl blood from chimeric mice previously transplanted with GPA-VNA/A-expressing progenitors were challenged with 100 LD₅₀ BoNT/A 1 hr after transfusion and survival was monitored (n=4/group.) (FIG. 2C) Circulatory half-life of the transfused RBCs. 100 μl of blood from transplanted mice with ˜3% of their RBCs expressing vector only, GPA-VNA/A, or Kell-VNA/A, were stained with violet-trace dye and transfused into recipient mice. The fraction of transfused RBCs in recipients was analyzed by flow cytometry at the indicated time points. The violet-trace dye represents the total population of transfused red blood cells, of which only ˜3% are GFP+ and express the exogenous chimeric protein, whilst the GFP signal represents only the 3% of the transfused RBCs expressing the VNAs. (FIG. 2D) Survival plot of transfusion recipient mice treated as in the diagram were challenged with 10 LD₅₀ BoNT/A at 1 h, 4 d, 14 d and 28 d post-transfusion and monitored for 7 days (n=6/group). (FIG. 2E) Detection of serum persistence of unbound ciBoNT/A in the circulation. 2 ng ciBoNT/A was incubated with 200 μl violet stained RBCs expressing the control vector or GPA-VNA/A before they were transfused into recipient mice. The sera was collected at intervals and the amount of unbound ciBoNT/A in the serum was measured by ELISA (n=3/group.) (FIGS. 2F-2G) Detection of RBC-bound ciBoNT/A and transfused RBCs in the blood of recipient mice. 200 μl blood from wild type mice and from mice transplanted with GPA-VNA/A expressing RBCs, was stained with violet trace dye and incubated with 1 μg ciBoNT/A before transfusion into mice. Recipients were bled at the indicated time points. RBCs were subjected to flow cytometry analyses to quantify the violet-trace (total transfused RBCs, FIG. 2F), GFP (virus transduced cells, FIG. 2F), and S-tag (indirectly detecting RBC-bound ciBoNT/A, FIG. 2G). (n=3/group). In FIG. 2G, the GPA-VNA/A RBCs measures total RBCs from GPA-VNA/A chimera mice, which are a combination of non-transduced (˜97%) and transduced cells (˜3%.) To further distinguish the transduced cells, the GFP+ (GPA-VNA/A) RBCs and GFP− populations (control RBCs) were gated. The difference between the GFP+ and GFP− curves is statistically significant by ANOVA two-tailed analysis. (FIG. 2H) As detailed in Methods mice received three injections of control blood, GPA-VNA/A blood, or VNA/A protein and relative the abundance of antibody against-VNA/A in serum from these mice was examined by ELISA. Sera were diluted at indicated ratios (n=5).

FIGS. 3A-3C show RBCs expressing heterodimers of neutralizing VHHs are more protective than those expressing monomers. (FIG. 3A) Bispecific or monospecific antitoxin proteins were engineered for expression on RBCs as GPA fusions. Chimeras were engineered to include the different protein segments as shown (A—signal peptide of human glycophorin A; B—myc epitope; C—spacer). (FIG. 3B) RBC potency to neutralize BoNT/A assessed by SNAP25 immunoblot following overnight treatments of primary rat neurons exposed to 20 pM BoNT/A pre-incubated with the indicated number of myc⁺ RBCs. The percent of SNAP25 cleaved by BoNT/A was estimated by image analysis and shown below the immunoblots. (FIG. 3C) Survival plot of transfusion recipient mice challenged with BoNT/A. C57BL/6J mice were transfused with 100 μl blood from chimeric mice with blood containing 3.5% RBCs expressing either GPA-VNA/A or GPA-VHH7. Mice were then challenged with 25, 50, 100, or 200 LD₅₀ BoNT/A and monitored for 7 days. (n=5/group).

FIGS. 4A-4D show characterization of in vitro differentiated human RBCs. (FIG. 4A) Table lists the composition of the culture medium at each stage. Lower panel shows the proliferation curve of human RBCs. (FIG. 4B) Differentiating cells were characterized by flow cytometry for c-kit, CD71, CD235A and Hoechst at the end of each culture stage. (FIG. 4C) Giemsa and hemoglobin staining of human RBCs at the end of each culture stage. (FIG. 4D) In vitro-differentiated human RBCs circulate for up to 7 days in macrophage-depleted NOD/SCID mice. 500 million six stages cultured RBCs were labeled with CFSE and injected intravenously into NOD/SCID mice that have been treated with clodronate liposomes. The recipient mice were then bled at indicated time points as indicated for further flow cytometry analyses. The transfused human RBCs were identified by tracing CFSE and CD235A expression.

FIGS. 5A-5E show genetically engineered human RBCs made in culture and expressing GPA-VNA/A or Kell-VNA/A differentiate normally and protect neurons against BoNT/A challenge both in neuronal culture and in vivo. (FIG. 5A) Mobilized human CD34⁺ cells infected with lentiviruses expressing the chimeric GPA-VNA/A protein were cultured by the method detailed in FIGS. 4A-4D and expression of multiple surface proteins was examined by flow cytometery at the indicated time points. (FIG. 5B) Upper panel shows CD235A and Hoechst staining of human cells expressing GPA-VNA/A generated from CD34⁺ cells that have been cultured in vitro for 20 and 23 days. Lower panel shows Giemsa and hemoglobin staining of hRBCs expressing GPA-VNA/A at d20 and d23. (FIG. 5C) Proliferation curve during culture of mobilized human CD34⁺ cells expressing vector or GPA-VNA/A. (FIG. 5D) Human RBCs expressing GPA-VNA/A or Kell-VNA/A protect cultured neuronal cells from BoNT/A protease activity. Rat neurons were co-incubated with BoNT/A and engineered hRBCs as indicated and neuronal lysates were analyzed by Western blotting as in FIGS. 1A-1E. (FIG. 5E) Survival curve of mice challenged with BoNT/A. Human RBCs generated by in vitro culture were submitted to flow cytometry to detect the percentage of GFP+ cells before injecting into mice. 150 million GFP+ human RBCs expressing empty vector or GPA-VNA/A were transfused into NOD/SCID mice. After 30 minutes, the mice were challenged with 10 LD₅₀ BoNT/A and were observed for 7 days (n=4/group). One mouse was challenged with 10 LD₅₀ BoNT/A one day after injection of 120 million GFP+ human RBCs 1 day and was observed for 7 days.

FIGS. 6A-6G show terminally differentiated mouse red blood cells express chimeric GPA or Kell on their surface. (FIG. 6A) Indicates the level of myc surface expression of cells expressing myc-tagged chimeric surface proteins Kell-VNA/B, Kell-VNA/A, GPA-VNA/B, and GPA-VNA/A. (FIG. 6B) The copy numbers of each fusion protein were estimated. (FIG. 6C-F) Using an in vitro mouse fetal liver cell culture system it is shown that VNA-expressing cells undergo enucleation, have similar CD71 and Ter119 expression, proliferate similarly to, and have similar morphology to control cells. (FIG. 6G) Approximately 3,100,000 VNA/A proteins per red blood cell were found to be expressed.

FIG. 7 shows the protective capacity in mice that were transplanted with stem cells/progenitors expressing GPA-VNA/B. Mice were challenged with 100 LD50 BoNT/B and surviving mice were challenged with 1000 LD50 the following week. After one week, a 5000LD50 BoNT/B were administrated to surviving mice. Mice were monitored for 7 days following each challenge (n=5/group).

FIGS. 8A-8B shows detection of RBC-bound ciBoNT/A and transfused RBCs in the blood of recipient mice and show that GPA-VNA/A red blood cells bound approximately 100 times more ciBoNT/A than control red blood cells when transfused in mice that were administered ciBoNT/A. (FIG. 8A) Fraction of GPA-VNA/A expressing cells. (FIG. 8B) Fraction of cells that survive.

FIGS. 9A-9C show red blood cells that express fusion proteins (e.g., hGPA-VNA/A and hKell-VNA/A) and their ability to inhibit BoNT/A activity. (FIG. 9A) Enucleated cells express approximately 500,000 copies of hGPA-VNA/A or 120,000 copies of hKell-VNA/A as indicated by myc expression. (FIG. 9B) Control red blood cells or red blood cells expressing hGPA-VNA/A that were generated using a six-stage culture method and found o contain similar amounts of GPA, Kell, β1-integrin, band 3, and XK proteins. (FIG. 9C) The observed difference in protection response is not due to different cell numbers remaining th the circulation of NOD/SCID mice, as the percentage of cells expressing control vectors or GPA-VNA/A surviving in NOD/SCID mice is similar from 5 minutes-post-transfection to 1 hour.

### DETAILED DESCRIPTION OF CERTAIN EMBODIMENTS

Red blood cells are the most numerous cell type in blood and account for a quarter of the total number of cells in the human body. RBCs possesses many unique characteristics that make them an attractive tool in therapeutics and diagnostics for various purposes, e.g., for in vivo delivery of natural or synthetic payloads. Yoo et al., 2011, Nature Reviews. Drug Delivery 10(7):521-535. For example, mature red blood cells do not have nuclei (i.e., enucleated) and thus there will be no risk of delivering remnants of foreign genes into a host. Thus, the possibility of tumorigenicity, a key risk of stem cell-based therapies (Gruen et al., 2006 Stem cells 24(10):2162-2169), is thereby eliminated. Further, red blood cells have a long lifespan in vivo, e.g., about 120 days in the human blood stream, and about 50 days in mice, and presence throughout the macro- and micro-circulation. Modification of red cells with bioavailable therapeutics might thus lead to prolonged efficacy and coverage of all areas perfused by the circulation in vivo. Moreover, RBCs have large cell surface areas of about 140 μm² with a favorable surface to volume ratio. Also, red blood cells have good biocompability when used as carriers for delivery of therapeutic or diagnostic agents. Finally, old or damaged RBCs can be removed by cells of the reticuloendothelial system. Thus, any modification made to the DNA of RBC precursors is eliminated upon their enucleation and cannot lead to abnormal growth or tumorigenicity after their transfusion into a recipient.

Accordingly, it is of great interest to develop methods for producing enucleated red blood cells, and in particular red blood cells that carry an agent of interest, such as diagnostic or therapeutic agents. Such enucleated red blood cells can be used for, e.g., delivering the agent of interest into a subject.

Engineered RBCs have been generated using encapsulation (Biagiotti et al., 2011, IUBMB life 63(8):621-631; Godfrin et al., 2012, Expert Opinion on Biological Therapy 12(1):127-133; and Muzykantov, 2010, Expert Opinion on Drug Delivery 7(4):403-427), by non-covalent attachment of foreign peptides, or through installation of proteins by fusion to a monoclonal antibody specific for a RBC surface protein (Murciano, 2003, Nature Biotechnology 21(8):891-896; and Zaitsev et al., 2010, Blood 115(25):5241-5248). Modified RBCs face limitations if intended for application in vivo. Encapsulation allows entrapment of sizable quantities of material, but at the expense of disrupting plasma membrane integrity, with a concomitant reduction in circulatory half-life of the modified red blood cells. Osmosis driven entrapment limits the chemical nature of materials that can be successfully encapsulated, the site of release is difficult to control, and encapsulated enzymes are functional only at the final destination, compromising reusability at other sites. Murciano et al., 2003 and Zaitsev et al., 2010. Targeting of cargo to RBCs by fusion to an RBC-specific antibody, (e.g., antiglycophorin antibody), has its limitations because this mode of attachment to the RBC is non-covalent and readily dissociates, thus reducing circulatory half life and mass of cargo available for delivery. Murciano et al., 2003 and Zaitsev et al., 2010. Other developments that exploit RBCs for targeted delivery include nanoparticles enveloped by an RBC-mimicking membrane as well as RBC-shaped polymers. Yoo et al., 2011 Nature Reviews. Drug Discovery 10(7):521-535. The short in vivo survival rate of these RBC-inspired carriers (˜7 days maximum) may limit their therapeutic utility.

Suitable cell culture methods for generating mature red blood cells have been disclosed. See Lee H Y, et al. PPAR-alpha and glucocorticoid receptor synergize to promote erythroid progenitor self-renewal. Nature 522, 474-477 (2015); and Griffiths R E, et al. Maturing reticulocytes internalize plasma membrane in glycophorin A-containing vesicles that fuse with autophagosomes before exocytosis. Blood 119, 6296-6306 (2012). However, with respect to the existing technology, there is a need to develop new methodology for culturing and engineering RBCs, such that they can be produced more quickly, more efficiently, and carry a wide variety of useful cargoes to specific locations in the body. To this point, the cell culture methods provided herein yielded a 3 fold improvement in the extent of enucleation (more than 90% enucleation) and a 2 fold increase in cell yield as compared to previously described culturing processes.

In some aspects, technology described herein relates to the use of genetic methods to modify progenitors of red blood cells such that the red blood cells that are formed express on their surface fusion proteins of glycophorin A or Kell with proteins of interest. Proteins of interest include, without limitation, camelid heavy chain only antibodies (termed VHHs) (FIG. 10), which may be used for neutralizing toxins and/or neutralizing foreign pathogens.

Antigen binding proteins that were tested include two heterodimers of VHHs (VNA) targeting different epitopes of botulinum toxin A (BoNT/A) or B. Constructs of these fusion genes both were made in the retrovirus transduction vector (XZ-201) and lentivirus transduction vectors (HMD.) These constructs were expressed in 293T cells to generate transducing viruses and the virus was used for transducing mouse erythrocyte progenitors and human CD34 cells. 1 ml of retrovirus was used for transducing 100,000 erythrocyte progenitors and 1 ml of lentivirus was used for transducing 125,000 CD34 cells at the end of stage 1 during CD34 differentiation.

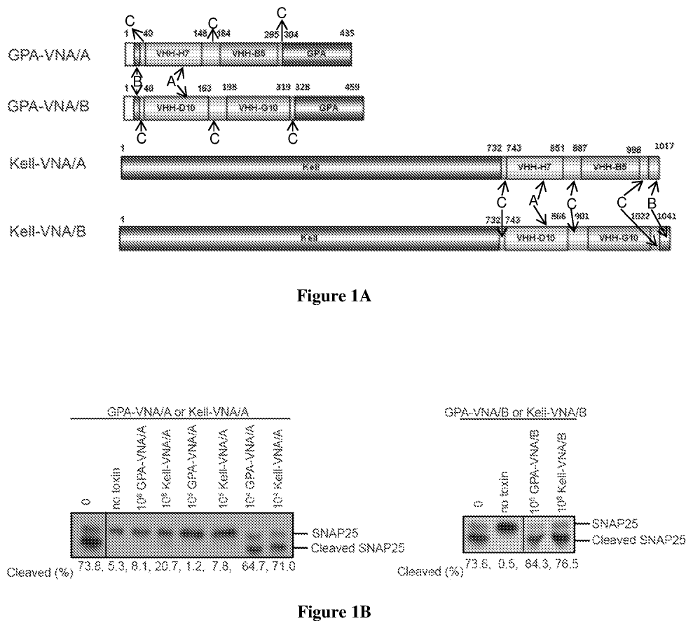

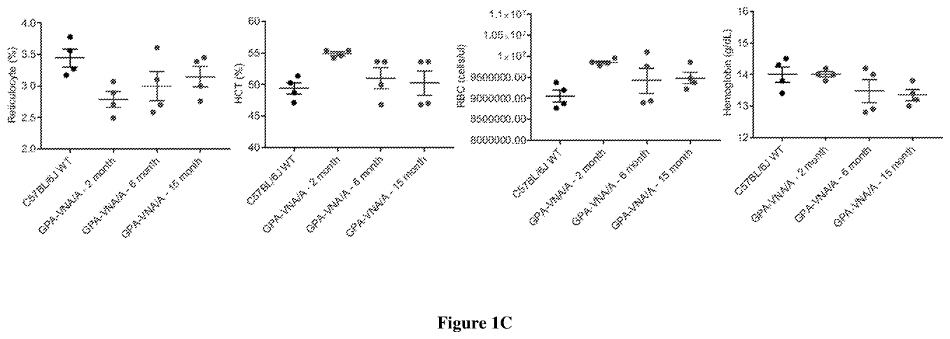

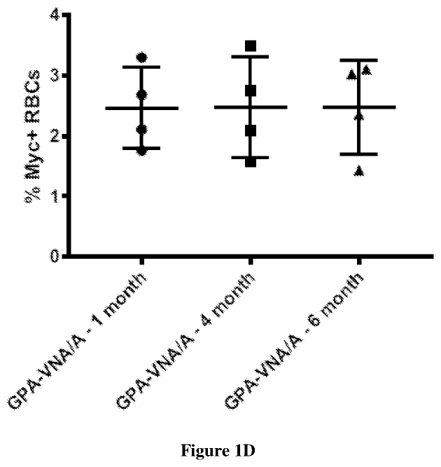

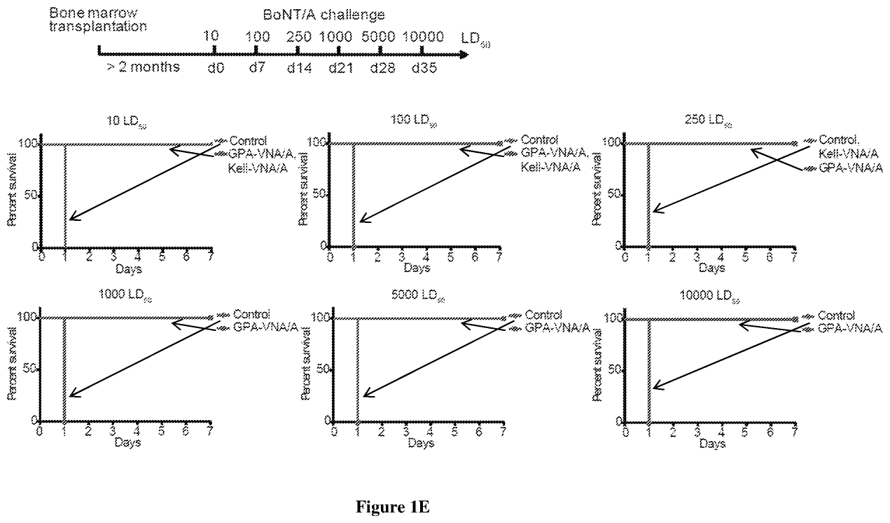

It was shown that these fusion proteins are expressed on the red cell surface at the end of differentiation as indicated by the expression of the Myc tag which was included in the fusion proteins (FIG. 1A). Importantly, transduction and culture methods did not affect red cell differentiation as normal enucleation rates and normal Giemsal benzidine staining was observed at the end of the culture.

It was shown that 100,000 human red blood cells expressing either GPA or Kell-VNA anti-BoNT/A, produced in a 6-step in vitro cultured system, can neutralize 20 pM BoNT/A as determined by preventing BoNT/A mediated-cleavage of the SNAP-25 protein in cultured neuronal cells after incubating red blood cells, BoNT/A and primary neurons overnight (FIG. 1B, left). Control red blood cells expressing VHHs targeting Botulinum toxin B had no significant effect (FIG. 1B, right).

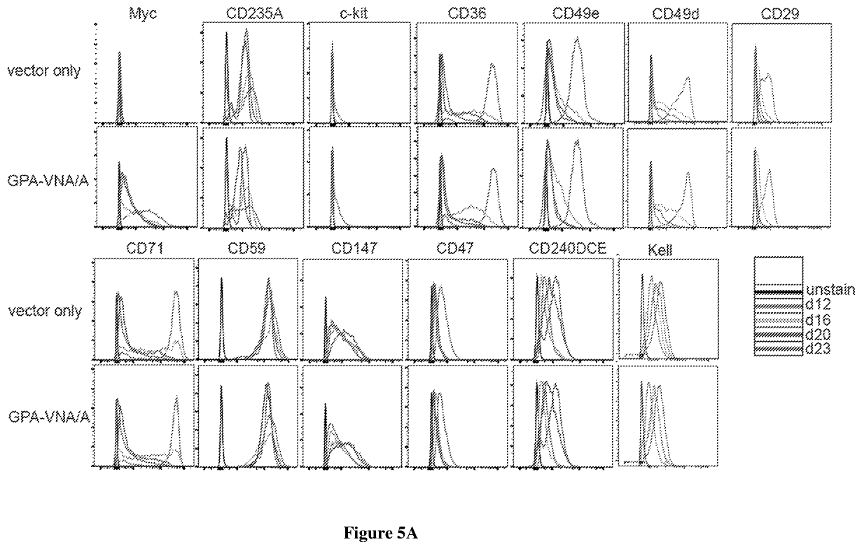

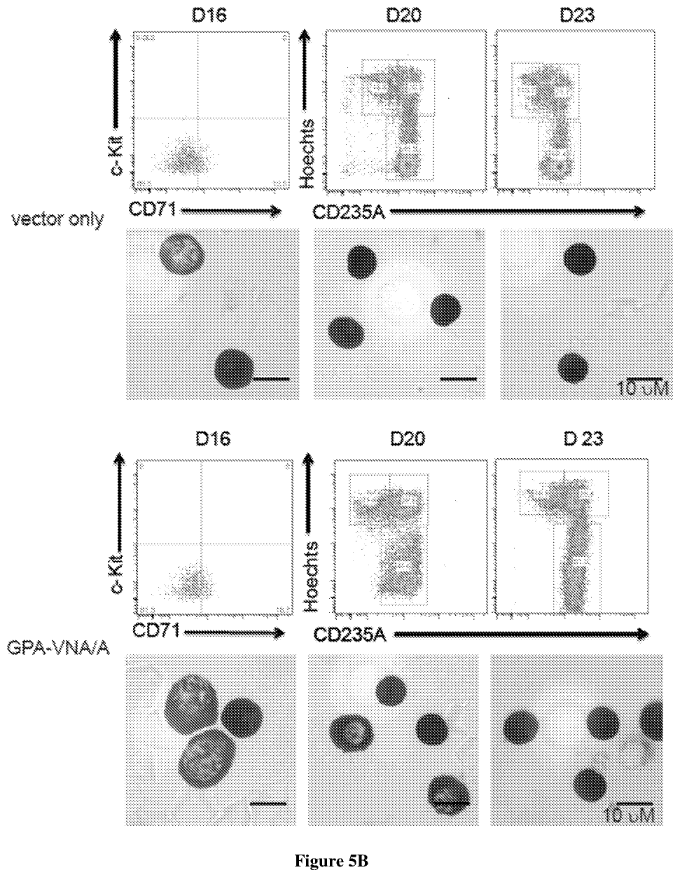

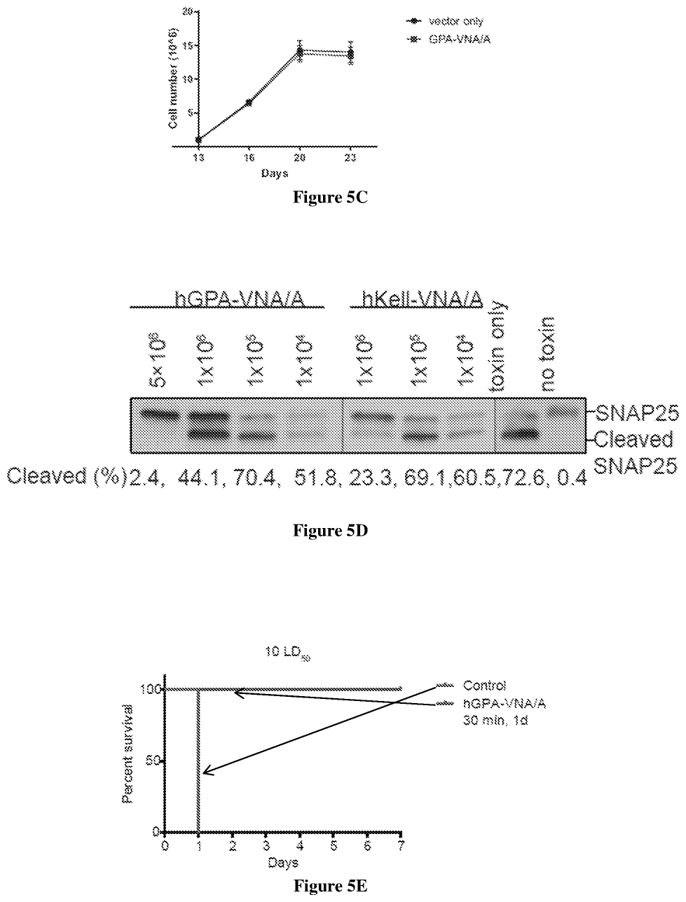

It was also shown that 5 million human red blood cells expressing GPA-VNA anti-BoNT/A or 1 million human red blood cells expressing Kell-VNA anti-BoNT/A can neutralize 20 pM BoNT/A in the same setting as employed with mouse red blood cells (FIG. 5D).

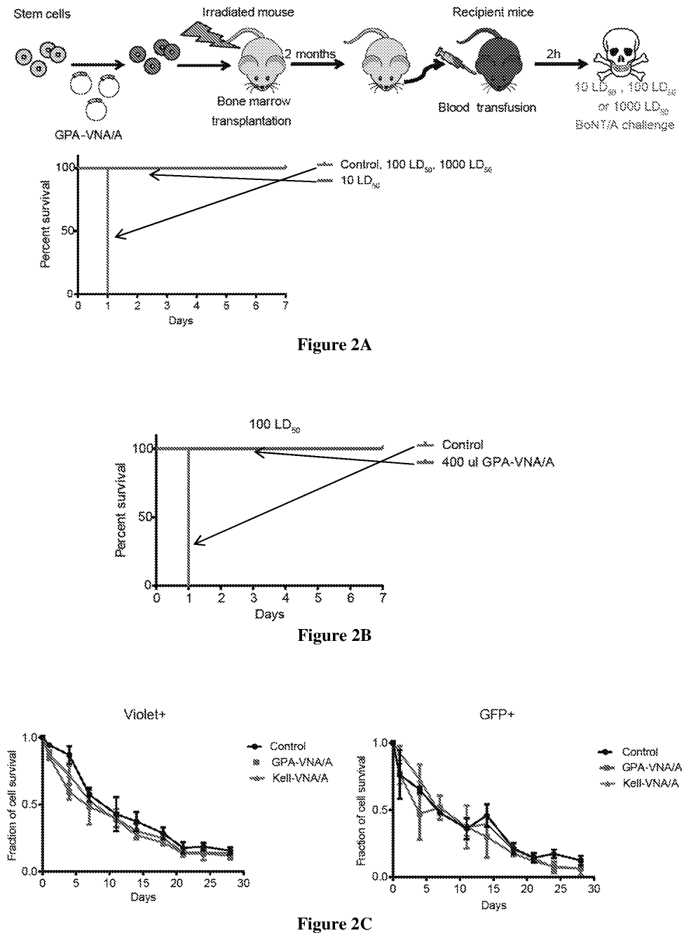

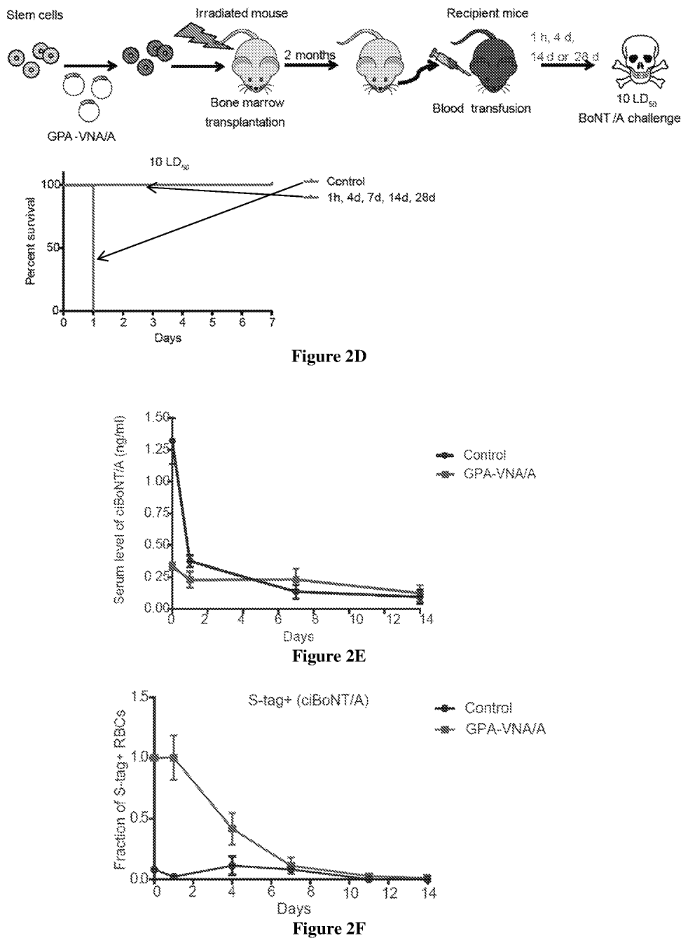

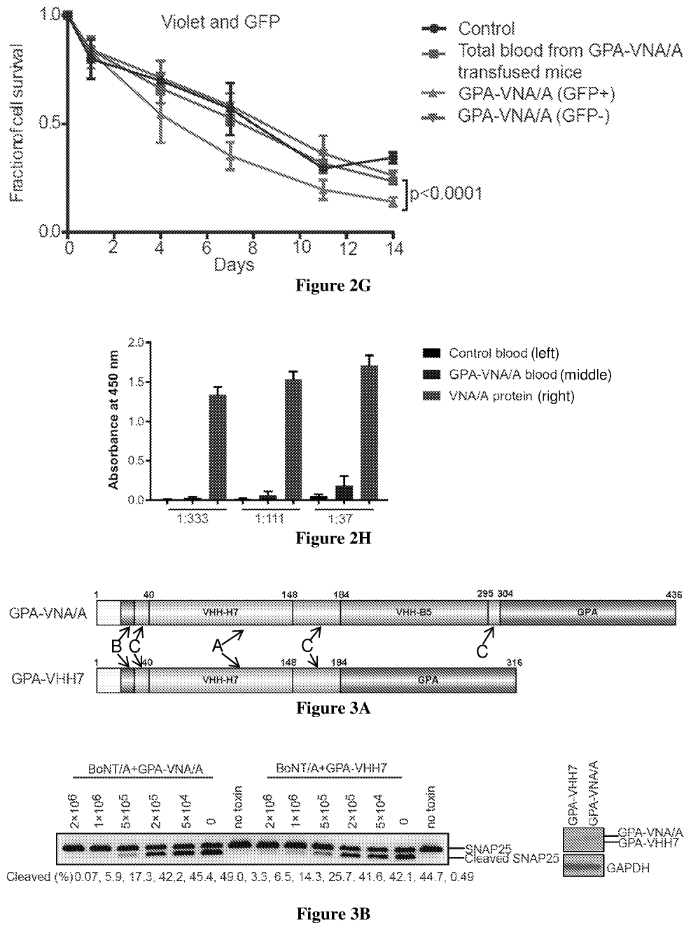

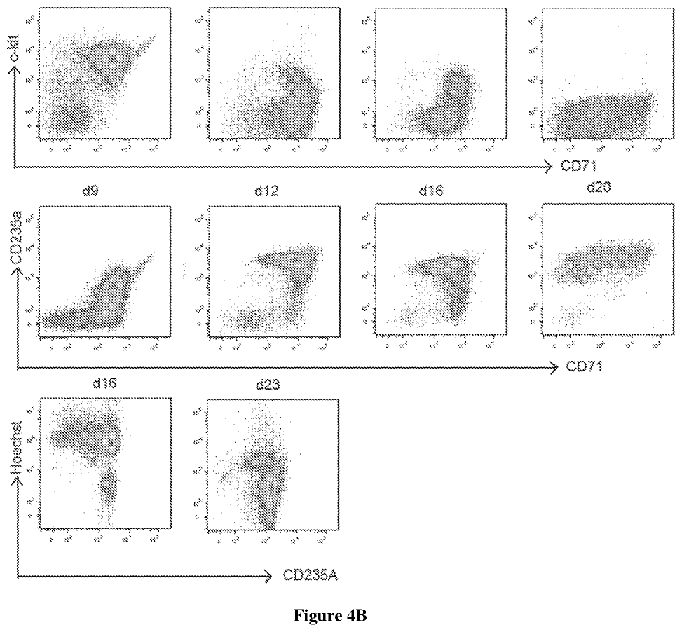

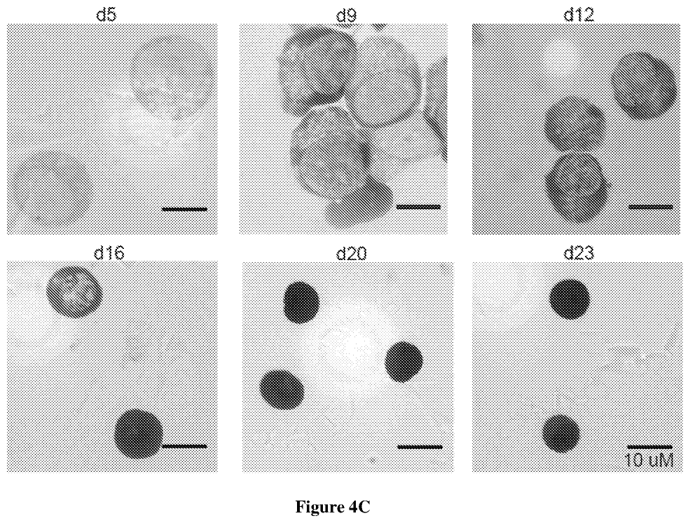

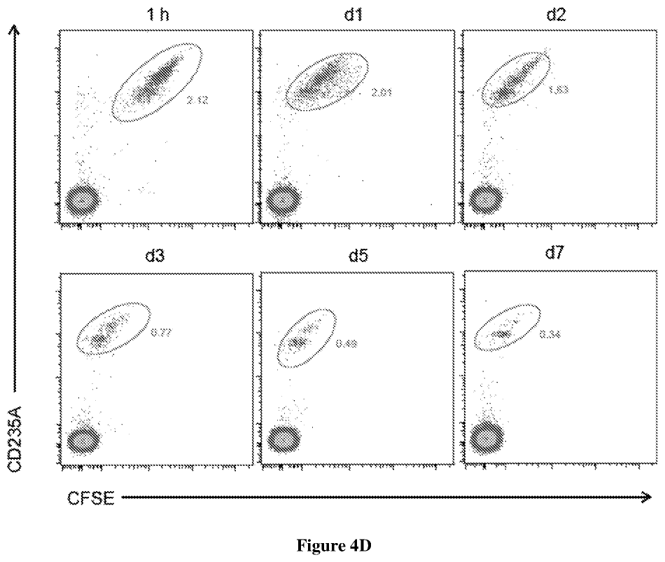

In addition to in vitro toxin neutralization, it was also shown that VHH engineered red blood cells are effective for botulism protection in vivo, both in mice transplanted with engineered red blood cell producing progenitors (FIG. 1E) and in mice receiving blood from the transplanted mice employed in FIG. 1E (FIGS. 2A, 2B, and 2D). This protection lasts up to 28 days post transfusion. This protection ability of engineered red cells was also shown in NOD/SCID mice transfused with human red blood cells expressing GPA-VNA-anti-BoNT/A made in the CD34⁺ cell culture system described herein (FIG. 5E). Of note, the CD34⁺ cell culture system used is an improvement over previous methods and significantly improves the production of sufficient human engineered red blood cells used in these assays. In certain cases, the culture system described herein can provide a 3 fold improved extent of enucleation (more than 90% enucleation) and/or a 2 fold increased cell yield compared to the system previously described (FIG. 4C). See Lee H Y, et al. PPAR-alpha and glucocorticoid receptor synergize to promote erythroid progenitor self-renewal. Nature 522, 474-477 (2015); and Griffiths R E, et al. Maturing reticulocytes internalize plasma membrane in glycophorin A-containing vesicles that fuse with autophagosomes before exocytosis. Blood 119, 6296-6306 (2012).

The in vitro generation of human RBCs and genetic engineering of their precursors as described herein may provide a robust platform for application of this surface engineering method, e.g., in conjunction with cytosolic modification, to clinical applications (Liu et al., 2010 Blood 115(10):2021-2027; Cong et al., 2013 Science 339(6121):819-826; Mali et al., Science 339(6121):823-826; Giarratana et al., 2011 Blood 118(19):5071-5079; Douay et al., 2009 Blood 105(1):85-94; and Griffiths et al., 2012 Blood 119(26):6296-6306.) Moreover, the established safety of blood transfusions inspires confidence that these engineered red blood cells will indeed find use in humans.

Accordingly, described herein is an improved in vitro multi-phase culturing process for producing enucleated red blood cells from mobilized CD34⁺ progenitor cells (e.g., human mobilized CD34⁺ peripheral blood cells). Such enucleated red blood cells can be genetically engineered such that they express proteins of interest, e.g., fusion proteins comprising a red blood cell transmembrane protein fused to one or more VHH domains (e.g., botulinum toxin-binding VHH domain(s)). Also described herein are methods of neutralizing a toxin or a pathogen in a subject by administering to a subject in need thereof a dose of any of the genetically engineered enucleated blood cells provided herein.

### Culturing Systems for Producing Enucleated Red Blood Cells:

Described herein is an in vitro culturing process for producing mature enucleated red blood cells from CD34⁺ progenitor cells (e.g., from a human subject). This culturing process involves multiple differentiation stages (e.g., 2, 3, 4, 5, 6 or more) and optionally an expansion stage prior to the differentiation stages. In some embodiments, the total time period for the in vitro culturing process described herein can range from 9-33 days (e.g., 15-33 days, 15-29 days, or 18-25 days). In one example, the total time period is 23 days. In some embodiments, one or more of the differentiation stages comprises culturing cells in a medium comprising erythropoietin (EPO) and/or stem cell factor (SCF). In some embodiments, an amount of EPO used in the cell culture medium of a differentiation stage (e.g., a first, second, third, fourth, or fifth differentiation stage) is decreased in the cell culture medium of one or more subsequent differentiation stages. In some embodiments, an amount of SCF used in the cell culture medium of a differentiation stage (e.g., a first, second, third, fourth, or fifth differentiation stage) is decreased in the cell culture medium of one or more subsequent differentiation stages. In some embodiments, one or more of the differentiation stages comprises culturing cells in medium comprising transferrin. In some embodiments, an amount of transferrin used in the cell culture medium of a differentiation stage (e.g., a first, second, third, fourth, or fifth differentiation stage) is increased in the cell culture medium of one or more subsequent differentiation stages. In some embodiments the cell culture process provided herein includes 2, 3, 4, 5, 6, 7, 8, 9, or 10 differentiation stages. As one example, the cell culture process includes 5 differentiation stages. It should be appreciated that any of the cell culture systems provided herein may include one or more expansion stages, which are performed prior to one or more differentiation stages.

### CD34⁺ Progenitor Cells

CD34 is a cell surface glycoprotein and functions as a cell-cell adhesion factor. Many human progenitor cells express this cell surface marker. Novershtern et al., Cell 144:296-309, 2011. A progenitor cell, like a stem cell, has a tendency to differentiate into a specific type of cell. Progenitor cells are usually more specific than stem cells and are often pushed to differentiate into the target cells. Any type of CD34⁺ progenitor cells that possess the tendency of differentiating into red blood cells can be used in the in vitro culturing process described herein. Such progenitor cells are well known in the art. See, e.g., Novershtern et al., Cell 144:296-309, 2011. In some examples, the in vitro culturing process described herein utilizes mobilized CD34⁺ peripheral blood cells as the progenitor cells for differentiation into enucleated red blood cells. CD34⁺ progenitor cells can also be derived from other sources (e.g., bone marrow).

Various techniques can be used to separate or isolate the CD34⁺ cell population from a suitable source such as peripheral blood cells. For example, antibodies such as monoclonal antibodies binding to CD34 can be used to enrich or isolate CD34⁺ cells. The anti-CD34 antibodies can be attached to a solid support such that cells expressing these surface markers are immobilized, thereby allowing for the separation of CD34⁺ cells from cells that do not express this surface marker. The separation techniques used should maximize the retention of viable cells to be collected. Such separation techniques can result in sub-populations of cells where up to 10%, usually not more than about 5%, preferably not more than about 1%, of the selected cells do not express CD34. The particular technique employed will depend upon the efficiency of separation, associated cytotoxicity, ease and speed of performance, and necessity for sophisticated equipment and/or technical skill.

An “isolated” or “purified” population of CD34⁺ cells for use in the in vitro culturing process described herein is substantially free of cells and materials with which it is associated in nature, in particular, free of cells that lack the desired phenotype, e.g., expressing CD34. Substantially free or substantially purified includes at least 50% CD34⁺ cells, preferably at least 70%, more preferably at least 80%, and even more preferably at least 90% CD34⁺ cells.

Procedures for separating the CD34⁺ population of cells can include, but are not limited to, physical separation, magnetic separation, antibody-coated magnetic beads, affinity chromatography, cytotoxic agents joined to a monoclonal antibody or used in conjunction with a monoclonal antibody, including, but not limited to, complement and cytotoxins, and “panning” with antibody attached to a solid matrix, e.g., plate, elutriation or any other convenient technique.

The use of physical separation techniques also include those based on differences in physical (density gradient centrifugation and counter-flow centrifugal elutriation), cell surface (lectin and antibody affinity), and vital staining properties (mitochondria-binding dye rho123 and DNA-binding dye Hoechst 33342). These procedures are well known to those of skill in this art.

Techniques providing accurate separation of CD34⁺ cells further include flow cytometry, which can have varying degrees of sophistication, e.g., a plurality of color channels, low angle and obtuse light scattering detecting channels, impedance channels. CD34⁺ cells also can be selected by flow cytometry based on light scatter characteristics, where the target cells are selected based on low side scatter and low to medium forward scatter profiles.

### Expansion Stage

Optionally, the in vitro culturing process described herein includes an expansion stage, in which the CD34⁺ progenitor cells are allowed to proliferate. As used herein, expansion or proliferation includes any increase in cell number. For example the expansion may include a 2 fold, 10 fold, 50 fold, 100 fold, 500 fold, 1000 fold, or 10000 fold increase in cell number. Expansion includes, for example, an increase in the number of CD34⁺ cells over the number of CD34⁺ cells present in the cell population used to initiate the culture. Expansion can also include increased survival of existing CD34⁺ cells. The term survival refers to the ability of a cell to continue to remain alive or function.

A population of CD34⁺ progenitor cells (e.g., mobilized CD34⁺ peripheral blood cells) can be placed in a suitable container for expanding the CD34⁺ cells. For example, suitable containers for culturing the population of cells include flasks, tubes, or plates. In one embodiment, the flask can be T-flask such as a 12.5 cm², or a 75 cm² T-flask. The plate can be a 10 cm plate, a 3.5 cm plate, or a multi-welled plate such as a 12, 24, or 96 well plate. The wells can be flat, v-bottom, or u-bottom wells. The containers can be treated with any suitable treatment for tissue culture to promote cell adhesion or to inhibit cell adhesion to the surface of the container. Such containers are commercially available from Falcon, Corning and Costar. As used herein, “expansion container” also is intended to include any chamber or container for expanding cells whether or not free standing or incorporated into an expansion apparatus.

The starting cell density of the cultured population of CD34⁺ cells can be from about 1×10² cells to about 1×10⁷ cells/mL. In some embodiments, the density of the cultured population of CD34⁺ cells is from 1×10² to 1×10⁷ cells/mL. In some embodiments, the density of the cultured population of CD34⁺ cells is from 1×10² to 1×10³ cells/mL, from 1×10² to 1×10⁴ cells/mL, from 1×10² to 1×10⁵ cells/mL, from 1×10² to 1×10⁶ cells/mL, from 1×10³ to 1×10⁴ cells/mL, from 1×10³ to 1×10⁵ cells/mL, from 1×10³ to 1×10⁶ cells/mL, from 1×10³ to 1×10⁷ cells/mL, from 1×10⁴ to 1×10⁵ cells/mL, from 1×10⁴ to 1×10⁶ cells/mL, from 1×10⁴ to 1×10⁷ cells/mL, from 1×10⁵ to 1×10⁶ cells/mL, from 1×10⁵ to 1×10⁷ cells/mL, or from 1×10⁶ to 1×10⁷ cells/mL. Preferably, the cell density can be from about 1×10⁵ to about 1×10⁶ cells/mL. The cells can be cultured at an oxygen concentration of from about 2 to 20% (e.g., 2%, 3%, 4%, 5%, 6%, 7%, 8%, 9%, 10%, 11%, 12%, 13%, 14%, 15%, 16%, 17%, 18%, 19%, or 20%). In some embodiments, the cells are grown in a suspension culture.

Various cell culture media and supplements can be used to expand the population of CD34⁺ progenitor cells. As used herein, cell culture medium that is used to expand a population of CD34⁺ cells, for example in an expansion stage, is referred to herein as “expansion medium”. In some embodiments, the expansion medium comprises one or a mixture of the following base media: StemSpan™ media (e.g., Serum-Free Expansion Media, SFEM or SFEM II), Dulbecco's MEM (DMEM), IMDM, DMEM/F12, MEM, Opti-MEM, ISCOVE, HAM F12, HAM F10, M199, L15, 6M NCTC109 medium, Fischer medium, Waymouth medium, VPSFM medium, Williams medium, X-Vivo 15 (e.g., serum-depleted), RPMI (e.g., RPMI-1640), StemMACS™ HSC Expansion media, HPGM (Cambrex, Walkersville, Md.), QBSF-60 (Quality Biological, Gaithersburg, Md.), StemPro-34 (Invitrogen, Carlsbad, Calif.), X-vivo 20(BioWhittake), Stemline (Sigma) and StemSpan H3000, and StemMACS HSC Expansion Media. However, it should be appreciated that other base media suitable for expanding CD34⁺ cells may be used and the examples of base media provided herein are not meant to be limiting.

These base media used in the expansion medium, described above, as well as the base media used in one or more of the differentiation media (e.g., first, second, third, fourth, or fifth differentiation medium), described below, may be enriched according to the needs of the cells, with additional nutrient factors such as, for example, sugars such as glucose, amino acids such as glutamine, a cocktail of nonessential amino acids or of essential amino acids or of peptides, acids or acid salts such as sodium pyruvate, EDTA salts, citric acid derivatives or more generally derivatives of acids involved in the Krebs cycle, alcohols such as ethanol, amino alcohols such as ethanolamine, vitamins such as vitamin C and vitamin E, antioxidants such as glutathione or selenium, fatty acids with saturated or unsaturated chains such as linoleic acid, arachidonic acid, oleic acid, stearic acid or palmitic acid, lipids or lipopeptides, and also with phospholipids such as lecithins. The addition of a buffer solution based on HEPES or bicarbonates may prove to be necessary for certain fragile cell cultures or for cultures producing large amounts of CO₂, or optionally to buffer culture media that are highly supplemented with acids. In general, care will be taken to ensure that the pH of the culture medium remains between 6 and 8, usually between 7 and 8 and more specifically between 7.2 and 7.5. As far as is possible, care will be taken to ensure that the culture medium remains isotonic. In some embodiments, the expansion medium comprises StemSpan™ SFEM, which may be obtained from STEMCELL TECHNOLOGIES (Catalog #09600/09650). In some embodiments, the expansion medium comprises StemSpan™ SFEM II, which may be obtained from STEMCELL TECHNOLOGIES (Catalog #09605/09655). In some embodiments, the expansion medium comprises StemMACS™ HSC Expansion media, which may be obtained from Miltenyi Biotec (order #130-100-463).

In some embodiments, the expansion medium or any of the differentiation media (e.g., first, second, third, fourth, or fifth differentiation medium) is serum free and/or plasma free, meaning that the media is not supplemented with and does not contain serum and/or plasma (e.g., serum and/or plasma from a human or cow). In some embodiments one or more of the the cell culture methods provided herein are performed in the absence of serum and/or plasma. In some embodiments, the expansion medium or any of the differentiation media (e.g., first, second, third, fourth, or fifth differentiation medium) comprise one or more serum substitutes. Serum substitutes which are incorporated according to the invention may be selected from the group known in the art to be supportive of ex vivo hematopoietic stem cell (HSC) expansion, for example, serum substitutes may comprise, without limitation, Albumax, bovine serum albumin (BSA), transferrin (TF), glutamine, hydrocortisone (HC), peptone, 2-mercaptoethanol (2-ME), insulin, polyvinylpyrrolidone (PVP), Knockout Serum Replacement (KNOCKOUT™ SR, Invitrogen), Serum Replacement 1 (Sigma-Aldrich), Serum Replacement 2 (Sigma-Aldrich) and/or BIT9500 (Stemcell).

In some embodiments, the expansion medium or any of the differentiation media (e.g., first, second, third, fourth, or fifth differentiation medium) comprise serum and/or plasma (e.g., serum and/or plasma from a human or cow). Serum is commonly used as a supplement to base media in cell culture. One type of serum used for cell growth is fetal bovine serum (FBS), which is also known as fetal calf serum (FCS). In cell culture, serum provides a wide variety of macromolecular proteins, low molecular weight nutrients, carrier proteins for water-insoluble components, and other compounds that support in vitro growth of cells, such as hormones and attachment factors. Serum can also add buffering capacity to the base media and can bind or neutralize toxic components. The selection of a serum supplement for cell culture applications may depend on the chemical definition of the base medium, the type of cell to be grown, and the culture system being employed. In some embodiments, the serum and/or plasma is from a horse, pig, rabbit, goat, cow or human. In some embodiments, the expansion medium or any of the differentiation media (e.g., first, second, third, fourth, or fifth differentiation medium) comprises fetal bovine serum, newborn calf serum, or human serum.

The amount of serum and/or plasma used in the expansion medium or any of the differentiation media (e.g., first, second, third, fourth, or fifth differentiation medium) may be adjusted to support the growth and/or differentiation of one or more CD34⁺ cells. In some embodiments, the expansion medium comprises from 0.1% serum to 20% serum. In some embodiments, the expansion medium comprises from 0.1% to 0.5%, from 0.1% to 1%, from 0.1% to 2%, from 0.1% to 5%, from 0.1% to 10%, from 0.5% to 1%, from 0.5% to 2%, from 0.5% to 5%, from 0.5% to 10%, from 1% to 2%, from 1% to 5%, from 1% to 10%, from 2% to 5%, from 2% to 10%, or from 5% to 10% serum. In some embodiments, the expansion medium comprises from 0.1% serum to 20% plasma. In some embodiments, the expansion medium comprises from 0.1% to 0.5%, from 0.1% to 1%, from 0.1% to 2%, from 0.1% to 5%, from 0.1% to 10%, from 0.5% to 1%, from 0.5% to 2%, from 0.5% to 5%, from 0.5% to 10%, from 1% to 2%, from 1% to 5%, from 1% to 10%, from 2% to 5%, from 2% to 10%, or from 5% to 10% plasma.

It should be appreciated that the growth of hematopoietic tissue (e.g., CD34⁺ cells) ex vivo may be supported by the presence of several cytokines or growth factors. Several distinct factors have been identified, cloned and are now routinely manufactured as recombinant molecules for both research and/or clinical use. These include, without limitation, erythropoietin, interleukin-3 (IL-3), granulocyte macrophage-colony stimulating factor (GM-CSF), granulocyte-colony stimulating factor (G-CSF), stem cell factor (SCF), erythropoietin (EPO), and interleukin-11 (IL-11), to list a few. In some embodiments, CD34⁺ cells are cultured ex vivo in media comprising StemSpan™ CC100.

In some embodiments, the expansion medium or any of the differentiation media (e.g., first, second, third, fourth, or fifth differentiation medium) comprise one or more supplements (e.g., growth factors, hormones, and/or cytokines), which may support growth and/or differentiation of one or more CD34⁺ cells. Exemplary supplements that may be used in one or more stages of the in vitro culturing process (e.g., in the expansion stage or any of the differentiation stages described below) include, without limitation, fms like tyrosine kinase 3 (Flt-3) ligand, interleukin-2 (IL-2), interleukin 3 (IL-3), interleukin 6 (IL-6) including soluble IL-6 receptor, interleukin 12 (IL12), G-CSF, granulocyte-macrophage colony stimulating factor (GM-CSF), interleukin 1α (IL-1α), interleukin 11 (IL-11), MIP-1α, leukemia inhibitory factor (LIF), c-kit ligand. In some embodiments, the Flt-3 ligand is a naturally-occurring Flt-3 ligand, a recombinant Flt-3 ligand or a Flt-3 ligand analog which similarly affects proliferation and differentiation of blood cell progenitors. Accordingly, Flt-3 ligand may be any Flt-3 ligand or an analog of Flt-3 ligand having similar activity thereof. In some embodiments, the Flt-3 ligand is from a horse, pig, rabbit, goat, cow or human. In some embodiments, the Flt-3 ligand is from a human. In some embodiments, the Flt-3 ligand is recombinant Flt-3 ligand, for example recombinant human Flt-3 ligand.

In some examples, the in vitro culturing process described herein, or any stages thereof, can include culture conditions, in which one or more supplement is specifically excluded from the culture medium. Media supplements are commercially available from several vendors such as, for example, Amgen (Thousand Oaks, Calif.), R & D Systems and Immunex (Seattle, Wash.), Gibco, Sigma-Aldrich, VWR, and EMD Millipore. Supplements can also include fibroblast growth factor (FGF) (e.g., FGF-1 or FGF-2), insulin-like growth factor (e.g., IGF-2, or IGF-1), thrombopoietin (TPO), erythropoietin (EPO) and stem cell factor (SCF), or analogs and equivalents thereof. Equivalents thereof include molecules having similar biological activity to these factors (e.g., FGF, TPO, IGF, and SCF) in wild-type or purified form (e.g., recombinantly produced). Analogs include fragments retaining the desired activity and related molecules. For example, TPO is a ligand of the mp1 receptor, thus molecules capable of binding the mp1 receptor and initiating one or more biological actions associated with TPO binding to mp1 are also within the scope of the invention. An example of a TPO mimetic is found in Cwirla et. al. (1997) Science 276:1696. In some embodiments, the expansion medium is supplemented with StemSpan™ CC100 (100×), which contains a combination of recombinant human cytokines.

In some embodiments, the expansion medium or any of the differentiation media (e.g., first, second, third, fourth, or fifth differentiation medium) comprise one or more glucocorticoids, which are a class of corticosteroids. Exemplary corticosteroids include, without limitation, cortisol, cortisone, prednisone, prednisolone methylprednisolone dexamethasone, betamethasone, triamcinolone. In some embodiments, the expansion medium or any of the differentiation media comprises from 10 nM to 1 mM of a corticosteroid. In some embodiments, the media comprises from 10 nM to 50 nM, from 10 nM to 100 nM, from 10 nM to 200 nM, from 10 nM to 500 nM, from 10 nM to 1 mM, from 50 nM to 100 nM, from 50 nM to 200 nM, from 50 nM to 500 nM, from 50 nM to 1 mM, from 100 nM to 200 nM, from 100 nM to 500 nM, from 100 nM to 1 mM, from 200 nM to 500 nM, from 200 nM to 1 mM, or from 500 nM to 1 mM of a corticosteroid. In some embodiments, the expansion medium comprises the corticosteroid dexamethasone. In some embodiments, the expansion medium comprises from 50 nM to 150 nM of dexamethasone. In some embodiments, the expansion medium comprises about 100 nM of dexamethasone. In some embodiments, the expansion medium does not comprise a corticosteroid (e.g., dexamethasone). In some embodiments, one or more of the differentiation media comprises a corticosteroid (e.g., dexamethasone). In some embodiments, one or more of the differentiation media does not comprise a corticosteroid (e.g., dexamethasone).

In some embodiments, the expansion medium or any of the differentiation media (e.g., first, second, third, fourth, or fifth differentiation medium) are supplemented with heparin. In some embodiments, the expansion medium or any of the differentiation media comprises from 0.1 units/mL (U/mL) to 10 U/mL of heparin. In some embodiments, one unit of heparin is 0.002 mg of pure heparin, which is the quantity required to keep 1 mL of cat's blood fluid for 24 hours at 0° C. In some embodiments, the expansion medium or any of the differentiation media comprises from 0.1 U/mL to 0.5 U/mL, from 0.1 U/mL to 1 U/ml, from 0.1 U/mL to 2 U/mL, from 0.1 U/mL to 3 U/mL, from 0.1 U/mL to 5 U/mL, from 0.1 U/mL to 10 U/mL, from 0.5 U/mL to 1 U/ml, from 0.5 U/mL to 2 U/mL, from 0.5 U/mL to 3 U/mL, from 0.5 U/mL to 5 U/mL, from 0.5 U/mL to 10 U/mL, from 1 U/mL to 2 U/mL, from 1 U/mL to 3 U/mL, from 1 U/mL to 5 U/mL, from 1 U/mL to 10 U/mL, from 2 U/mL to 3 U/mL, from 2 U/mL to 5 U/mL, from 2 U/mL to 10 U/mL, from 3 U/mL to 5 U/mL, from 3 U/mL to 10 U/mL, or from 5 U/mL to 10 U/mL of heparin. In some embodiments, the expansion medium comprises heparin. In some embodiments, the expansion medium does not comprise heparin. In some embodiments, one or more of the differentiation media comprises heparin. In some embodiments, one or more of the differentiation media does not comprise heparin.

In one example, the expansion stage of the in vitro culturing process described herein can be performed as follows. A population of human mobilized CD34⁺ peripheral blood cells is placed in an expansion container at a cell density of 1×10⁴-1×10⁶ (e.g., 1×10⁵) cells/mL. In some embodiments, human mobilized CD34⁺ peripheral blood cells are mobilized with granulocyte-colony stimulating factor (G-CSF). Such cells can be purchased from the Fred Hutchinson Cancer center and thawed according to the vendor's protocol. The CD34⁺ cells are cultured in an expansion medium (e.g., StemSpan II medium) supplemented with a cytokine mixture of recombinant human Flt-3 ligand (e.g., 50 ng/mL-150 ng/mL), recombinant human SCF (e.g., 50 ng/mL-150 ng/mL), recombinant human IL-3 (e.g., 10 ng/mL-30 ng/mL), recombinant human IL-6 (e.g., 10 ng/mL-30 ng/mL), dexamethasone (e.g., 50 nM-150 nM), and 2% penicillin and streptomycin under suitable conditions (e.g., 37° C.) for 1-6 days (e.g., 2-5 days, 3-4 days, or 5 days). The expanded CD34⁺ cells can be collected and subjected to further in vitro culturing under conditions allowing for differentiation toward mature enucleated red blood cells.

In general, the above-mentioned proteins are purified or partially purified before they are added to the culture medium. Usually, they will be produced by recombinant DNA methods, but they may also be purified by standard biochemical techniques from conditioned media. Non-naturally-occurring growth factors can also be produced by recombinant DNA methods. For example, PIXY 321 is a fusion protein that has both GM-CSF and IL-3 activity. It will be evident to those skilled in the art that other fusion proteins, combining multiple growth factor activities, can be readily constructed. For example, fusion proteins combining SCF activity with that of other growth factors such as IL-1, IL-3, IL-6, G-CSF, and/or GM-CSF.

In some embodiments, the expansion medium may be substantially free of certain supplements, such as serum, plasma, erythropoietin (EPO), insulin, holo transferrin and/or heparin.

In some embodiments, CD34⁺ cells may be cultured in expansion medium for any suitable period of time to obtain a desired number of cells, which can then be cultured in one or more differentiation medium (e.g., to generate enucleated blood cells). In some embodiments, CD34⁺ cells are cultured in expansion medium from 1 day to 10 days. In some embodiments, CD34⁺ cells are cultured in expansion medium for 1, 2, 3, 4, 5, 6, 7, 8, 9, 10, 15, 20, or more days.

### Multiple Differentiation Stages

The in vitro culturing process described herein involves multiple differentiation stages (3, 4, 5, 6, 7 or more), in which CD34⁺ progenitor cells differentiate into mature enucleated red blood cells. In each differentiation stage, CD34⁺ progenitor cells (either obtained from the expansion stage or collected from the original source) or cells obtained from a preceding differentiation stage can be cultured in a medium comprising one or more suitable cytokines (e.g., those described herein) under suitable conditions for a suitable period of time. Biological properties of the cells, such as cell size and expression of surface markers, may be monitored during the course or at the end of each differentiation stage to evaluate the status of erythropoiesis. Whenever necessary, cytokines can be timely supplied and/or withdrawn at each differentiation stage to achieve optimal erythroid differentiation and/or synchronizing the cell population in culture. In some embodiments, the in vitro culturing process described herein involves at least 5 differentiation stages (e.g., 5, 6, 7, or 8 differentiation stages).

At each of the differentiation stages, CD34⁺ progenitor cells, either obtained from the expansion stage described herein or isolated from an original source (e.g., human peripheral blood), or cells obtained from the preceding differentiation stage can be cultured in a suitable medium, such as those described above, supplemented with one or more supplements (e.g., any of the supplements described above) under suitable culturing conditions for a suitable period of time. In some embodiments, one or more of the differentiation media comprise any of the base media provided herein (e.g., Serum-Free Expansion Media, SFEM or SFEM II), Dulbecco's MEM (DMEM), IMDM, DMEM/F12, MEM, Opti-MEM, ISCOVE, HAM F12, HAM F10, M199, L15, 6M NCTC109 medium, Fischer medium, Waymouth medium, VPSFM medium, Williams medium, X-Vivo 15 (e.g., serum-depleted), RPMI (e.g., RPMI-1640)). In some embodiments, one or more of the differentiation media comprise IMDM.

In some embodiments, one or more of the differentiation media is supplemented with holo transferrin (e.g., holo human transferrin) and/or insulin at suitable concentrations. For example, the concentration of holo transferrin in one or more of the differentiation media (e.g., differentiation medium I, II, III, IV, or IV) ranges from 50 μg/ml-1,500 μg/ml (e.g., 150-1,000 μg/ml; 200-500 μg/ml, about 200 μg/ml or about 500 μg/ml). In some embodiments, the concentration of holo transferrin in one or more of the differentiation media is from 50 μg/ml-100 μg/ml, from 50 μg/ml-200 μg/ml, from 50 μg/ml-500 μg/ml, from 50 μg/ml-1000 μg/ml, from 50 μg/ml-1500 μg/ml, from 100 μg/ml-200 μg/ml, from 100 μg/ml-500 μg/ml, from 100 μg/ml-1000 μg/ml, from 100 μg/ml-1500 μg/ml, from 200 μg/ml-500 μg/ml, from 200 μg/ml-1000 μg/ml, from 200 μg/ml-1500 μg/ml, from 500 μg/ml-1000 μg/ml, from 500 μg/ml-1500 μg/ml, or from 1000 μg/ml-1500 μg/ml of holo transferrin. In some embodiments, an amount of holo transferrin in one differentiation medium, (e.g., differentiation III medium) is increased in a subsequent differentiation medium (e.g., differentiation IV medium). In some embodiments, an amount of holo transferrin in one differentiation medium is increased by at least 10%, 15%, 20%, 30%, 40%, 50%, 60%, 70%, 80%, 100%, 120%, 150%, 200%, 300%, 400%, or 500% in a subsequent differentiation medium. In some embodiments, an amount of holo transferrin in one differentiation medium is increased by at least 10 μg/ml, 50 μg/ml, 100 μg/ml, 150 μg/ml, 200 μg/ml, 250 μg/ml, 300 μg/ml, 500 μg/ml, or 1000 μg/ml in a subsequent differentiation medium. In some embodiments, an amount of holo transferrin in one differentiation medium, (e.g., differentiation III medium) is increased from a range of 150 μg/ml-250 μg/ml to a range of 450 μg/ml to 550 μg/ml in a subsequent differentiation medium (e.g., differentiation IV medium).

In some embodiments, one or more of the differentiation media is supplemented with insulin and/or holo transferrin at suitable concentrations. For example, the concentration of insulin can range from 5-20 μg/ml (e.g., 5-15 μg/ml, 5-10 μg/ml, or 10-20 μg/ml). In some embodiments, the concentration of insulin in one or more of the differentiation media is from 1 μg/ml-5 μg/ml, from 1 μg/ml-10 μg/ml, from 1 μg/ml-15 μg/ml, from 1 μg/ml-20 μg/ml, from 1 μg/ml-50 μg/ml, from 1 μg/ml-100 μg/ml, from 5 μg/ml-10 μg/ml, from 5 μg/ml-15 μg/ml, from 5 μg/ml-20 μg/ml, from 5 μg/ml-50 μg/ml, from 5 μg/ml-100 μg/ml, from 10 μg/ml-15 μg/ml, from 10 μg/ml-20 μg/ml, from 10 μg/ml-50 μg/ml, from 10 μg/ml-100 μg/ml, from 15 μg/ml-20 μg/ml, from 15 μg/ml-50 μg/ml, from 15 μg/ml-100 μg/ml, from 20 μg/ml-50 μg/ml, from 20 μg/ml-100 μg/ml, or from 50 μg/ml-100 μg/ml of insulin. In some embodiments, an amount of insulin in one differentiation medium is the same as an amount of insulin in a subsequent differentiation medium. In some embodiments, the amount of insulin is the same or about the same in all differentiation media (e.g., differentiation medium I, II, III, IV, V, or more).

It should be appreciated that any of the differentiation media may also be supplemented with other components commonly used in cell culture, e.g., serum (e.g., human serum), plasma (e.g., human plasma), glutamine, bovine serum albumin, one or more antibiotics (e.g., penicillin and streptomycin), or any combination thereof. In some embodiments, any or all of the differentiation media are supplemented with serum (e.g., serum from a horse, pig, rabbit, goat, cow or human). The amount of serum may be in any suitable amount to support the growth and/or differentiation of CD34⁺ cells in culture. The amount of serum used in one or more of the differentiation media may be any of the amounts provided herein. In some embodiments, any or all of the differentiation media are supplemented with plasma (e.g., plasma from a horse, pig, rabbit, goat, cow or human). The amount of plasma may be in any suitable amount to support the growth and/or differentiation of CD34⁺ cells in culture. The amount of plasma used in one or more of the differentiation media may be any of the amounts provided herein.

In some embodiments, the in vitro culturing process described herein includes at least five differentiation stages, Differentiation stage I (“Dif. I”), Differentiation stage II (“Dif. II”), Differentiation stage III (“Dif. III”), Differentiation stage IV (“Dif. IV”), and Differentiation stage V (“Dif. V”). For each differentiation stage cells are cultured in a corresponding differentiation medium, differentiation medium I, differentiation medium II, differentiation medium III, differentiation medium IV, and differentiation medium V.

### Differentiation I

In Dif. I, the cells may be cultured (e.g., in differentiation medium I) in the presence of a mixture of supplements (e.g., cytokines) including, but not limited to insulin, IL-3, SCF, and EPO (including human EPO, EPO from other species, or EPO analogs such as Epoetin alfa, Epoetin beta, or Darbepoetin alfa) at suitable concentrations for a suitable period of time (e.g., 2-7 days, 4-6 days, 5-7 days, or 2-5 days).

In some embodiments, differentiation medium I comprises IL-3. In some embodiments, the concentration of IL-3 in the medium ranges from 0.1-10 ng/ml (e.g., 0.1-5 ng/ml, 0.5-2 ng/ml, or 0.5-1.5 ng/ml). In some embodiments, the concentration of IL-3 ranges from 0.1 ng/ml to 0.5 ng/ml, from 0.1 ng/ml to 1 ng/ml, from 0.1 ng/ml to 2 ng/ml, from 0.1 ng/ml to 3 ng/ml, from 0.1 ng/ml to 5 ng/ml, from 0.1 ng/ml to 10 ng/ml, from 0.5 ng/ml to 1 ng/ml, from 0.5 ng/ml to 2 ng/ml, from 0.5 ng/ml to 3 ng/ml, from 0.5 ng/ml to 5 ng/ml, from 0.5 ng/ml to 10 ng/ml, from 1 ng/ml to 2 ng/ml, from 1 ng/ml to 3 ng/ml, from 1 ng/ml to 5 ng/ml, from 1 ng/ml to 10 ng/ml, from 2 ng/ml to 3 ng/ml, from 2 ng/ml to 5 ng/ml, from 2 ng/ml to 10 ng/ml, from 3 ng/ml to 5 ng/ml, from 3 ng/ml to 10 ng/ml, or from 5 ng/ml to 10 ng/ml of IL-3. In some embodiments, the concentration of IL-3 ranges from 0.5 ng/ml to 2 ng/ml. It should be appreciated that the amount of IL-3 may be decreased relative to an amount of IL-3 in a previous cell culture step, for example cells grown in any of the expansion media described herein. In some embodiments, the level of IL-3 is decreased as compared to the level of IL-3 in an expansion medium that the cells were grown in prior to (e.g., immediately prior to) culturing the cells in differentiation medium I. In some embodiments, the level of IL-3 is decreased by up to 5%, 10%, 15%, 20%, 30%, 40%, 50%, 60%, 70%, 80%, 90%, 95%, or 99% as compared to a level of IL-3 in an expansion medium that the cells were grown in prior to (e.g., immediately prior to) culturing the cells in differentiation medium I. In some embodiments, the level of IL-3 is decreased by up to 1 ng/ml, 2 ng/ml, 3 ng/ml, 4 ng/ml, 5 ng/ml, 6 ng/ml, 7 ng/ml, 8 ng/ml, 9 ng/ml, 10 ng/ml, 12 ng/ml, 14 ng/ml, 16 ng/ml, 18 ng/ml, 20 ng/ml, 25 ng/ml, 30 ng/ml, 35 ng/ml, or 40 ng/ml as compared to a level of IL-3 in an expansion medium that the cells were grown in prior to (e.g., immediately prior to) culturing the cells in differentiation medium I.

In some embodiments, differentiation medium I comprises stem cell factor (SCF). In some embodiments, the concentration of SCF in the medium ranges from 1-100 ng/ml (e.g., 1-50 ng/ml, 1-22 ng/ml, or 5-15 ng/ml). In some embodiments, the concentration of SCF ranges from 1 ng/ml to 5 ng/ml, from 1 ng/ml to 10 ng/ml, from 1 ng/ml to 15 ng/ml, from 1 ng/ml to 20 ng/ml, from 1 ng/ml to 50 ng/ml, from 1 ng/ml to 1000 ng/ml, from 5 ng/ml to 10 ng/ml, from 5 ng/ml to 15 ng/ml, from 5 ng/ml to 2 ng/ml, from 5 ng/ml to 50 ng/ml, from 5 ng/ml to 100 ng/ml, from 10 ng/ml to 5 ng/ml, from 10 ng/ml to 20 ng/ml, from 10 ng/ml to 50 ng/ml, from 10 ng/ml to 100 ng/ml, from 15 ng/ml to 20 ng/ml, from 15 ng/ml to 50 ng/ml, from 15 ng/ml to 100 ng/ml, from 20 ng/ml to 50 ng/ml, from 20 ng/ml to 100 ng/ml, or from 50 ng/ml to 100 ng/ml of SCF. In some embodiments, the concentration of SCF ranges from 5 ng/ml to 20 ng/ml in the medium.

It should be appreciated that the amount of SCF in the differentiation medium I may be decreased relative to an amount of SCF in a previous cell culture step, for example cells grown in any of the expansion media described herein. In some embodiments, the level of SCF is decreased as compared to the level of SCF in an expansion medium that the cells were grown in prior to (e.g., immediately prior to) culturing the cells in differentiation medium I. In some embodiments, the level of SCF is decreased by up to 5%, 10%, 15%, 20%, 30%, 40%, 50%, 60%, 70%, 80%, 90%, 95%, or 99% as compared to a level of SCF in an expansion medium that the cells were grown in prior to (e.g., immediately prior to) culturing the cells in differentiation medium I. In some embodiments, the level of SCF is decreased by up to 1 ng/ml, 2 ng/ml, 3 ng/ml, 4 ng/ml, 5 ng/ml, 6 ng/ml, 7 ng/ml, 8 ng/ml, 9 ng/ml, 10 ng/ml, 12 ng/ml, 14 ng/ml, 16 ng/ml, 18 ng/ml, 20 ng/ml, 25 ng/ml, 30 ng/ml, 35 ng/ml, or 40 ng/ml as compared to a level of SCF in an expansion medium that the cells were grown in prior to (e.g., immediately prior to) culturing the cells in differentiation medium I.

In some embodiments, differentiation medium I comprises erythropoietin (EPO) or an EPO analog, such as Epoetin alfa, Epoetin beta, or Darbepoetin alfa, which similarly affects erythropoiesis. Accordingly, EPO may be any or an analog of EPO having similar activity thereof. In some embodiments, the EPO is from a horse, pig, rabbit, goat, cow or human. In some embodiments, the EPO is from a human. In some embodiments, the EPO is recombinant EPO, for example recombinant human EPO. In some embodiments, the amount of EPO in the medium ranges from 0.1-40 units/ml (U/ml) (e.g., 0.1-20 U/ml, 0.1-15 U/ml, 0.5-15 U/ml, or 2-5 U/ml). As well known in the art, one EPO unit elicits the same erythropoiesis stimulating response in rodents (historically: fasted rats) as five micromoles of cobaltous chloride. See, e.g., Jelkmann, Nephrol Dial. Transplant, 2009. In some embodiments, the amount of EPO in the medium ranges from 0.1 U/ml to 0.5 U/ml, from 0.1 U/ml to 1 U/ml, from 0.1 U/ml to 5 U/ml, from 0.1 U/ml to 10 U/ml, from 0.1 U/ml to 15 U/ml, from 0.1 U/ml to 20 U/ml, from 0.1 U/ml to 40 U/ml, from 0.5 U/ml to 1 U/ml, from 0.5 U/ml to 5 U/ml, from 0.5 U/ml to 10 U/ml, from 0.5 U/ml to 15 U/ml, from 0.5 U/ml to 20 U/ml, from 0.5 U/ml to 40 U/ml, from 1 U/ml to 5 U/ml, from 1 U/ml to 10 U/ml, from 1 U/ml to 15 U/ml, from 1 U/ml to 20 U/ml, from 1 U/ml to 40 U/ml, from 5 U/ml to 10 U/ml, from 5 U/ml to 15 U/ml, from 5 U/ml to 20 U/ml, from 5 U/ml to 40 U/ml, from 10 U/ml to 15 U/ml, from 10 U/ml to 20 U/ml, from 10 U/ml to 40 U/ml, from 15 U/ml to 20 U/ml, from 15 U/ml to 40 U/ml, or from 20 U/ml to 40 U/ml of EPO. In some embodiments, the amount of EPO in the medium ranges from 2 U/ml to 4 U/ml (e.g., 3 U/ml).

In some embodiments, the differentiation medium I used in Dif. I contains holo human transferrin, insulin, IL-3, SCF, and EPO. In some embodiments, the differentiation medium I used in Dif. I contains from 100 μg/ml to 300 μg/ml holo human transferrin, from 5 μg/ml to 20 μg/ml insulin, from 0.5 ng/ml to 2 ng/ml IL-3, from 5 ng/ml to 20 ng/ml SCF, and from 2 U/ml to 4 U/ml EPO. In some embodiments, differentiation medium I is substantially free of certain other supplements, such as Flt-3 ligand, IL-6, and/or dexamethasone.

In some embodiments, CD34⁺ cells may be cultured in differentiation medium I for any suitable period of time, which can then be cultured in one or more additional differentiation media (e.g., to generate enucleated blood cells). In some embodiments, CD34⁺ cells are cultured in differentiation medium I from 1 day to 10 days. In some embodiments, CD34⁺ cells are cultured in differentiation medium I for 1, 2, 3, 4, 5, 6, 7, 8, 9, 10, 15, 20, or more days. In some embodiments, CD34⁺ cells are cultured in differentiation medium I for 3, 4, or 5 days.

In some embodiments, CD34⁺ cells may be cultured in differentiation medium I at a suitable density for differentiating one or more of the CD34⁺ cells into enucleated erythrocytes. In some embodiments, the cell density of the cultured population of CD34⁺ cells in differentiation medium I can be from about 1×10² cells to about 1×10⁷ cells/mL. In some embodiments, the density of the cultured population of CD34⁺ cells is from 1×10² to 1×10⁷ cells/mL. In some embodiments, the density of the cultured population of CD34⁺ cells is from 1×10² to 1×10³ cells/mL, from 1×10² to 1×10⁴ cells/mL, from 1×10² to 1×10⁵ cells/mL, from 1×10² to 1×10⁶ cells/mL, from 1×10³ to 1×10⁴ cells/mL, from 1×10³ to 1×10⁵ cells/mL, from 1×10³ to 1×10⁶ cells/mL, from 1×10³ to 1×10⁷ cells/mL, from 1×10⁴ to 1×10⁵ cells/mL, from 1×10⁴ to 1×10⁶ cells/mL, from 1×10⁴ to 1×10⁷ cells/mL, from 1×10⁵ to 1×10⁶ cells/mL, from 1×10⁵ to 1×10⁷ cells/mL, or from 1×10⁶ to 1×10⁷ cells/mL. Preferably, the cell density can be from about 50,000 cells/mL to about 200,000 cells/mL (e.g., about 100,000 cells/mL).

### Differentiation II

In Dif. II, the cells may be cultured (e.g., in differentiation medium II) in the presence of a mixture of supplements (e.g., cytokines) including, but not limited to insulin, IL-3, SCF, and EPO (including human EPO, EPO from other species, or EPO analogs such as Epoetin alfa, Epoetin beta, or Darbepoetin alfa) at suitable concentrations for a suitable period of time (e.g., 2-7 days, 4-6 days, 5-7 days, or 2-5 days). It should be appreciated that differentiation medium II may be the same as any of the differentiation media I described above. Accordingly, in some embodiments, differentiation medium I and differentiation medium II are the same. In some embodiments, differentiation medium I and differentiation medium II are different.

In some embodiments, the differentiation medium II used in Dif. II contains holo human transferrin, insulin, IL-3, SCF, and EPO. In some embodiments, the differentiation medium II used in Dif. II contains from 100 μg/ml to 300 μg/ml holo human transferrin, from 5 μg/ml to 20 μg/ml insulin, from 0.5 ng/ml to 2 ng/ml IL-3, from 5 ng/ml to 20 ng/ml SCF, and from 2 U/ml to 4 U/ml EPO. In some embodiments, differentiation medium I is substantially free of certain other supplements, such as Flt-3 ligand, IL-6, and/or dexamethasone.

In some embodiments, CD34⁺ cells may be cultured in differentiation medium II for any suitable period of time, which can then be cultured in one or more additional differentiation media (e.g., to generate enucleated blood cells). In some embodiments, CD34⁺ cells are cultured in differentiation medium II from 1 day to 10 days. In some embodiments, CD34⁺ cells are cultured in differentiation medium II for 1, 2, 3, 4, 5, 6, 7, 8, 9, 10, 15, 20, or more days. In some embodiments, CD34⁺ cells are cultured in differentiation medium II for 3, 4, or 5 days. In some embodiments, CD34⁺ cells are cultured in differentiation medium II for a period of time that is less than the period of time that the cells were cultured (e.g., immediately prior to) in a previous differentiation medium (e.g., differentiation medium I). In some embodiments, CD34⁺ cells are cultured in differentiation medium II for a period of time that is at 1, 2, 3, 4, 5, 6 7, 8, 9, 10, or more days less than the period of time that the cells were cultured (e.g., immediately prior to) in a previous differentiation medium (e.g., differentiation medium I). In some embodiments, CD34⁺ cells are cultured in differentiation medium II for a period of time that is 1, 2, or 3 days less than the period of time that the cells were cultured (e.g., immediately prior to) in a previous differentiation medium (e.g., differentiation medium I).

In some embodiments, CD34⁺ cells may be cultured in differentiation medium II at a suitable density for differentiating one or more of the CD34⁺ cells into enucleated erythrocytes. In some embodiments, The cell density of the cultured population of CD34⁺ cells in differentiation medium II can be from about 1×10² cells to about 1×10⁷ cells/mL. In some embodiments, the density of the cultured population of CD34⁺ cells is from 1×10² to 1×10⁷ cells/mL. In some embodiments, the density of the cultured population of CD34⁺ cells is from 1×10² to 1×10³ cells/mL, from 1×10² to 1×10⁴ cells/mL, from 1×10² to 1×10⁵ cells/mL, from 1×10² to 1×10⁶ cells/mL, from 1×10³ to 1×10⁴ cells/mL, from 1×10³ to 1×10⁵ cells/mL, from 1×10³ to 1×10⁶ cells/mL, from 1×10³ to 1×10⁷ cells/mL, from 1×10⁴ to 1×10⁵ cells/mL, from 1×10⁴ to 1×10⁶ cells/mL, from 1×10⁴ to 1×10⁷ cells/mL, from 1×10⁵ to 1×10⁶ cells/mL, from 1×10⁵ to 1×10⁷ cells/mL, or from 1×10⁶ to 1×10⁷ cells/mL. In some embodiments, the cell density can be from about 100,000 cells/mL to about 500,000 cells/mL (e.g., about 300,000 or 400,000 cells/mL). Preferably, the cell density can be from about 300,000 cells/mL to about 400,000 cells/mL (e.g., about 350,000 cells/mL). In some embodiments, the density of the cultured population of CD34⁺ cells in differentiation medium II is greater than the density of cells cultured (e.g., immediately prior to) in a previous differentiation medium (e.g., differentiation medium I). In some embodiments, the density of the cultured population of CD34⁺ cells in differentiation medium II is up to 10%, 15%, 20%, 30%, 40%, 50%, 60%, 70%, 80%, 100%, 120%, 150%, 200%, 300%, 400%, or 500% greater than the density of cells cultured (e.g., immediately prior to) in a previous differentiation medium (e.g., differentiation medium I). In some embodiments, the density of the cultured population of CD34⁺ cells in differentiation medium II is up to 10,000 cells/mL, 25,000 cells/mL, 100,000 cells/mL, 150,000 cells/mL, 200,000 cells/mL, or 300,000 cells/mL, greater than the density of cells cultured (e.g., immediately prior to) in a previous differentiation medium (e.g., differentiation medium I).

In some embodiments, the density of the cultured population of CD34⁺ cells in differentiation medium II is less than the density of cells cultured (e.g., immediately prior to) in a previous differentiation medium (e.g., differentiation medium I). In some embodiments, the density of the cultured population of CD34⁺ cells in differentiation medium II is diluted by at least 1×, 1.5×, 2×, 2.5×, 3×, 3.5×, 4×, 4.5×, 5×, 5.5×, 6×, 6.5×, 7×, 8×, 9× or at least 10× (e.g., about 4×) relative to the density of cells cultured (e.g., immediately prior to) in a previous differentiation medium (e.g., differentiation medium I). In some embodiments, the density of the cultured population of CD34⁺ cells in differentiation medium II is up to 10,000 cells/mL, 25,000 cells/mL, 100,000 cells/mL, 150,000 cells/mL, 200,000 cells/mL, or 300,000 cells/mL, less than the density of cells cultured (e.g., immediately prior to) in a previous differentiation medium (e.g., differentiation medium I).

### Differentiation III

In Dif. III, the cells may be cultured (e.g., in differentiation medium III) in the presence of a mixture of supplements (e.g., cytokines) including, but not limited to, insulin, SCF, and EPO (including human EPO, EPO from other species, or EPO analogs such as Epoetin alfa, Epoetin beta, or Darbepoetin alfa) at suitable concentrations for a suitable period of time (e.g., 2-7 days, 4-6 days, 5-7 days, or 2-5 days). It should be appreciated that differentiation medium III may contain one, more than one, or all of the same components or supplements as any of the differentiation media I or II described above, with the exception of the amount of IL-3 and/or erythropoietin (EPO). Accordingly, in some embodiments, differentiation medium I and II are different from differentiation medium III. In some embodiments, differentiation medium III comprises a lower amount of EPO than an amount of EPO in a differentiation medium (e.g., differentiation medium II) that the cells have been previously cultured (e.g., immediately prior to) in. In some embodiments, differentiation medium III comprises a lower amount of IL-3 than an amount of IL-3 in a differentiation medium (e.g., differentiation medium II) that the cells have been previously cultured (e.g., immediately prior to) in. In some embodiments, IL-3 is absent from differentiation medium III.

In some embodiments, differentiation medium III comprises erythropoietin (EPO) or an EPO analog, such as Epoetin alfa, Epoetin beta, or Darbepoetin alfa, which similarly affects erythropoiesis. Accordingly, EPO may be any or an analog of EPO having similar activity thereof. In some embodiments, the EPO is from a horse, pig, rabbit, goat, cow or human. In some embodiments, the EPO is from a human. In some embodiments, the EPO is recombinant EPO, for example recombinant human EPO. In some embodiments, the amount of EPO in the medium ranges from 0.1 U/ml-10 U/ml (e.g., 0.1-8 U/ml, 0.1-5 U/ml, 0.5-2 U/ml, or 0.8-1.2 U/ml). In some embodiments, the amount of EPO in the medium ranges from 0.1 U/ml to 0.5 U/ml, from 0.1 U/ml to 1 U/ml, from 0.1 U/ml to 2 U/ml, from 0.1 U/ml to 5 U/ml, from 0.1 U/ml to 10 U/ml, from 0.5 U/ml to 1 U/ml, from 0.5 U/ml to 2 U/ml, from 0.5 U/ml to 5 U/ml, from 0.5 U/ml to 10 U/ml, from 1 U/ml to 2 U/ml, from 1 U/ml to 5 U/ml, from 1 U/ml to 10 U/ml, from 2 U/ml to 5 U/ml, from 2 U/ml to 10 U/ml, or from 5 U/ml to 10 U/ml of EPO. In some embodiments, the amount of EPO in the medium ranges from 0.5 U/ml to 2 U/ml (e.g., 1 U/ml).

It should be appreciated that the amount of EPO in differentiation medium III may be decreased relative to an amount of EPO in a previous cell culture step, for example cells grown in any of the differentiation media (e.g., differentiation medium II) described herein. In some embodiments, the level of EPO is decreased as compared to the level of EPO in a differentiation medium that the cells were grown in prior to (e.g., immediately prior to) culturing the cells in differentiation medium III (e.g., differentiation medium II). In some embodiments, the level of EPO is decreased by up to 5%, 10%, 15%, 20%, 30%, 40%, 50%, 60%, 70%, 80%, 90%, 95%, or 99% as compared to a level of EPO in a differentiation medium that the cells were grown in prior to (e.g., immediately prior to) culturing the cells in differentiation medium III (e.g., differentiation medium II). In some embodiments, the level of EPO is decreased by up to 0.1 U/ml, 0.2 U/ml, 0.3 U/ml, 0.4 U/ml, 0.5 U/ml, 1 U/ml, 1.5 U/ml, 2 U/ml, 2.5 U/ml, 3 U/ml, 4 U/ml, 5 U/ml, 6 U/ml, 7 U/ml, 8 U/ml, 10 U/ml, 15 U/ml, or 20 U/ml as compared to a level of EPO in a differentiation medium that the cells were grown in prior to (e.g., immediately prior to) culturing the cells in differentiation medium III (e.g., differentiation medium II).

It should be appreciated that the amount of IL-3 in differentiation medium III may be decreased relative to an amount of IL-3 in a previous cell culture step, for example cells grown in any of the differentiation media (e.g., differentiation medium II) described herein. In some embodiments, the level of IL-3 is decreased as compared to the level of IL-3 in a differentiation medium that the cells were grown in prior to (e.g., immediately prior to) culturing the cells in differentiation medium III (e.g., differentiation medium II). In some embodiments, the level of IL-3 is decreased by up to 5%, 10%, 15%, 20%, 30%, 40%, 50%, 60%, 70%, 80%, 90%, 95%, 99%, or 100% as compared to a level of IL-3 in a differentiation medium that the cells were grown in prior to (e.g., immediately prior to) culturing the cells in differentiation medium III (e.g., differentiation medium II). In some embodiments, the level of IL-3 is decreased by up to 1 ng/ml, 2 ng/ml, 3 ng/ml, 4 ng/ml, 5 ng/ml, 6 ng/ml, 7 ng/ml, 8 ng/ml, 9 ng/ml, 10 ng/ml, 12 ng/ml, 14 ng/ml, 16 ng/ml, 18 ng/ml, 20 ng/ml, 25 ng/ml, 30 ng/ml, 35 ng/ml, or 40 ng/ml as compared to a level of IL-3 in a differentiation medium that the cells were grown in prior to (e.g., immediately prior to) culturing the cells in differentiation medium III (e.g., differentiation medium II).

In some embodiments, the differentiation medium III used in Dif. III contains holo human transferrin, insulin, SCF, and EPO. In some embodiments, the differentiation medium III used in Dif. III contains from 100 μg/ml to 300 μg/ml holo human transferrin, from 5 μg/ml to 20 μg/ml insulin, from 5 ng/ml to 20 ng/ml SCF, and from 0.1 U/ml to 2 U/ml EPO. In some embodiments, differentiation medium III is substantially free of certain other supplements, such as Flt-3 ligand, IL-6, IL-3 and/or dexamethasone.

In some embodiments, CD34⁺ cells may be cultured in differentiation medium III for any suitable period of time, which can then be cultured in one or more additional differentiation media (e.g., to generate enucleated blood cells). In some embodiments, CD34⁺ cells are cultured in differentiation medium III from 1 day to 12 days. In some embodiments, CD34⁺ cells are cultured in differentiation medium III for 1, 2, 3, 4, 5, 6, 7, 8, 9, 10, 15, 20, or more days. In some embodiments, CD34⁺ cells are cultured in differentiation medium III for 3, 4, or 5 days. In some embodiments, CD34⁺ cells are cultured in differentiation medium III for a period of time that is less than the period of time that the cells were cultured (e.g., immediately prior to) in a previous differentiation medium (e.g., differentiation medium II). In some embodiments, CD34⁺ cells are cultured in differentiation medium III for a period of time that is at 1, 2, 3, 4, 5, 6 7, 8, 9, 10, or more days less than the period of time that the cells were cultured (e.g., immediately prior to) in a previous differentiation medium (e.g., differentiation medium II). In some embodiments, CD34⁺ cells are cultured in differentiation medium III for a period of time that is 1, 2, or 3 days less than the period of time that the cells were cultured (e.g., immediately prior to) in a previous differentiation medium (e.g., differentiation medium II).

In some embodiments, CD34⁺ cells may be cultured in differentiation medium III at a suitable density for differentiating one or more of the CD34⁺ cells into enucleated erythrocytes. In some embodiments, the cell density of the cultured population of CD34⁺ cells in differentiation medium III can be from about 1×10² cells to about 1×10⁷ cells/mL. In some embodiments, the density of the cultured population of CD34⁺ cells is from 1×10² to 1×10⁷ cells/mL. In some embodiments, the density of the cultured population of CD34⁺ cells is from 1×10² to 1×10³ cells/mL, from 1×10² to 1×10⁴ cells/mL, from 1×10² to 1×10⁵ cells/mL, from 1×10² to 1×10⁶ cells/mL, from 1×10³ to 1×10⁴ cells/mL, from 1×10³ to 1×10⁵ cells/mL, from 1×10³ to 1×10⁶ cells/mL, from 1×10³ to 1×10⁷ cells/mL, from 1×10⁴ to 1×10⁵ cells/mL, from 1×10⁴ to 1×10⁶ cells/mL, from 1×10⁴ to 1×10⁷ cells/mL, from 1×10⁵ to 1×10⁶ cells/mL, from 1×10⁵ to 1×10⁷ cells/mL, or from 1×10⁶ to 1×10⁷ cells/mL. Preferably, the cell density can be from about 50,000 cells/mL to about 200,000 cells/mL (e.g., about 100,000 cells/mL). In some embodiments, the density of the cultured population of CD34⁺ cells in differentiation medium III is less than the density of cells cultured (e.g., immediately prior to) in a previous differentiation medium (e.g., differentiation medium II). In some embodiments, the density of the cultured population of CD34⁺ cells in differentiation medium III is decreased by up to 5%, 10%, 15%, 20%, 30%, 40%, 50%, 60%, 70%, 80%, 90%, 95%, or 99% as compared to the density of cells cultured (e.g., immediately prior to) in a previous differentiation medium (e.g., differentiation medium II). In some embodiments, the density of the cultured population of CD34⁺ cells in differentiation medium III is decreased up to 10,000 cells/mL, 25,000 cells/mL, 100,000 cells/mL, 150,000 cells/mL, 200,000 cells/mL, or 300,000 cells/mL as compared to the density of cells cultured (e.g., immediately prior to) in a previous differentiation medium (e.g., differentiation medium II).

### Differentiation IV

In Dif. IV, the cells may be cultured (e.g., in differentiation medium IV) in the presence of a mixture of supplements (e.g., cytokines) including, but not limited to, insulin and EPO (including human EPO, EPO from other species, or EPO analogs such as Epoetin alfa, Epoetin beta, or Darbepoetin alfa) at suitable concentrations for a suitable period of time (e.g., 2-7 days, 4-6 days, 5-7 days, or 2-5 days). It should be appreciated that differentiation medium IV may contain one, more than one, or all of the same components or supplements as differentiation media III described above, with the exception of the amount of holo transferrin, SCF and/or erythropoietin (EPO). Accordingly, in some embodiments, differentiation medium III is different from differentiation medium IV. In some embodiments, differentiation medium IV comprises a lower amount of EPO than an amount of EPO in a differentiation medium (e.g., differentiation medium III) that the cells have been previously cultured (e.g., immediately prior to) in. In some embodiments, differentiation medium IV comprises a lower amount of SCF than an amount of SCF in a differentiation medium (e.g., differentiation medium III) that the cells have been previously cultured (e.g., immediately prior to) in. In some embodiments, SCF is absent from differentiation medium IV. In some embodiments, differentiation medium IV comprises a higher amount of holo transferrin than an amount of holo transferrin in a differentiation medium (e.g., differentiation medium III) that the cells have been previously cultured (e.g., immediately prior to) in.

In some embodiments, differentiation medium IV comprises erythropoietin (EPO) or an EPO analog, such as Epoetin alfa, Epoetin beta, or Darbepoetin alfa, which similarly affects erythropoiesis. Accordingly, EPO may be any or an analog of EPO having similar activity thereof. In some embodiments, the EPO is from a horse, pig, rabbit, goat, cow or human. In some embodiments, the EPO is from a human. In some embodiments, the EPO is recombinant EPO, for example recombinant human EPO. In some embodiments, the amount of EPO in the medium ranges from 0.01 U/ml-1 U/ml (e.g., 0.01-0.8 U/ml, 0.01-0.5 U/ml, 0.05-0.2 U/ml, or 0.08-0.12 U/ml). In some embodiments, the amount of EPO in the medium ranges from 0.01 U/ml to 0.05 U/ml, from 0.01 U/ml to 0.1 U/ml, from 0.01 U/ml to 0.2 U/ml, from 0.01 U/ml to 0.5 U/ml, from 0.01 U/ml to 1.0 U/ml, from 0.05 U/ml to 0.1 U/ml, from 0.05 U/ml to 0.2 U/ml, from 0.05 U/ml to 0.5 U/ml, from 0.05 U/ml to 1.0 U/ml, from 0.1 U/ml to 0.2 U/ml, from 0.1 U/ml to 0.5 U/ml, from 0.1 U/ml to 1.0 U/ml, from 0.2 U/ml to 0.5 U/ml, from 0.2 U/ml to 1.0 U/ml, or from 0.5 U/ml to 1.0 U/ml of EPO. In some embodiments, the amount of EPO in the medium ranges from 0.05 U/ml to 0.2 U/ml (e.g., 0.1 U/ml).

It should be appreciated that the amount of EPO in differentiation medium IV may be decreased relative to an amount of EPO in a previous cell culture step, for example cells grown in any of the differentiation media (e.g., differentiation medium III) described herein. In some embodiments, the level of EPO is decreased as compared to the level of EPO in a differentiation medium that the cells were grown in prior to (e.g., immediately prior to) culturing the cells in differentiation medium IV (e.g., differentiation medium III). In some embodiments, the level of EPO is decreased by up to 5%, 10%, 15%, 20%, 30%, 40%, 50%, 60%, 70%, 80%, 90%, 95%, or 99% as compared to a level of EPO in a differentiation medium that the cells were grown in prior to (e.g., immediately prior to) culturing the cells in differentiation medium IV (e.g., differentiation medium III). In some embodiments, the level of EPO is decreased by up to 0.1 U/ml, 0.2 U/ml, 0.3 U/ml, 0.4 U/ml, 0.5 U/ml, 0.6 U/ml, 0.7 U/ml, 0.8 U/ml, 0.9 U/ml, 1 U/ml, 1.5 U/ml, 2 U/ml, 2.5 U/ml, 3 U/ml, 4 U/ml, 5 U/ml, 6 U/ml, 7 U/ml, 8 U/ml, 10 U/ml, 15 U/ml, or 20 U/ml as compared to a level of EPO in a differentiation medium that the cells were grown in prior to (e.g., immediately prior to) culturing the cells in differentiation medium IV (e.g., differentiation medium III).

It should be appreciated that the amount of SCF in differentiation medium IV may be decreased relative to an amount of SCF in a previous cell culture step, for example cells grown in any of the differentiation media (e.g., differentiation medium III) described herein. In some embodiments, the level of SCF is decreased as compared to the level of SCF in a differentiation medium that the cells were grown in prior to (e.g., immediately prior to) culturing the cells in differentiation medium IV (e.g., differentiation medium III). In some embodiments, the level of SCF is decreased by up to 5%, 10%, 15%, 20%, 30%, 40%, 50%, 60%, 70%, 80%, 90%, 95%, 99%, or 100% as compared to a level of SCF in a differentiation medium that the cells were grown in prior to (e.g., immediately prior to) culturing the cells in differentiation medium IV (e.g., differentiation medium III). In some embodiments, the level of SCF is decreased by up to 1 ng/ml, 2 ng/ml, 3 ng/ml, 4 ng/ml, 5 ng/ml, 6 ng/ml, 7 ng/ml, 8 ng/ml, 9 ng/ml, 10 ng/ml, 12 ng/ml, 14 ng/ml, 16 ng/ml, 18 ng/ml, 20 ng/ml, 25 ng/ml, 30 ng/ml, 35 ng/ml, or 40 ng/ml as compared to a level of SCF in a differentiation medium that the cells were grown in prior to (e.g., immediately prior to) culturing the cells in differentiation medium IV (e.g., differentiation medium III). In some embodiments, SCF is absent from differentiation medium IV.

It should be appreciated that the amount of holo transferrin in differentiation medium IV may be increased relative to an amount of holo transferrin in a previous cell culture step, for example cells grown in any of the differentiation media (e.g., differentiation medium III) described herein. In some embodiments, an amount of holo transferrin in differentiation medium IV is increased by at least 10%, 15%, 20%, 30%, 40%, 50%, 60%, 70%, 80%, 100%, 120%, 150%, 200%, 300%, 400%, or 500% as compared to a level of holo transferrin in a differentiation medium that the cells were grown in prior to (e.g., immediately prior to) culturing the cells in differentiation medium IV (e.g., differentiation medium III). In some embodiments, an amount of holo transferrin in differentiation medium IV is increased by at least 10 μg/ml, 50 μg/ml, 100 μg/ml, 150 μg/ml, 200 μg/ml, 250 μg/ml, 300 μg/ml, 500 μg/ml, or 1000 μg/ml as compared to a level of holo transferrin in a differentiation medium that the cells were grown in prior to (e.g., immediately prior to) culturing the cells in differentiation medium IV (e.g., differentiation medium III). In some embodiments, an amount of holo transferrin in one differentiation medium, (e.g., differentiation III medium) is increased from a range of 150 μg/ml-250 μg/ml to a range of 450 μg/ml to 550 μg/ml in a subsequent differentiation medium (e.g., differentiation IV medium).

In some embodiments, the differentiation medium IV used in Dif. IV contains holo human transferrin, insulin, and EPO. In some embodiments, the differentiation medium IV used in Dif. IV contains from 400 μg/ml to 600 μg/ml holo human transferrin, from 5 μg/ml to 20 μg/ml insulin, and from 0.05 U/ml to 0.2 U/ml EPO. It should be appreciated that the amount of EPO in differentiation medium IV may be decreased reletaverelative to an amount of EPO in a previous cell culture step, for example cells grown in any of the differentiation media (e.g., differentiation medium III) described herein. In some embodiments, differentiation medium IV is substantially free of certain other supplements, such as Flt-3 ligand, IL-6, IL-3, SCF and/or dexamethasone.

In some embodiments, CD34⁺ cells may be cultured in differentiation medium IV for any suitable period of time, which can then be cultured in one or more additional differentiation media (e.g., to generate enucleated blood cells). In some embodiments, CD34⁺ cells are cultured in differentiation medium IV from 1 day to 12 days. In some embodiments, CD34⁺ cells are cultured in differentiation medium IV for 1, 2, 3, 4, 5, 6, 7, 8, 9, 10, 15, 20, or more days. In some embodiments, CD34⁺ cells are cultured in differentiation medium IV for 3, 4, or 5 days. In some embodiments, CD34⁺ cells are cultured in differentiation medium IV for a period of time that is less than the period of time (e.g., 1, 2, 3, 4, or days) that the cells were cultured (e.g., immediately prior to) in a previous differentiation medium (e.g., differentiation medium III). In some embodiments, CD34+ cells are cultured in differentiation medium IV for a period of time that is more than the period of time (e.g., 1, 2, 3, 4, or days) that the cells were cultured (e.g., immediately prior to) in a previous differentiation medium (e.g., differentiation medium III). In some embodiments, CD34⁺ cells are cultured in differentiation medium IV for a period of time that is the same as the period of time that the cells were cultured (e.g., immediately prior to) in a previous differentiation medium (e.g., differentiation medium III).

In some embodiments, CD34⁺ cells may be cultured in differentiation medium IV at a suitable density for differentiating one or more of the CD34⁺ cells into enucleated erythrocytes. In some embodiments, the cell density of the cultured population of CD34⁺ cells in differentiation medium IV can be from about 1×10³ cells to about 1×10⁷ cells/mL. In some embodiments, the density of the cultured population of CD34⁺ cells is from 1×10³ to 1×10⁷ cells/mL. In some embodiments, the density of the cultured population of CD34⁺ cells is from 1×10³ to 1×10⁴ cells/mL, from 1×10³ to 1×10⁵ cells/mL, from 1×10³ to 1×10⁶ cells/mL, from 1×10³ to 1×10⁷ cells/mL, from 1×10⁴ to 1×10⁵ cells/mL, from 1×10⁴ to 1×10⁶ cells/mL, from 1×10⁴ to 1×10⁷ cells/mL, from 1×10⁵ to 1×10⁶ cells/mL, from 1×10⁵ to 1×10⁷ cells/mL, or from 1×10⁶ to 1×10⁷ cells/mL. Preferably, the cell density can be from about 500,000 cells/mL to about 1,500,000 cells/mL (e.g., about 1,000,000 cells/mL). In some embodiments, the density of the cultured population of CD34⁺ cells in differentiation medium IV is greater than the density of cells cultured (e.g., immediately prior to) in a previous differentiation medium (e.g., differentiation medium III). In some embodiments, the density of the cultured population of CD34⁺ cells in differentiation medium IV is increased by up to 50%, 100%, 150%, 200%, 300%, 400%, 500%, 600%, 700%, 800%, 900%, 1000%, 1500%, 2000%, 2500%, 3000%, 4000%, or 5000% as compared to the density of cells cultured (e.g., immediately prior to) in a previous differentiation medium (e.g., differentiation medium III). In some embodiments, the density of the cultured population of CD34⁺ cells in differentiation medium IV is increased up to 100,000 cells/mL, 250,000 cells/mL, 1,000,000 cells/mL, 1,500,000 cells/mL, 2,000,000 cells/mL, or 3,000,000 cells/mL as compared to the density of cells cultured (e.g., immediately prior to) in a previous differentiation medium (e.g., differentiation medium III).

### Differentiation V

In Dif. V, the cells may be cultured (e.g., in differentiation medium V) in the presence of a mixture of supplements (e.g., cytokines) including, but not limited to, insulin at suitable concentrations for a suitable period of time (e.g., 2-7 days, 4-6 days, 5-7 days, or 2-5 days). It should be appreciated that differentiation medium V may contain one, more than one, or all of the same components or supplements as differentiation media IV described above, with the exception of the amount of erythropoietin (EPO). Accordingly, in some embodiments, differentiation medium V is different from differentiation medium IV. In some embodiments, differentiation medium V comprises a lower amount of EPO than an amount of EPO in a differentiation medium (e.g., differentiation medium IV) that the cells have been previously cultured (e.g., immediately prior to) in. In some embodiments, EPO is absent from differentiation medium V.

It should be appreciated that the amount of EPO in differentiation medium V may be decreased relative to an amount of EPO in a previous cell culture step, for example cells grown in any of the differentiation media (e.g., differentiation medium IV) described herein. In some embodiments, the level of EPO is decreased as compared to the level of EPO in a differentiation medium that the cells were grown in prior to (e.g., immediately prior to) culturing the cells in differentiation medium V (e.g., differentiation medium IV). In some embodiments, the level of EPO is decreased by up to 5%, 10%, 15%, 20%, 30%, 40%, 50%, 60%, 70%, 80%, 90%, 95%, 99%, or 100% as compared to a level of EPO in a differentiation medium that the cells were grown in prior to (e.g., immediately prior to) culturing the cells in differentiation medium V (e.g., differentiation medium IV). In some embodiments, the level of EPO is decreased by up to 0.01 U/ml, 0.02 U/ml, 0.03 U/ml, 0.04 U/ml, 0.05 U/ml, 0.06 U/ml, 0.07 U/ml, 0.08 U/ml, 0.09 U/ml, 0.1 U/ml, 0.15 U/ml, 0.2 U/ml, 0.25 U/ml, 0.3 U/ml, 0.4 U/ml, 0.5 U/ml, 0.6 U/ml, 0.7 U/ml, 0.8 U/ml, 1.0 U/ml, 1.5 U/ml, or 2.0 U/ml as compared to a level of EPO in a differentiation medium that the cells were grown in prior to (e.g., immediately prior to) culturing the cells in differentiation medium V (e.g., differentiation medium IV).

In some embodiments, the differentiation medium V used in Dif. V contains holo human transferrin, and insulin. In some embodiments, the differentiation medium V used in Dif. V contains from 400 μg/ml to 600 μg/ml holo human transferrin, and from 5 μg/ml to 20 μg/ml insulin. In some embodiments, differentiation medium IV is substantially free of certain other supplements, such as Flt-3 ligand, IL-6, IL-3, SCF, EPO and/or dexamethasone.

In some embodiments, CD34⁺ cells may be cultured in differentiation medium V for any suitable period of time, which can then be cultured in one or more additional differentiation media (e.g., to generate enucleated blood cells). In some embodiments, CD34⁺ cells are cultured in differentiation medium V from 1 day to 12 days. In some embodiments, CD34⁺ cells are cultured in differentiation medium V for 1, 2, 3, 4, 5, 6, 7, 8, 9, 10, 15, 20, or more days. In some embodiments, CD34⁺ cells are cultured in differentiation medium V for 2, 3, 4, or 5 days. In some embodiments, CD34⁺ cells are cultured in differentiation medium V for a period of time that is less than the period of time (e.g., 1, 2, 3, 4, or days) that the cells were cultured (e.g., immediately prior to) in a previous differentiation medium (e.g., differentiation medium IV). In some embodiments, CD34⁺ cells are cultured in differentiation medium V for a period of time that is more than the period of time (e.g., 1, 2, 3, 4, or days) that the cells were cultured (e.g., immediately prior to) in a previous differentiation medium (e.g., differentiation medium IV). In some embodiments, CD34⁺ cells are cultured in differentiation medium V for a period of time that is the same as the period of time that the cells were cultured (e.g., immediately prior to) in a previous differentiation medium (e.g., differentiation medium IV).

In some embodiments, CD34⁺ cells may be cultured in differentiation medium V at a suitable density for differentiating one or more of the CD34⁺ cells into enucleated erythrocytes. In some embodiments, the cell density of the cultured population of CD34⁺ cells in differentiation medium IV can be from about 1×10³ cells to about 1×10⁷ cells/mL. In some embodiments, the density of the cultured population of CD34⁺ cells is from 1×10³ to 1×10⁷ cells/mL. In some embodiments, the density of the cultured population of CD34⁺ cells is from 1×10³ to 1×10⁴ cells/mL, from 1×10³ to 1×10⁵ cells/mL, from 1×10³ to 1×10⁶ cells/mL, from 1×10³ to 1×10⁷ cells/mL, from 1×10⁴ to 1×10⁵ cells/mL, from 1×10⁴ to 1×10⁶ cells/mL, from 1×10⁴ to 1×10⁷ cells/mL, from 1×10⁵ to 1×10⁶ cells/mL, from 1×10⁵ to 1×10⁷ cells/mL, or from 1×10⁶ to 1×10⁷ cells/mL. Preferably, the cell density can be from about 2,000,000 cells/mL to about 8,000,000 cells/mL (e.g., about 5,000,000 cells/mL). In some embodiments, the density of the cultured population of CD34⁺ cells in differentiation medium V is greater than the density of cells cultured (e.g., immediately prior to) in a previous differentiation medium (e.g., differentiation medium IV). In some embodiments, the density of the cultured population of CD34⁺ cells in differentiation medium V is increased by up to 50%, 100%, 150%, 200%, 300%, 400%, 500%, 600%, 700%, 800%, 900%, 1000%, 1500%, 2000%, 2500%, 3000%, 4000%, or 5000% as compared to the density of cells cultured (e.g., immediately prior to) in a previous differentiation medium (e.g., differentiation medium IV). In some embodiments, the density of the cultured population of CD34⁺ cells in differentiation medium V is increased up to 500,000 cells/mL, 1,000,000 cells/mL, 2,000,000 cells/mL, 4,000,000 cells/mL, 5,000,000 cells/mL, or 6,000,000 cells/mL, 7,000,000 cells/mL, 8,000,000 cells/mL, or 9,000,000 cells/mL as compared to the density of cells cultured (e.g., immediately prior to) in a previous differentiation medium (e.g., differentiation medium IV).

In some embodiments, the in vitro culturing process described herein may include one or any combination of the differentiation stages described herein, for example, Dif. I and Dif. II, Dif. I and Dif. III, Dif. I and Dif. IV, Dif. I and Dif. V, Dif. II and Dif. III, Dif. II and Dif. IV, Dif. II and Dif. V, Dif. III and Dif. IV, Dif. III and Dif. V, or Dif. IV and Dif. V.

Prior to the differentiation stages and/or an expansion stage, the CD34⁺ progenitor cells may be genetically modified such that they express surface proteins of interest, for example, fusion proteins comprising a red blood cell transmembrane protein and an antigen binding protein, which are discussed in detail below.

Some aspects of the disclosure provide an isolated population of red blood cells prepared by any of the methods described herein. In some embodiments, the population of red blood cells are genetically engineered. In some embodiments, the population of red blood cells are genetically engineered to express any of the fusion proteins provided herein. In some embodiments, the disclosure provides an isolated population of red blood cells (e.g., engineered red blood cells), wherein at least 40%, 50%, 60%, 70%, 80% 90%, 95%, or 99% of the red blood cells in the isolated population are enucleated. In some embodiments, the disclosure provides an isolated population of red blood cells (e.g., engineered red blood cells), wherein from 40% to 100% of the red blood cells in the isolated population are enucleated. In some embodiments, the disclosure provides an isolated population of red blood cells (e.g., engineered red blood cells), wherein from 40% to 50%, from 40% to 60%, from 40% to 70%, from 40% to 80%, from 40% to 90%, from 40% to 95%, from 40% to 99%, from 60% to 70%, from 60% to 80%, from 60% to 90%, from 60% to 95%, from 60% to 95%, from 80% to 90%, from 80% to 95%, from 80% to 99%, from 80% to 100%, from 90% to 95%, from 90% to 99%, or from 90% to 100% of the red blood cells in the isolated population are enucleated.

### Fusion Proteins

Some aspects of the disclosure are based, at least in part, on the surprising discovery that fusion proteins comprising a red blood cell transmembrane protein and an antigen binding protein that binds to an epitope of an antigen can be expressed on red cells to neutralize the toxic effects of botulinum toxin (e.g., botuninum toxin types A, B, C, D, E, F or G) in vivo. Accordingly, some aspects of the disclosure provide fusion proteins comprising a red blood cell transmembrane protein and an antigen binding protein that binds to an epitope of an antigen, such as a toxin or an antigen of a pathogen. In some embodiments, the fusion proteins comprises more than one (e.g., two, three, four, five, or more) antigen binding protein, which may bind to the same epitope of different epitopes of an antigen.

The terms “protein,” “peptide” and “polypeptide” are used interchangeably herein, and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof. The term “fusion protein” as used herein refers to a hybrid polypeptide which comprises protein domains from at least two different proteins.

In some embodiments, the disclosure provides fusion proteins that include a red blood cell transmembrane protein and an antigen binding protein, (e.g., a first antigen binding protein) and an antigen binding protein that binds to a first epitope of an antigen (e.g., a first epitope of a first antigen). As used herein, a “red blood cell transmembrane protein” refers to a membrane-bound protein, all or part of which interacts with the hydrophobic core of the phospholipid bilayer of a red blood cell. Accordingly, red blood cell transmembrane proteins include integral membrane proteins that span the entire cell membrane as well as proteins that span at least a portion of the hydrophobic core of the phospholipid bilayer of a red blood cell. In some embodiments, the red blood cell transmembrane protein comprises one or more membrane-spanning domains (e.g., one, two, three, four, five, six, or seven membrane-spanning domains). In some embodiments, the red blood cell transmembrane protein is a naturally-occurring red blood cell transmembrane protein (e.g., from a human, monkey, ape, mouse, rat, cow, dog or pig), or a non-naturally-occurring variant or fragment thereof that interacts with the hydrophobic core of the phospholipid bilayer of a red blood cell.

In some embodiments, the red blood cell transmembrane protein is a type I red blood cell transmembrane protein, such as glycophorin A, intercellular adhesion molecule 4 (ICAM-4), Lutheran glycoprotein (CD329), or Basigin (CD147). Type I transmembrane proteins are single-pass transmembrane proteins which have their N-termini exposed to the extracellular or luminal space. In another example, the red blood cell transmembrane protein is a type II red blood cell transmembrane protein, e.g., Kell, or CD71. Type II transmembrane proteins are single-pass transmembrane proteins which have their C-termini exposed to the extracellular or luminal space. In another example, the cell surface protein is a fusion protein comprising a type III red cell transmembrane protein. Type III membrane proteins are multi-pass structures, which usually have their N-termini exposed to the extracellular or luminal space. Examples of type III red cell transmembrane proteins include, without limitation, GLUT1, Aquaporin 1, and Band 3. In one example, a type III transmembrane protein can be fused with an antigen binding protein at the N-terminus of the transmembrane protein, or at the C-terminus of the transmembrane protein. The red blood cell transmembrane protein can be from any organism including, without limitation, a human, monkey, ape, mouse, rat, cow, dog or pig. In some embodiments, the red blood cell transmembrane protein is from a human.

The proteins useful herein (e.g., red blood cell transmembrane proteins) also include variants or fragments thereof. As used herein, “variant”, refers to a portion of a protein retaining at least one functional i.e. binding or interaction ability and/or therapeutic property thereof. The level or degree of which the property is retained may be reduced relative to the wild type protein but is typically the same or similar in kind. Generally, variants are overall very similar, and, in many regions, identical to the amino acid sequence of the protein described herein.

The variant proteins may comprise, or alternatively consist of, an amino acid sequence which is at least 70%, 75%, 80%, 85%, 90%, 95%, 96%, 97%, 98%, 99% or 100%, identical to, for example, the amino acid sequence of a protein such as a red blood cell transmembrane protein. Further polypeptides encompassed by the invention are polypeptides encoded by polynucleotides which hybridize to the complement of a nucleic acid molecule encoding a protein such as a red blood cell transmembrane protein under stringent hybridization conditions (e.g., hybridization to filter bound DNA in 6× Sodium chloride/Sodium citrate (SSC) at about 45 degrees Celsius, followed by one or more washes in 0.2.times.SSC, 0.1% SDS at about 50-65 degrees Celsius), under highly stringent conditions (e.g., hybridization to filter bound DNA in 6× sodium chloride/Sodium citrate (SSC) at about 45 degrees Celsius, followed by one or more washes in 0.1.times.SSC, 0.2% SDS at about 68 degrees Celsius), or under other stringent hybridization conditions which are known to those of skill in the art (see, for example, Ausubel, F. M. et al., eds., 1989 Current protocol in Molecular Biology, Green publishing associates, Inc., and John Wiley & Sons Inc., New York, at pages 6.3.1-6.3.6 and 2.10.3).

By a polypeptide having an amino acid sequence at least, for example, 95% “identical” to a query amino acid sequence, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence may be inserted, deleted, or substituted with another amino acid. These alterations of the reference sequence may occur at the amino- or carboxy-terminal positions of the reference amino acid sequence or anywhere between those terminal positions, interspersed either individually among residues in the reference sequence or in one or more contiguous groups within the reference sequence.

As a practical matter, whether any particular polypeptide is at least 80%, 85%, 90%, 95%, 96%, 97%, 98% or 99% identical to, for instance, the amino acid sequence of a protein such as a red blood cell transmembrane protein, can be determined conventionally using known computer programs. A preferred method for determining the best overall match between a query sequence (a sequence of the present invention) and a subject sequence, also referred to as a global sequence alignment, can be determined using the FASTDB computer program based on the algorithm of Brutlag et al. (Comp. App. Biosci. 6:237-245 (1990)). In a sequence alignment the query and subject sequences are either both nucleotide sequences or both amino acid sequences. The result of said global sequence alignment is expressed as percent identity. Preferred parameters used in a FASTDB amino acid alignment are: Matrix=PAM 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject amino acid sequence, whichever is shorter.

If the subject sequence is shorter than the query sequence due to N- or C-terminal deletions, not because of internal deletions, a manual correction must be made to the results. This is because the FASTDB program does not account for N- and C-terminal truncations of the subject sequence when calculating global percent identity. For subject sequences truncated at the N- and C-termini, relative to the query sequence, the percent identity is corrected by calculating the number of residues of the query sequence that are N- and C-terminal of the subject sequence, which are not matched/aligned with a corresponding subject residue, as a percent of the total bases of the query sequence. Whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of the present invention. Only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence.

The disclosure also provides non-naturally-occurring variants and fragments of any of the red blood cell transmembrane proteins provided herein that interact with the hydrophobic core of the phospholipid bilayer of a red blood cell. In some embodiments, the red blood cell transmembrane protein comprises an amino acid sequence that is at least 70%, at least 75%, at least 85%, at least 90%, at least 91%, at least 92%, at least 93%, at least 94%, at least 95%, at least 96%, at least 97%, at least 98%, at least 99%, or at least 99.5% identical to a naturally-occurring red blood cell transmembrane protein, or any of the red blood cell transmembrane proteins provided herein (e.g. SEQ ID NOs: 10-12). In some embodiments, the red blood cell transmembrane protein comprises an amino acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more or more mutations compared a naturally-occurring red blood cell transmembrane protein, or any of the red blood cell transmembrane proteins provided herein (e.g. SEQ ID NOs: 10-12). In some embodiments, the red blood cell transmembrane protein comprises an amino acid sequence that has at least 10, at least 15, at least 20, at least 30, at least 40, at least 50, at least 60, at least 70, at least 80, at least 90, at least 100, at least 150, at least 200, at least 250, at least 300, at least 350, at least 400, at least 500, at least 600, at least 700, at least 800, at least 900, at least 1000, at least 1100, or at least 1200 identical contiguous amino acid residues as compared to a naturally-occurring red blood cell transmembrane protein, or any of the red blood cell transmembrane proteins provided herein (e.g. SEQ ID NOs: 10-12).

The disclosure also provides fragments of naturally-occurring red blood cell transmembrane proteins and non-naturally-occurring variants of any of the red blood cell transmembrane proteins provided herein that interact with the hydrophobic core of the phospholipid bilayer of a red blood cell. In some embodiments, the red blood cell transmembrane comprises an amino acid sequence that comprises at least 60%, at least 65%, at least 70%, at least 75%, at least 80%, at least 85%, at least 90%, at least 95%, at least 96%, at least 97%, at least 98%, at least 99%, or at least 99.5% of the amino acid sequence of a naturally-occurring red blood cell transmembrane protein, or any of the red blood cell transmembrane proteins provided herein (e.g. SEQ ID NOs: 10-12). In some embodiments, the red blood cell transmembrane protein is an N-terminal truncation of a naturally-occurring red blood cell transmembrane protein, or any of the red blood cell transmembrane proteins provided herein (e.g. SEQ ID NOs: 10-12). For example, the N-terminal truncation may include the absence of the N-terminal 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 amino acids of a naturally-occurring red blood cell transmembrane protein, or any of the red blood cell transmembrane proteins provided herein (e.g. SEQ ID NOs: 10-12). In some embodiments, the red blood cell transmembrane protein is an C-terminal truncation of a naturally-occurring red blood cell transmembrane protein, or any of the red blood cell transmembrane proteins provided herein (e.g. SEQ ID NOs: 10-12). For example, the C-terminal truncation may include the absence of the C-terminal 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 amino acids of a naturally-occurring red blood cell transmembrane protein, or any of the red blood cell transmembrane proteins provided herein (e.g. SEQ ID NOs: 10-12).

GPA Signal Peptide:
(SEQ ID NO: 9)
MYGKIIFVLLLSEIVSISA
GPA(Absent the signal peptide):
(SEQ ID NO: 10)
LSTTEVAMHTSTSSSVTKSYISSQTNDTHKRDTYAATPRAHEVSEIS
VRTVYPPEEETGERVQLAHHFSEPEITLIIFGVMAGVIGTILLISYG
IRRLIKKSPSDVKPLPSPDTDVPLSSVEIENPETSDQ
GPA(Full Length):
(SEQ ID NO: 11)
MYGKIIFVLLLSEIVSISALSTTEVAMHTSTSSSVTKSYISSQTNDT
HKRDTYAATPRAHEVSEISVRTVYPPEEETGERVQLAHHFSEPEITL
IIFGVMAGVIGTILLISYGIRRLIKKSPSDVKPLPSPDTDVPLSSVE
IENPETSDQ
Kell(Full Length):
(SEQ ID NO: 12)
MEGGDQSEEEPRERSQAGGMGTLWSQESTPEERLPVEGSRPWAVARR
VLTAILILGLLLCFSVLLFYNFQNCGPRPCETSVCLDLRDHYLASGN
TSVAPCTDFFSFACGRAKETNNSFQELATKNKNRLRRILEVQNSWHP
GSGEEKAFQFYNSCMDTLAIEAAGTGPLRQVIEELGGWRISGKWTSL
NFNRTLRLLMSQYGHFPFFRAYLGPHPASPHTPVIQIDQPEFDVPLK
QDQEQKIYAQIFREYLTYLNQLGTLLGGDPSKVQEHSSLSISITSRL
FQFLRPLEQRRAQGKLFQMVTIDQLKEMAPAIDWLSCLQATFTPMSL
SPSQSLVVHDVEYLKNMSQLVEEMLLKQRDFLQSHMILGLVVTLSPA
LDSQFQEARRKLSQKLRELTEQPPMPARPRWMKCVEETGTFFEPTLA
ALFVREAFGPSTRSAAMKLFTAIRDALITRLRNLPWMNEETQNMAQD
KVAQLQVEMGASEWALKPELARQEYNDIQLGSSFLQSVLSCVRSLRA
RIVQSFLQPHPQHRWKVSPWDVNAYYSVSDHVVVFPAGLLQPPFFHP
GYPRAVNFGAAGSIMAHELLHIFYQLLLPGGCLACDNHALQEAHLCL
KRHYAAFPLPSRTSFNDSLTFLENAADVGGLAIALQAYSKRLLRHHG
ETVLPSLDLSPQQIFFRSYAQVMCRKPSPQDSHDTHSPPHLRVHGPL
SSTPAFARYFRCARGALLNPSSRCQLW

In some embodiments, any of the fusion proteins further comprise one or more antigen binding proteins, e.g., antibodies or any antigen binding fragment thereof. An antibody (interchangeably used in plural form) may be an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term “antibody” encompasses not only intact (e.g., full-length) polyclonal or monoclonal antibodies, but also antigen-binding fragments thereof (such as Fab, Fab′, F(ab′)2, Fv), single chain (scFv), mutants thereof, fusion proteins comprising an antibody portion, humanized antibodies, chimeric antibodies, diabodies, linear antibodies, single chain antibodies, multispecific antibodies (e.g., bispecific antibodies) and any other modified configuration of the immunoglobulin molecule that comprises an antigen recognition site of the required specificity, including glycosylation variants of antibodies, amino acid sequence variants of antibodies, and covalently modified antibodies. An antibody includes an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known. Antibodies from mammalian species (e.g., human, mouse, rat, goat, pig, horse, cattle, camel) are within the scope of the term, as are antibodies from non-mammalian species (e.g., from birds, reptiles, amphibia) are also within the scope of the term, e.g., IgY antibodies.

Only part of an antibody is involved in the binding of the antigen, and antigen-binding antibody fragments, their preparation and use, are well known to those of skill in the art. As is well-known in the art, only a small portion of an antibody molecule, the paratope, is involved in the binding of the antibody to its epitope (see, in general, Clark, W. R. (1986) The Experimental Foundations of Modern Immunology Wiley & Sons, Inc., New York; Roitt, I. (1991) Essential Immunology, 7th Ed., Blackwell Scientific Publications, Oxford). Suitable antibodies and antibody fragments for use in the context of some embodiments of the present invention include, for example, human antibodies, humanized antibodies, domain antibodies, F(ab′), F(ab′)2, Fab, Fv, Fc, and Fd fragments, antibodies in which the Fc and/or FR and/or CDR1 and/or CDR2 and/or light chain CDR3 regions have been replaced by homologous human or non-human sequences; antibodies in which the FR and/or CDR1 and/or CDR2 and/or light chain CDR3 regions have been replaced by homologous human or non-human sequences; antibodies in which the FR and/or CDR1 and/or CDR2 and/or light chain CDR3 regions have been replaced by homologous human or non-human sequences; and antibodies in which the FR and/or CDR1 and/or CDR2 regions have been replaced by homologous human or non-human sequences. In some embodiments, so-called single chain antibodies (e.g., ScFv), (single) domain antibodies, and other intracellular antibodies may be used in the context of the present invention. Domain antibodies (single domain antibodies), camelid and camelized antibodies and fragments thereof, for example, VHH domains, or nanobodies, such as those described in patents and published patent applications of Ablynx NV and Domantis are also encompassed in the term antibody. A single domain antibody may consist of a single monomeric variable antibody domain and is capable of specifically binding to an antigen. Further, chimeric antibodies, e.g., antibodies comprising two antigen-binding domains that bind to different antigens, are also suitable for use in the context of some embodiments of the present invention.

The term “antigen-binding antibody fragment,” as used herein, refers to a fragment of an antibody that comprises the paratope, or a fragment of the antibody that binds to the antigen the antibody binds to, with similar specificity and affinity as the intact antibody. Antibodies, e.g., fully human monoclonal antibodies, may be identified using phage display (or other display methods such as yeast display, ribosome display, bacterial display). Display libraries, e.g., phage display libraries, are available (and/or can be generated by one of ordinary skill in the art) that can be screened to identify an antibody that binds to an antigen of interest, e.g., using panning. See, e.g., Sidhu, S. (ed.) Phage Display in Biotechnology and Drug Discovery (Drug Discovery Series; CRC Press; 1st ed., 2005; Aitken, R. (ed.) Antibody Phage Display: Methods and Protocols (Methods in Molecular Biology) Humana Press; 2nd ed., 2009.

It should be appreciated that the antigen binding protein (e.g., VHH) may be an antigen binding protein that binds to an epitope of a toxin or a pathogen. It would be apparent to the skilled artisan how to generate an antigen binding protein, such as an antibody, to an antigen of interest, such as an antigen of a toxin or an antigen of a pathogen. In some embodiments, the antigen binding protein binds to an antigen of a toxin. As used herein the term “toxin” refers to a poisonous substance produced within living cells or organisms as well as toxins created by artificial processes. Accordingly, toxins include natural and synthetic toxins. In some embodiments, the toxin is a substance produced within a living cell or organism. In some embodiments, the toxin is a substance created by an artificial process. In some embodiments, the toxin is a biotoxin. Examples of biotoxins include, without limitation cyanotoxins (e.g., produced by cyanobacteria), dinotoxins, (e.g., produced by dinoflagellates), necrotoxins (e.g., toxins from spiders, snakes or necrotizing fasciitis), neurotoxins (e.g., toxins from scorpions, black widow spiders, box jellyfish, elapid snakes, cone snails, blue-ringed octopi, venomous fish, frogs, palythoa coral, algae, cyanobacteria and dinoflagellates), myotoxins (e.g., toxins from rattlesnakes and eastern bearded dragons), and cytotoxins (e.g., ricin, apitoxin and T-2 mycotoxin). In some embodiments, the toxin selected from the group consisting of botulinum toxin, tetanus toxin, diphtheria toxin, dioxin, muscarine, bufotoxin, sarin, anthrax toxin, shiga toxin (e.g., naturally produced by Shigella dysenteriae), Shiga-like toxins 1 and 2 (e.g., naturally produced by certain strains of Escherichia coli), pertussis toxin (e.g., naturally produced by Bordetealla pertussis), one or more components of a venom (e.g., from a snake, spider, scorpion, jellyfish or wasp), and ricin. It should be appreciated, however, that antigen binding proteins may be designed to bind other toxins and are within the scope of this disclosure.

In some embodiments, the antigen binding protein (e.g., VHH) may be an antigen binding protein that binds to an epitope of botulinum toxin (e.g., botuninum toxin types A, B, C, D, E, F or G). Botulinum toxin (abbreviated as BTX or BoNT) is produced by Clostridium botulinum, a gram-positive anaerobic bacterium. The clinical syndrome of botulism can occur following ingestion of contaminated food, from colonization of the gastrointestinal tract, or from a wound infection. Typically, BoNT is broken into 7 neurotoxins (labeled as types A, B, C [C1, C2], D, E, F, and G), which are antigenically and serologically distinct but structurally similar. Human botulism is caused mainly by types A, B, E, and in certain cases, F. Types C and D can cause toxicity in animals other than humans. The BoNT molecule is synthesized as a single chain (150 kD) and then cleaved to form the dichain molecule with a disulfide bridge. The light chain (˜50 kD) acts as a zinc (Zn2+) endopeptidase similar to tetanus toxin with proteolytic activity located at the N-terminal end. The heavy chain (˜100 kD) provides cholinergic specificity and is responsible for binding the toxin to presynaptic receptors; it also promotes light-chain translocation across the endosomal membrane. Botulinum toxins (e.g., botulinum toxin types A, B, C, D, E, F and G) are known in the art and have been described previously, for example, in Henkel J. S., et al., “Toxins from Bacteria”. EXS, 2010; 100: 1-29; and Shukla H. D., “Clostridium botulinum: a bug with beauty and weapon”. Critical Reviews in Microbiology, 2005, 31 (1): 11-18.; the contents of each of which are incorporated herein by reference.

It should be appreciated that the antigen binding protein (e.g., VHH) may be an antigen binding protein that binds to an epitope of a pathogen. It would be apparent to the skilled artisan how to generate an antigen binding protein, such as an antibody, to an antigen of interest, such as an antigen of a pathogen. In some embodiments, the antigen binding protein binds to an antigen of a pathogen. A “pathogen” refers to an agent that can produce disease. Typically, a pathogen refers to an infectious agent such as a virus, bacterium, protozoa, prion, fungus, or other micro-organism. As used herein an “antigen of a pathogen” refers to a portion of a pathogen that is capable of eliciting an immune response. In some embodiments, the antigen of a pathogen is a protein, which may be produced by a pathogenic organism. In some embodiments, the antigen of a pathogen is a cell surface protein that is produced by the pathogenic organism (e.g., virus, bacterium, protozoa, or fungus).

In some embodiments, the antigen of a pathogen is from a virus. Exemplary viruses include, e.g., Retroviridae (e.g., lentiviruses such as human immunodeficiency viruses, such as HIV-I); Caliciviridae (e.g. strains that cause gastroenteritis); Togaviridae (e.g., equine encephalitis viruses, rubella viruses); Flaviridae (e.g. dengue viruses, encephalitis viruses, yellow fever viruses, hepatitis C virus); Coronaviridae (e.g. coronaviruses); Rhabdoviridae (e.g. vesicular stomatitis viruses, rabies viruses); Filoviridae (e.g. Ebola viruses); Paramyxoviridae (e.g. parainfluenza viruses, mumps virus, measles virus, respiratory syncytial virus); Orthomyxoviridae (e.g. influenza viruses); Bunyaviridae (e.g. Hantaan viruses, bunga viruses, phleboviruses and Nairo viruses); Arenaviridae (hemorrhagic fever viruses); Reoviridae (erg., reoviruses, orbiviurses and rotaviruses); Birnaviridae; Hepadnaviridae (Hepatitis B virus); Parvoviridae (parvoviruses); Papovaviridae (papilloma viruses, polyoma viruses); Adenoviridae; Herpesviridae (herpes simplex virus (HSV) 1 and 2, varicella zoster virus, cytomegalovirus (CMV), EBV, KSV); Poxviridae (variola viruses, vaccinia viruses, pox viruses); and Picornaviridae (e.g. polio viruses, hepatitis A virus; enteroviruses, human coxsackie viruses, rhinoviruses, echoviruses). In some embodiments, the antigen of a pathogen is a viral protein (e.g., viral surface glycoprotein).

In some embodiments, the antigen of a pathogen is from a bacterium. Exemplary bacteria include, e.g., Helicobacter pylori, Borellia burgdorferi, Legionella pneumophilia, Mycobacteria (e.g., M. tuberculosis, M. avium, M. intracellulare, M. kansasii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, Campylobacter sp., Enterococcus sp., Chlamydia sp., Haemophilus influenzae, Bacillus anthracis, Corynebacterium diphtheriae, Erysipelothrix rhusiopathiae, Clostridium perfringens, Clostridium tetani, Enterobacter aerogenes, Klebsiella pneumoniae, Pasteurella multocida, Bacteroides sp., Fusobacterium nucleatum, Streptobacillus moniliformis, Treponema pallidum, Treponema pertenue, Leptospira, Actinomyces israelii and Francisella tularensis. In some embodiments, the antigen of a pathogen is a bacterial protein (e.g., bacterial cell surface protein).

In some embodiments, the antigen of a pathogen is from a fungus. Exemplary fungi include, e.g., Aspergillus, such as Aspergillus flavus, Aspergillus fumigatus, Aspergillus niger, Blastomyces, such as Blastomyces dermatitidis, Candida, such as Candida albicans, Candida glabrata, Candida guilliermondii, Candida krusei, Candida parapsilosis, Candida tropicalis, Coccidioides, such as Coccidioides immitis, Cryptococcus, such as Cryptococcus neoformans, Epidermophyton, Fusarium, Histoplasma, such as Histoplasma capsulatum, Malassezia, such as Malassezia furfur, Microsporum, Mucor, Paracoccidioides, such as Paracoccidioides brasiliensis, Penicillium, such as Penicillium marneffei, Pichia, such as Pichia anomala, Pichia guilliermondii, Pneumocystis, such as Pneumocystis carinii, Pseudallescheria, such as Pseudallescheria boydii, Rhizopus, such as Rhizopus oryzae, Rhodotorula, such as Rhodotorula rubra, Scedosporium, such as Scedosporium apiospermum, Schizophyllum, such as Schizophyllum commune, Sporothrix, such as Sporothrix schenckii, Trichophyton, such as Trichophyton mentagrophytes, Trichophyton rubrum, Trichophyton verrucosum, Trichophyton violaceutn, Trichosporon, such as Trichosporon asahii, Trichosporon cutaneum, Trichosporon inkin, and Trichosporon mucoides. In some embodiments, the antigen of a pathogen is a fungal protein (e.g., bacterial cell surface protein).

In some embodiments, the pathogen is a tumor cell. In some embodiments, an antigen is a tumor antigen (TA). In general, a tumor antigen can be any antigenic substance produced by tumor cells (e.g., tumorigenic cells or in some embodiments tumor stromal cells, e.g., tumor-associated cells such as cancer-associated fibroblasts). In some embodiments, a tumor antigen is a molecule (or portion thereof) that is differentially expressed by tumor cells as compared with non-tumor cells. Tumor antigens may include, e.g., proteins that are normally produced in very small quantities and are expressed in larger quantities by tumor cells, proteins that are normally produced only in certain stages of development, proteins whose structure (e.g., sequence or post-translational modification(s)) is modified due to mutation in tumor cells, or normal proteins that are (under normal conditions) sequestered from the immune system. Tumor antigens may be useful in, e.g., identifying or detecting tumor cells (e.g., for purposes of diagnosis and/or for purposes of monitoring subjects who have received treatment for a tumor, e.g., to test for recurrence) and/or for purposes of targeting various agents (e.g., therapeutic agents) to tumor cells. In some embodiments, a TA is an expression product of a mutated gene, e.g., an oncogene or mutated tumor suppressor gene, an overexpressed or aberrantly expressed cellular protein, an antigen encoded by an oncogenic virus (e.g., HBV; HCV; herpesvirus family members such as EBV, KSV; papilloma virus, etc.), or an oncofetal antigen. Oncofetal antigens are normally produced in the early stages of embryonic development and largely or completely disappear by the time the immune system is fully developed. Examples are alphafetoprotein (AFP, found, e.g., in germ cell tumors and hepatocellular carcinoma) and carcinoembryonic antigen (CEA, found, e.g., in bowel cancers and occasionally lung or breast cancer). Tyrosinase is an example of a protein normally produced in very low quantities but whose production is greatly increased in certain tumor cells (e.g., melanoma cells). Other exemplary TAs include, e.g., CA-125 (found, e.g., in ovarian cancer); MUC-1 (found, e.g., in breast cancer); epithelial tumor antigen (found, e.g., in breast cancer); melanoma-associated antigen (MAGE; found, e.g., in malignant melanoma); prostatic acid phosphatase (PAP, found in prostate cancer). In some embodiments, a TA is at least in part exposed at the cell surface of tumor cells. In some embodiments, a tumor antigen comprises an abnormally modified polypeptide or lipid, e.g., an aberrantly modified cell surface glycolipid or glycoprotein. It will be appreciated that a TA may be expressed by a subset of tumors of a particular type and/or by a subset of cells in a tumor.

In some embodiments, any of the fusion proteins provided herein comprise two or more (e.g., two, three, four, five, or more) antigen binding proteins. In some embodiments, the fusion protein further comprises a second antigen binding protein. In some embodiments, the fusion protein comprises a first antigen binding protein that binds to a first antigen, and a second antigen that binds to a second antigen. In some embodiments, the first antigen and the second antigen are the same. In some embodiments, the first antigen and the second antigen are different. In some embodiments, the first antigen is a first epitope of a toxin and the second antigen is a second epitope of the same toxin. In some embodiments, the first antigen is a first epitope of an antigen of a pathogen and the second antigen is a second epitope of the same antigen of the pathogen.

In some embodiments, any of the fusion proteins provided herein comprise one or more VHH antigen binding proteins. In some embodiments, the fusion protein comprises one, two, three, four, five, or more VHHs. In some embodiments, the fusion protein comprises one VHH. In some embodiments, the fusion protein comprises two VHHs. In some embodiments, the fusion protein comprises a VHH having an amino acid sequence that is at least 70%, at least 75%, at least 85%, at least 90%, at least 91%, at least 92%, at least 93%, at least 94%, at least 95%, at least 96%, at least 97%, at least 98%, at least 99%, or at least 99.5% identical to the amino acid sequence of any one of SEQ ID NOs: 15-18. In some embodiments, the fusion protein comprises a VHH having the amino acid sequence of any one of SEQ ID NOs: 15-18. In some embodiments, the fusion comprises two VHHs. In some embodiments, the fusion protein comprises two VHHs that are at least 70%, at least 75%, at least 85%, at least 90%, at least 91%, at least 92%, at least 93%, at least 94%, at least 95%, at least 96%, at least 97%, at least 98%, at least 99%, or at least 99.5% identical to the the amino acid sequences of SEQ ID NOs: 15 and 16. In some embodiments, the fusion protein comprises two VHHs that comprise the amino acid sequences of SEQ ID NOs: 15 and 16. In some embodiments, the fusion protein comprises two VHHs that are at least 70%, at least 75%, at least 85%, at least 90%, at least 91%, at least 92%, at least 93%, at least 94%, at least 95%, at least 96%, at least 97%, at least 98%, at least 99%, or at least 99.5% identical to the amino acid sequences of SEQ ID NOs: 17 and 18. In some embodiments, the fusion protein comprises two VHHs that comprise the amino acid sequences of SEQ ID NOs: 17 and 18. In some embodiments, the fusion protein comprises an amino acid sequence that is at least 70%, at least 75%, at least 85%, at least 90%, at least 91%, at least 92%, at least 93%, at least 94%, at least 95%, at least 96%, at least 97%, at least 98%, at least 99%, or at least 99.5% identical to the amino acid sequence of SEQ ID NO: 13 or SEQ ID NO: 14. In some embodiments, the fusion protein comprises the amino acid sequence of SEQ ID NO: 13 or SEQ ID NO: 14.

VHH1: H7:
(SEQ ID NO: 15)
SGGGLVQVGGSLRLSCVVSGSDISGIAMGWYRQAPGKRREMVADIFS
GGSTDYAGSVKGRFTISRDNAKKTSYLQMNNVKPEDTGVYYCRLYGS
GDYWGQGTQVTVSS
VHH2: B5
(SEQ ID NO: 16)
SGGGLVHPGGSLRLSCAPSASLPSTPFNPFNNMVGWYRQAPGKQREM
VASIGLRINYADSVKGRFTISRDNAKNTVDLQMDSLRPEDSATYYCH
IEYTHYWGKGTLVTVSS
VHH1: D10
(SEQ ID NO: 17)
SGGGLVQPGGSLRLSCAASGFTLDSYAIGWFRQAPGKEREGVACISA
SGSGTDYVDSVKGRFTVSRDQAKSMVFLQMNNMKPEDAAVYYCAADY
RPRPLPIQAPCTMTGGNYWGQGTQVTVSS
VHH2: G10
(SEQ ID NO: 18)
SGGGLVQAGGSLRLSCAASILTYDLDYYYIGWVRQAPGKEREGVSCI
SSTDGATYYADSVKGRFTISRNNAKNTVYLQMNNLKPEDTAIYYCAA
APLAGRYCPASHEYGYWGQGTQVTVSS
VNA/A:
(SEQ ID NO: 13)
QGVQAQLQLVESGGGLVQVGGSLRLSCVVSGSDISGIAMGWYRQAPG
KRREMVADIFSGGSTDYAGSVKGRFTISRDNAKKTSYLQMNNVKPED
TGVYYCRLYGSGDYWGQGTQVTVSSAHHSEDPTSAIAGGGGSGGGGS
GGGGSLQGQLQLVESGGGLVHPGGSLRLSCAPSASLPSTPFNPFNNM
VGWYRQAPGKQREMVASIGLRINYADSVKGRFTISRDNAKNTVDLQM
DSLRPEDSATYYCHIEYTHYWGKGTLVTVSSEPKTPKPQP
VNA/B:
(SEQ ID NO: 14)
QGVQAQLQLVESGGGLVQPGGSLRLSCAASGFTLDSYAIGWFRQAPG
KEREGVACISASGSGTDYVDSVKGRFTVSRDQAKSMVFLQMNNMKPE
DAAVYYCAADYRPRPLPIQAPCTMTGGNYWGQGTQVTVSSEPKTPKP
QAIAGGGGSGGGGSGGGGSLQGQLQLVESGGGLVQAGGSLRLSCAAS
ILTYDLDYYYIGWVRQAPGKEREGVSCISSTDGATYYADSVKGRFTI
SRNNAKNTVYLQMNNLKPEDTAIYYCAAAPLAGRYCPASHEYGYWGQ
GTQVTVSSAHHSEDPSS

It should be appreciated that the red blood cell transmembrane protein and an antigen binding protein (e.g., a first and/or a second antigen binding protein) of the fusion protein are fused via a linker. In some embodiments, a first antigen binding protein and a second antigen binding protein are fused via a linker. The term “linker,” as used herein, refers to a chemical group or a molecule linking two molecules or moieties, e.g., two domains of a fusion protein, such as, for example, a red blood cell transmembrane protein and an antigen binding protein. In some embodiments, a linker joins a red blood cell transmembrane protein and a first antigen binding protein. In some embodiments, a linker joins a red blood cell transmembrane protein and a second antigen binding protein. In some embodiments, a linker joins a first antigen binding protein and a second antigen binding protein. Typically, the linker is positioned between, or flanked by, two groups, molecules, or other moieties and connected to each one via a covalent bond, thus connecting the two. In some embodiments, the linker is a covalent bond. In some embodiments, the linker is an atom. In some embodiments, the linker is an amino acid or a plurality of amino acids (e.g., a peptide or protein). In some embodiments, the linker is an organic molecule, group, polymer, or chemical moiety. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated. In some embodiments, the linker comprises the amino acid sequence set forth in any one of SEQ ID NOs: 19-23. In some embodiments, any of the fusion proteins provided herein comprise one or more tags (e.g., an epitope tag). Exemplary tags include, without limitation, FLAG tags, polyhistidine (His) tags (e.g., 1, 2, 3, 4, 5, 6, 7, 8, 9, or 10 histidine residues), HA tags, Myc tags, and glutathione-S-transferase (GST) tags. Additional epitope tags are known in the art and have been described previously, for example, in Brizzard B., “Epitope tagging,” BioTechniques, Vol. 44, No. 5, April 2008, pp. 693-695; the entire contents of which are hereby incorporated by reference. In some embodiments, the epitope tag is a myc tag. In some embodiments, the myc tag comprises the amino acid sequence EQKLISEEDL (SEQ ID NO: 24).

Exemplary Linkers:

(SEQ ID NO: 19)
QGVQAQLQLVE
(SEQ ID NO: 20)
AHHSEDPTSAIAGGGGSGGGGSGGGGSLQGQLQLVE
(SEQ ID NO: 21)
EPKTPKPQP
(SEQ ID NO: 22)
EPKTPKPQAIAGGGGSGGGGSGGGGSLQGQLQLVE
(SEQ ID NO: 23)
AHHSEDPSS

In some embodiments, the fusion protein further comprises a signal peptide. A signal peptide, typically referred to as signal sequence, targeting signal, localization signal, localization sequence, transit peptide, leader sequence or leader peptide is a short peptide (e.g., from 15-30 amino acids long) capable of targeting a protein to a cell membrane. In some embodiments, the signal peptide is at the N-terminus of the fusion protein. In some embodiments, the signal peptide is at the C-terminus of the fusion protein. Signal peptides are heterogeneous and many prokaryotic and eukaryotic signal peptides are functionally interchangeable even between different species. Exemplary signal peptides have been described in the art, for example, in Kober L., et al., “Optimized signal peptides for the development of high expressing CHO cell lines,” Biotechnol. Bioeng., April 2013, 110 (4): 1164-73; and Von Heijne G., “Signal sequences: The limits of variation”. J Mol Biol., July 1985, 184 (1): 99-105; the entire contents of each of which are incorporated by reference herein. In some embodiments, the signal peptide comprises the amino acid sequence

(SEQ ID NO: 24)
MYGKIIFVLLLSEIVSISA.

In some embodiments, the fusion protein comprises any one of the following structures:

NH₂-[red blood cell transmembrane protein]-[first antigen binding protein]-COOH,
NH₂-[first antigen binding protein]-[red blood cell transmembrane protein]-COOH,
NH₂-[red blood cell transmembrane protein]-[first antigen binding protein]-[second antigen binding protein]-COOH,
NH₂-[first antigen binding protein]-[red blood cell transmembrane protein]-[second antigen binding protein]-COOH, and
NH₂-[first antigen binding protein]-[second antigen binding protein]-[red blood cell transmembrane protein]-COOH, wherein NH₂ is the N-terminus of the fusion protein, and COOH is the C-terminus of the fusion protein. In some embodiments, the “]-[” used in the general architecture above indicates the presence of an optional linker sequence. In some embodiments, the fusion protein comprises a signal sequence. In some embodiments, the fusion protein comprises a signal sequence at the N-terminus of the fusion protein. In some embodiments, the fusion protein comprises a signal sequence at the C-terminus of the fusion protein. In some embodiments, the fusion protein comprises an epitope tag. In some embodiments, the fusion protein comprises an epitope tag at the N-terminus of the fusion protein. In some embodiments, the fusion protein comprises an epitope tag at the C-terminus of the fusion protein. In some embodiments, the fusion protein comprises an epitope tag between the signal peptide and a first or second antigen binding protein of the fusion protein. In some embodiments, the fusion protein comprises an epitope tag between the first antigen binding protein and the second antigen binding protein.

In some embodiments, the fusion protein comprises the structure NH₂-[first VHH]-[glycophorin A]-COOH; NH₂-[second VHH]-[first VHH]-[glycophorin A]-COOH; NH₂-[Kell]-[first VHH]-COOH; or NH₂-[Kell]-[first VHH]-[second VHH]-COOH. In some embodiments, the fusion protein comprises an amino acid sequence that is at least 70%, at least 75%, at least 85%, at least 90%, at least 91%, at least 92%, at least 93%, at least 94%, at least 95%, at least 96%, at least 97%, at least 98%, at least 99%, or at least 99.5% identical to the amino acid sequence of any one of SEQ ID NOs: 2, 4, 6, or 8. In some embodiments, the fusion protein comprises the amino acid sequence of any one of SEQ ID NOs: 2, 4, 6, or 8. In some embodiments, the fusion protein is encoded by any one of the nucleic acid sequences of SEQ ID NO: 1, 3, 5, or 7.

### Fusion Protein Sequences:

Nucleic Acid Sequence Encoding GPA-VNA/A:

(SEQ ID NO: 1)
ATGTATGGAAAAATAATCTTTGTATTACTATTGTCAGAAATTGTGAG
CATATCAGCAGAACAGAAACTGATCTCTGAAGAAGACCTGCAAGGTG
TTCAAGCTCAACTGCAGCTCGTGGAGTCAGGTGGAGGCTTGGTGCAG
GTTGGGGGGTCTCTGAGACTCTCCTGTGTAGTTTCTGGAAGCGACAT
CAGTGGCATTGCGATGGGCTGGTACCGCCAGGCTCCAGGGAAGCGGC
GCGAAATGGTCGCAGATATTTTTTCTGGCGGTAGTACAGACTATGCA
GGCTCCGTGAAGGGCCGATTCACCATCTCCAGAGACAACGCCAAGAA
GACGAGCTATCTGCAAATGAACAACGTGAAACCTGAGGACACCGGAG
TCTACTACTGTAGGCTGTACGGGAGCGGTGACTACTGGGGCCAGGGG
ACCCAGGTCACCGTCTCCTCAGCGCACCACAGCGAAGACCCCACTAG
TGCGATCGCTGGTGGAGGCGGTTCAGGCGGAGGTGGCTCTGGCGGTG
GCGGTTCCCTGCAGGGTCAGTTGCAGCTCGTGGAGTCCGGCGGAGGC
TTGGTGCACCCTGGGGGGTCTCTGAGACTCTCTTGTGCACCCTCTGC
CAGTCTACCATCAACACCCTTCAACCCCTTCAACAATATGGTGGGCT
GGTACCGTCAGGCTCCAGGTAAACAGCGCGAAATGGTCGCAAGTATT
GGTCTACGAATAAACTATGCAGACTCCGTGAAGGGCCGATTCACCAT
CTCCAGAGACAACGCCAAGAACACGGTGGATCTGCAGATGGACAGCC
TGCGACCTGAGGACTCAGCCACATACTACTGTCATATAGAATACACC
CACTACTGGGGCAAAGGGACCCTGGTCACCGTCTCCTCGGAACCCAA
GACACCAAAACCACAACCGTTAAGTACCACTGAGGTGGCAATGCACA
CTTCAACTTCTTCTTCAGTCACAAAGAGTTACATCTCATCACAGACA
AATGATACGCACAAACGGGACACATATGCAGCCACTCCTAGAGCTCA
TGAAGTTTCAGAAATTTCTGTTAGAACTGTTTACCCTCCAGAAGAGG
AAACCGGAGAAAGGGTACAACTTGCCCATCATTTCTCTGAACCAGAG
ATAACACTCATTATTTTTGGGGTGATGGCTGGTGTTATTGGAACGAT
CCTCTTAATTTCTTACGGTATTCGCCGACTGATAAAGAAAAGCCCAT
CTGATGTAAAACCTCTCCCCTCACCTGACACAGACGTGCCTTTAAGT
TCTGTTGAAATAGAAAATCCAGAGACAAGTGATCAATGA

Amino acid sequence of GPA-VNA/A, where VHH-H7 is indicated in bold, VHH-B5 is indicated in bold and underlining, GPA is indicated in italics, the GPA signal peptide is indicated in italics and underlining, and the Myc tag is indicated in underlining:

(SEQ ID NO: 2)

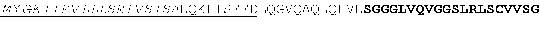

SDISGIAMGWYRQAPGKRREMVADIFSGGSTDYAGSVKGRFTISRDNAKKTSYLQMNNVK
PEDTGVYYCRLYGSGDYWGQGTQVTVSSAHHSEDPTSAIAGGGGSGGGGSGGGGSLQGQL

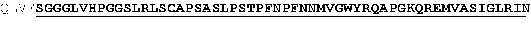

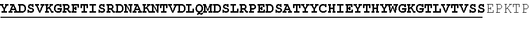

KPQPLSTTEVAMHTSTSSSVTKSYISSQTNDTHKRDTYAATPRAHEVSEISVRTVYPPEE
ETGERVQLAHHFSEPEITLIIFGVMAGVIGTILLISYGIRRLIKKSPSDVKPLPSPDTDV
PLSSVEIENPETSDQ

Nucleic Acid Sequence Encoding GPA-VNA/B:

(SEQ ID NO: 3)
ATGTATGGAAAAATAATCTTTGTATTACTATTGTCAGAAATTGTGAG
CATATCAGCAGAACAGAAACTGATCTCTGAAGAAGACCTGCAAGGTG
TTCAAGCTCAACTGCAGCTCGTGGAGTCAGGGGGAGGCTTGGTGCAG
CCTGGGGGGTCTCTGAGACTCTCCTGTGCAGCCTCTGGATTCACTTT
AGATAGTTATGCAATAGGCTGGTTCCGCCAGGCCCCAGGGAAGGAGC
GTGAGGGGGTCGCATGTATTAGTGCTAGTGGTAGTGGCACGGACTAT
GTAGACTCCGTGAAGGGCCGATTCACCGTCTCCAGAGACCAGGCCAA
GAGCATGGTGTTTCTGCAAATGAACAACATGAAACCTGAGGACGCAG
CCGTTTATTACTGTGCAGCAGATTATCGGCCGAGGCCCCTGCCGATT
CAGGCGCCGTGTACAATGACAGGTGGCAACTACTGGGGCCAGGGGAC
CCAGGTCACCGTCTCCTCAGAACCCAAGACACCAAAACCACAAGCGA
TCGCTGGTGGAGGCGGTTCAGGCGGAGGTGGCTCTGGCGGTGGCGGT
TCCCTGCAGGGTCAGTTGCAGCTCGTGGAGTCCGGTGGAGGCTTGGT
GCAGGCTGGGGGGTCTCTGAGACTCTCCTGTGCAGCCTCTATACTCA
CTTATGATTTGGATTATTATTACATAGGCTGGGTCCGCCAGGCCCCA
GGGAAGGAGCGTGAGGGGGTCTCATGTATTAGTAGTACTGATGGTGC
CACATACTATGCAGACTCCGTGAAGGGCCGATTCACCATCTCCAGAA
ACAACGCCAAGAACACGGTGTATCTGCAAATGAACAACCTAAAACCT
GAGGACACAGCCATTTATTATTGTGCAGCAGCCCCCCTGGCTGGGCG
CTACTGTCCCGCCTCGCATGAGTATGGCTACTGGGGTCAGGGGACCC
AGGTCACCGTCTCGTCAGCGCACCACAGCGAAGACCCCTCGTCCTTA
AGTACCACTGAGGTGGCAATGCACACTTCAACTTCTTCTTCAGTCAC
AAAGAGTTACATCTCATCACAGACAAATGATACGCACAAACGGGACA
CATATGCAGCCACTCCTAGAGCTCATGAAGTTTCAGAAATTTCTGTT
AGAACTGTTTACCCTCCAGAAGAGGAAACCGGAGAAAGGGTACAACT
TGCCCATCATTTCTCTGAACCAGAGATAACACTCATTATTTTTGGGG
TGATGGCTGGTGTTATTGGAACGATCCTCTTAATTTCTTACGGTATT
CGCCGACTGATAAAGAAAAGCCCATCTGATGTAAAACCTCTCCCCTC
ACCTGACACAGACGTGCCTTTAAGTTCTGTTGAAATAGAAAATCCAG
AGACAAGTGATCAATGA

Amino acid sequence of GPA-VNA/B, where VHH-D10 is indicated in bold, VHH-G10 is indicated in bold and underlining, GPA is indicated in italics, the GPA signal peptide is indicated in italics and underlining, and the Myc tag is indicated in underlining:

(SEQ ID NO: 4)

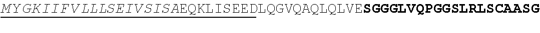

FTLDSYAIGWFRQAPGKEREGVACISASGSGTDYVDSVKGRFTVSRDQAKSMVFLQMNNM
KPEDAAVYYCAADYRPRPLPIQAPCTMTGGNYWGQGTQVTVSSEPKTPKPQAIAGGGGSG

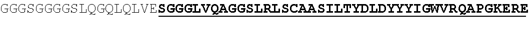

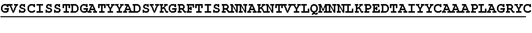

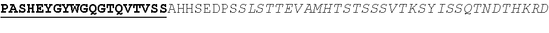

TYAATPRAHEVSEISVRTVYPPEEETGERVQLAHHFSEPEITLIIFGVMAGVIGTILLIS
YGIRRLIKKSPSDVKPLPSPDTDVPLSSVEIENPETSDQ

Nucleic Acid Sequence of Kell-VNA/A:

(SEQ ID NO: 5)
ATGGAAGGTGGGGACCAAAGTGAGGAAGAGCCGAGGGAACGCAGCCAGGCAGGTGGAATG
GGAACTCTCTGGAGCCAAGAGAGCACTCCAGAAGAGAGGCTGCCCGT
GGAAGGGAGCAGGCCATGGGCAGTGGCCAGGCGGGTGCTGACAGCTATCCTGAT
TTTGGGCCTGCTCCTTTGTTTTTCTGTGCTTTTGTTCTACAACTTCCAGAACTGTGG
CCCTCGCCCCTGTGAGACATCTGTGTGTTTGGATCTCCGGGATCATTACCTGGCCT
CTGGGAACACAAGTGTGGCCCCCTGCACCGACTTCTTCAGCTTTGCCTGTGGAAG
GGCCAAAGAGACCAATAATTCTTTTCAGGAGCTTGCCACAAAGAACAAAAACCG
ACTTCGGAGAATACTGGAGGTCCAGAATTCCTGGCACCCAGGCTCTGGGGAGGA
GAAAGCCTTCCAGTTCTACAACTCCTGCATGGATACACTTGCCATTGAAGCTGCA
GGGACTGGTCCCCTCAGACAAGTTATTGAGGAGCTTGGAGGCTGGCGCATCTCTG
GTAAATGGACTTCCTTAAACTTTAACCGAACGCTGAGACTTCTGATGAGTCAGTA
TGGCCATTTCCCTTTCTTCAGAGCCTACCTAGGACCTCATCCTGCCTCTCCACACA
CACCAGTCATCCAGATAGACCAGCCAGAGTTTGATGTTCCCCTCAAGCAAGATCA
AGAACAGAAGATCTATGCCCAGATCTTTCGGGAATACCTGACTTACCTGAATCAG
CTGGGAACCTTGCTGGGAGGAGACCCAAGCAAGGTGCAAGAACACTCTTCCTTG
TCAATCTCCATCACTTCACGGCTGTTCCAGTTTCTGAGGCCCCTGGAGCAGCGGC
GGGCACAGGGCAAGCTCTTCCAGATGGTCACTATCGACCAGCTCAAGGAAATGG
CCCCCGCCATCGACTGGTTGTCCTGCTTGCAAGCGACATTCACACCGATGTCCCT
GAGCCCTTCTCAGTCCCTCGTGGTCCATGACGTGGAATATTTGAAAAACATGTCA
CAACTGGTGGAGGAGATGCTGCTAAAGCAGAGGGACTTTCTGCAGAGCCACATG
ATCTTAGGGCTGGTGGTGACCCTTTCTCCAGCCCTGGACAGTCAATTCCAGGAGG
CACGCAGAAAGCTCAGCCAGAAACTGCGGGAACTGACAGAGCAACCACCCATGC
CTGCCCGCCCACGATGGATGAAGTGCGTGGAGGAGACAGGCACGTTCTTCGAGC
CCACGCTGGCGGCTTTGTTTGTTCGTGAGGCCTTTGGCCCGAGCACCCGAAGTGC
TGCCATGAAATTATTCACTGCGATCCGGGATGCCCTCATCACTCGCCTCAGAAAC
CTTCCCTGGATGAATGAGGAGACCCAGAACATGGCCCAGGACAAGGTTGCTCAA
CTGCAGGTGGAGATGGGGGCTTCAGAATGGGCCCTGAAGCCAGAGCTGGCCCGA
CAAGAATACAACGATATACAGCTTGGATCGAGCTTCCTGCAGTCTGTCCTGAGCT
GTGTCCGGTCCCTCCGAGCTAGAATTGTCCAGAGCTTCTTGCAGCCTCACCCCCA
ACACAGGTGGAAGGTGTCCCCTTGGGACGTCAATGCTTACTATTCGGTATCTGAC
CATGTGGTAGTCTTTCCAGCTGGACTCCTCCAACCCCCATTCTTCCACCCTGGCTA
TCCCAGAGCCGTGAACTTTGGCGCTGCTGGCAGCATCATGGCCCACGAGCTGTTG
CACATCTTCTACCAGCTCTTACTGCCTGGGGGCTGCCTCGCCTGTGACAACCATG
CCCTCCAGGAAGCTCACCTGTGCCTGAAGCGCCATTATGCTGCCTTTCCATTACC
TAGCAGAACCTCCTTCAATGACTCCCTCACATTCTTAGAGAATGCTGCAGACGTT
GGGGGGCTAGCCATCGCGCTGCAGGCATACAGCAAGAGGCTGTTACGGCACCAT
GGGGAGACTGTCCTGCCCAGCCTGGACCTCAGCCCCCAGCAGATCTTCTTTCGAA
GCTATGCCCAGGTGATGTGTAGGAAGCCCAGCCCCCAGGACTCTCACGACACTC
ACAGCCCTCCACACCTCCGAGTCCACGGGCCCCTCAGCAGCACCCCAGCCTTTGC
CAGGTATTTCCGCTGTGCACGTGGTGCTCTCTTGAACCCCTCCAGCCGCTGCCAG
CTCTGGCAAGGTGTTCAAGCTCAACTGCAGCTCGTGGAGTCAGGTGGAGGCTTGG
TGCAGGTTGGGGGGTCTCTGAGACTCTCCTGTGTAGTTTCTGGAAGCGACATCAG
TGGCATTGCGATGGGCTGGTACCGCCAGGCTCCAGGGAAGCGGCGCGAAATGGT
CGCAGATATTTTTTCTGGCGGTAGTACAGACTATGCAGGCTCCGTGAAGGGCCGA
TTCACCATCTCCAGAGACAACGCCAAGAAGACGAGCTATCTGCAAATGAACAAC
GTGAAACCTGAGGACACCGGAGTCTACTACTGTAGGCTGTACGGGAGCGGTGAC
TACTGGGGCCAGGGGACCCAGGTCACCGTCTCCTCAGCGCACCACAGCGAAGAC
CCCACTAGTGCGATCGCTGGTGGAGGCGGTTCAGGCGGAGGTGGCTCTGGCGGT
GGCGGTTCCCTGCAGGGTCAGTTGCAGCTCGTGGAGTCCGGCGGAGGCTTGGTGC
ACCCTGGGGGGTCTCTGAGACTCTCTTGTGCACCCTCTGCCAGTCTACCATCAAC
ACCCTTCAACCCCTTCAACAATATGGTGGGCTGGTACCGTCAGGCTCCAGGTAAA
CAGCGCGAAATGGTCGCAAGTATTGGTCTACGAATAAACTATGCAGACTCCGTG
AAGGGCCGATTCACCATCTCCAGAGACAACGCCAAGAACACGGTGGATCTGCAG
ATGGACAGCCTGCGACCTGAGGACTCAGCCACATACTACTGTCATATAGAATAC
ACCCACTACTGGGGCAAAGGGACCCTGGTCACCGTCTCCTCGGAACCCAAGACA
CCAAAACCACAACCGGAACAGAAACTGATCTCTGAAGAAGACCTGTAA

Kell-VNA/A: Amino acid sequence of Kell-VNA/A, where VHH-H7 is indicated in bold, VHH-B5 is indicated in bold and underlining, GPA is indicated in italics, and the Myc tag is indicated in underlining:

(SEQ ID NO: 6)
MEGGDQSEEEPRERSQAGGMGTLWSQESTPEERLPVEGSRPWAVARRVLTAILILGLLLC
FSVLLFYNFQNCGPRPCETSVCLDLRDHYLASGNTSVAPCTDFFSFACGRAKETNNSFQEL
ATKNKNRLRRILEVQNSWHPGSGEEKAFQFYNSCMDTLAIEAAGTGPLRQVIEELGGWRIS
GKWTSLNFNRTLRLLMSQYGHFPFFRAYLGPHPASPHTPVIQIDQPEFDVPLKQDQEQKI
YAQIFREYLTYLNQLGTLLGGDPSKVQEHSSLSISITSRLFQFLRPLEQRRAQGKLFQMVT
IDQLKEMAPAIDWLSCLQATFTPMSLSPSQSLVVHDVEYLKNMSQLVEEMLLKQRDFLQS
HMILGLVVTLSPALDSQFQEARRKLSQKLRELTEQPPMPARPRWMKCVEETGTFFEPTLA
ALFVREAFGPSTRSAAMKLFTAIRDALITRLRNLPWMNEETQNMAQDKVAQLQVEMGASE
WALKPELARQEYNDIQLGSSFLQSVLSCVRSLRARIVQSFLQPHPQHRWKVSPWDVNAYYS
VSDHVVVFPAGLLQPPFFHPGYPRAVNFGAAGSIMAHELLHIFYQLLLPGGCLACDNHAL
QEAHLCLKRHYAAFPLPSRTSFNDSLTFLENAADVGGLAIALQAYSKRLLRHHGETVLPSL
DLSPQQIFFRSYAQVMCRKPSPQDSHDTHSPPHLRVHGPLSSTPAFARYFRCARGALLNPS
SRCQLWQGVQAQLQLVESGGGLVQVGGSLRLSCVVSGSDISGIAMGWYRQAPGKRREMVAD
IFSGGSTDYAGSVKGRFTISRDNAKKTSYLQMNNVKPEDTGVYYCRLYGSGDYWGQGTQVT

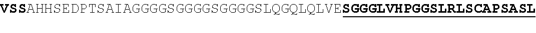

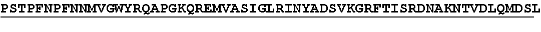

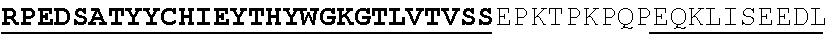

Nucleic Acid Sequence of Kell-VNA/B:

(SEQ ID NO: 7)
ATGGAAGGTGGGGACCAAAGTGAGGAAGAGCCGAGGGAACGCAGCCAGGCAGGTGGAATG
GGAACTCTCTGGAGCCAAGAGAGCACTCCAGAAGAGAGGCTGCCCGT
GGAAGGGAGCAGGCCATGGGCAGTGGCCAGGCGGGTGCTGACAGCTATCCTGAT
TTTGGGCCTGCTCCTTTGTTTTTCTGTGCTTTTGTTCTACAACTTCCAGAACTGTGG
CCCTCGCCCCTGTGAGACATCTGTGTGTTTGGATCTCCGGGATCATTACCTGGCCT
CTGGGAACACAAGTGTGGCCCCCTGCACCGACTTCTTCAGCTTTGCCTGTGGAAG
GGCCAAAGAGACCAATAATTCTTTTCAGGAGCTTGCCACAAAGAACAAAAACCG
ACTTCGGAGAATACTGGAGGTCCAGAATTCCTGGCACCCAGGCTCTGGGGAGGA
GAAAGCCTTCCAGTTCTACAACTCCTGCATGGATACACTTGCCATTGAAGCTGCA
GGGACTGGTCCCCTCAGACAAGTTATTGAGGAGCTTGGAGGCTGGCGCATCTCTG
GTAAATGGACTTCCTTAAACTTTAACCGAACGCTGAGACTTCTGATGAGTCAGTA
TGGCCATTTCCCTTTCTTCAGAGCCTACCTAGGACCTCATCCTGCCTCTCCACACA
CACCAGTCATCCAGATAGACCAGCCAGAGTTTGATGTTCCCCTCAAGCAAGATCA
AGAACAGAAGATCTATGCCCAGATCTTTCGGGAATACCTGACTTACCTGAATCAG
CTGGGAACCTTGCTGGGAGGAGACCCAAGCAAGGTGCAAGAACACTCTTCCTTG
TCAATCTCCATCACTTCACGGCTGTTCCAGTTTCTGAGGCCCCTGGAGCAGCGGC
GGGCACAGGGCAAGCTCTTCCAGATGGTCACTATCGACCAGCTCAAGGAAATGG
CCCCCGCCATCGACTGGTTGTCCTGCTTGCAAGCGACATTCACACCGATGTCCCT
GAGCCCTTCTCAGTCCCTCGTGGTCCATGACGTGGAATATTTGAAAAACATGTCA
CAACTGGTGGAGGAGATGCTGCTAAAGCAGAGGGACTTTCTGCAGAGCCACATG
ATCTTAGGGCTGGTGGTGACCCTTTCTCCAGCCCTGGACAGTCAATTCCAGGAGG
CACGCAGAAAGCTCAGCCAGAAACTGCGGGAACTGACAGAGCAACCACCCATGC
CTGCCCGCCCACGATGGATGAAGTGCGTGGAGGAGACAGGCACGTTCTTCGAGC
CCACGCTGGCGGCTTTGTTTGTTCGTGAGGCCTTTGGCCCGAGCACCCGAAGTGC
TGCCATGAAATTATTCACTGCGATCCGGGATGCCCTCATCACTCGCCTCAGAAAC
CTTCCCTGGATGAATGAGGAGACCCAGAACATGGCCCAGGACAAGGTTGCTCAA
CTGCAGGTGGAGATGGGGGCTTCAGAATGGGCCCTGAAGCCAGAGCTGGCCCGA
CAAGAATACAACGATATACAGCTTGGATCGAGCTTCCTGCAGTCTGTCCTGAGCT
GTGTCCGGTCCCTCCGAGCTAGAATTGTCCAGAGCTTCTTGCAGCCTCACCCCCA
ACACAGGTGGAAGGTGTCCCCTTGGGACGTCAATGCTTACTATTCGGTATCTGAC
CATGTGGTAGTCTTTCCAGCTGGACTCCTCCAACCCCCATTCTTCCACCCTGGCTA
TCCCAGAGCCGTGAACTTTGGCGCTGCTGGCAGCATCATGGCCCACGAGCTGTTG
CACATCTTCTACCAGCTCTTACTGCCTGGGGGCTGCCTCGCCTGTGACAACCATG
CCCTCCAGGAAGCTCACCTGTGCCTGAAGCGCCATTATGCTGCCTTTCCATTACC
TAGCAGAACCTCCTTCAATGACTCCCTCACATTCTTAGAGAATGCTGCAGACGTT
GGGGGGCTAGCCATCGCGCTGCAGGCATACAGCAAGAGGCTGTTACGGCACCAT
GGGGAGACTGTCCTGCCCAGCCTGGACCTCAGCCCCCAGCAGATCTTCTTTCGAA
GCTATGCCCAGGTGATGTGTAGGAAGCCCAGCCCCCAGGACTCTCACGACACTC
ACAGCCCTCCACACCTCCGAGTCCACGGGCCCCTCAGCAGCACCCCAGCCTTTGC
CAGGTATTTCCGCTGTGCACGTGGTGCTCTCTTGAACCCCTCCAGCCGCTGCCAG
CTCTGGCAAGGTGTTCAAGCTCAACTGCAGCTCGTGGAGTCAGGGGGAGGCTTG
GTGCAGCCTGGGGGGTCTCTGAGACTCTCCTGTGCAGCCTCTGGATTCACTTTAG
ATAGTTATGCAATAGGCTGGTTCCGCCAGGCCCCAGGGAAGGAGCGTGAGGGGG
TCGCATGTATTAGTGCTAGTGGTAGTGGCACGGACTATGTAGACTCCGTGAAGGG
CCGATTCACCGTCTCCAGAGACCAGGCCAAGAGCATGGTGTTTCTGCAAATGAAC
AACATGAAACCTGAGGACGCAGCCGTTTATTACTGTGCAGCAGATTATCGGCCG
AGGCCCCTGCCGATTCAGGCGCCGTGTACAATGACAGGTGGCAACTACTGGGGC
CAGGGGACCCAGGTCACCGTCTCCTCAGAACCCAAGACACCAAAACCACAAGCG
ATCGCTGGTGGAGGCGGTTCAGGCGGAGGTGGCTCTGGCGGTGGCGGTTCCCTG
CAGGGTCAGTTGCAGCTCGTGGAGTCCGGTGGAGGCTTGGTGCAGGCTGGGGGG
TCTCTGAGACTCTCCTGTGCAGCCTCTATACTCACTTATGATTTGGATTATTATTA
CATAGGCTGGGTCCGCCAGGCCCCAGGGAAGGAGCGTGAGGGGGTCTCATGTAT
TAGTAGTACTGATGGTGCCACATACTATGCAGACTCCGTGAAGGGCCGATTCACC
ATCTCCAGAAACAACGCCAAGAACACGGTGTATCTGCAAATGAACAACCTAAAA
CCTGAGGACACAGCCATTTATTATTGTGCAGCAGCCCCCCTGGCTGGGCGCTACT
GTCCCGCCTCGCATGAGTATGGCTACTGGGGTCAGGGGACCCAGGTCACCGTCTC
GTCAGCGCACCACAGCGAAGACCCCTCGTCCGAACAGAAACTGATCTCTGAAGA
AGACCTGTAA

Kell-VNA/B: Amino acid sequence of Kell-VNA/B, where VHH-D10 is indicated in bold, VHH-G10 is indicated in bold and underlining, GPA is indicated in italics, and the Myc tag is indicated in underlining:

(SEQ ID NO:8)
MEGGDQSEEEPRERSQAGGMGTLWSQESTPEERLPVEGSRPWAVARRVLTAILILGLLLC
FSVLLFYNFQNCGPRPCETSVCLDLRDHYLASGNTSVAPCTDFFSFACGRAKETNNSFQEL
ATKNKNRLRRILEVQNSWHPGSGEEKAFQFYNSCMDTLAIEAAGTGPLRQVIEELGGWRIS
GKWTSLNFNRTLRLLMSQYGHFPFFRAYLGPHPASPHTPVIQIDQPEFDVPLKQDQEQKI
YAQIFREYLTYLNQLGTLLGGDPSKVQEHSSLSISITSRLFQFLRPLEQRRAQGKLFQMVTI
DQLKEMAPAIDWLSCLQATFTPMSLSPSQSLVVHDVEYLKNMSQLVEEMLLKQRDFLQS
HMILGLVVTLSPALDSQFQEARRKLSQKLRELTEQPPMPARPRWMKCVEETGTFFEPTLA
ALFVREAFGPSTRSAAMKLFTAIRDALITRLRNLPWMNEETQNMAQDKVAQLQVEMGASE
WALKPELARQEYNDIQLGSSFLQSVLSCVRSLRARIVQSFLQPHPQHRWKVSPWDVNAYYS
VSDHVVVFPAGLLQPPFFHPGYPRAVNFGAAGSIMAHELLHIFYQLLLPGGCLACDNHAL
QEAHLCLKRHYAAFPLPSRTSFNDSLTFLENAADVGGLAIALQAYSKRLLRHHGETVLPSL
DLSPQQIFFRSYAQVMCRKPSPQDSHDTHSPPHLRVHGPLSSTPAFARYFRCARGALLNPS
SRCQLWQGVQAQLQLVESGGGLVQPGGSLRLSCAASGFTLDSYAIGWFRQAPGKEREGVAC
ISASGSGTDYVDSVKGRFTVSRDQAKSMVFLQMNNMKPEDAAVYYCAADYRPRPLPIQAPCT

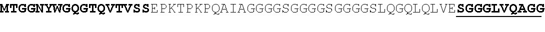

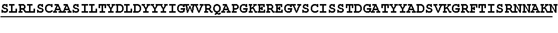

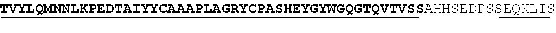

EEDL

### Generating Red Blood Cells Expressing Fusion Proteins

Also described herein are methods of preparing genetically engineered red blood cells capable of expressing proteins of interest, such as cell surface proteins or fusion proteins comprising a red blood cell transmembrane protein and an antigen binding protein that binds to an antigen, such as a toxin or an antigen of a pathogen. Such engineered red blood cells may be produced using any of the cell culture methods provided herein

### Genetic Modification of Progenitor Cells

Expression vectors for producing proteins of interest (e.g., fusion proteins) may be introduced into CD34⁺ progenitor cells, which can be isolated from an original source or obtained from the expansion stage described above via routine recombinant technology. In some instances, the expression vectors can be designed such that they can incorporate into the genome of cells by homologous or non-homologous recombination by methods known in the art. Methods for transferring expression vectors into CD34⁺ progenitor cells include, but are not limited to, viral mediated gene transfer, liposome mediated transfer, transformation, transfection and transduction, e.g., viral mediated gene transfer such as the use of vectors based on DNA viruses such as adenovirus, adeno-associated virus and herpes virus, as well as retroviral based vectors. Examples of modes of gene transfer include e.g., naked DNA, CaPO4 precipitation, DEAE dextran, electroporation, protoplast fusion, lipofection, cell microinjection, and viral vectors, adjuvant-assisted DNA, gene gun, catheters. In one example, a viral vector is used. To enhance delivery of non-viral vectors to a cell, the nucleic acid or protein can be conjugated to antibodies or binding fragments thereof which bind cell surface antigens, e.g., CD34. Liposomes that also include a targeting antibody or fragment thereof can be used in the methods described herein.

A “viral vector” as described herein refers to a recombinantly produced virus or viral particle that comprises a polynucleotide to be delivered into a host cell, either in vivo, ex vivo or in vitro. Examples of viral vectors include retroviral vectors such as lentiviral vectors, adenovirus vectors, adeno-associated virus vectors and the like. In aspects where gene transfer is mediated by a retroviral vector, a vector construct refers to the polynucleotide comprising the retroviral genome or part thereof, and a therapeutic gene.

A gene encoding any protein, including a cell surface protein (e.g., transmembrane protein, integral membrane protein), or any of the fusion proteins provided herein, can be inserted into a suitable vector (e.g., a retroviral vector) using methods well known in the art. Sambrook et al., Molecular Cloning, A Laboratory Manual, 3rd Ed., Cold Spring Harbor Laboratory Press. For example, the gene and vector can be contacted, under suitable conditions, with a restriction enzyme to create complementary ends on each molecule that can pair with each other and be joined together with a ligase. Alternatively, synthetic nucleic acid linkers can be ligated to the termini of a gene. These synthetic linkers contain nucleic acid sequences that correspond to a particular restriction site in the vector. Additionally, the vector can contain, for example, some or all of the following: a selectable marker gene, such as the neomycin gene for selection of stable or transient transfectants in mammalian cells; enhancer/promoter sequences from the immediate early gene of human CMV for high levels of transcription; transcription termination and RNA processing signals from SV40 for mRNA stability; SV40 polyoma origins of replication and ColE 1 for proper episomal replication; versatile multiple cloning sites; and T7 and SP6 RNA promoters for in vitro transcription of sense and antisense RNA. Suitable vectors and methods for producing vectors containing transgenes are well known and available in the art. Sambrook et al., Molecular Cloning, A Laboratory Manual, 3rd Ed., Cold Spring Harbor Laboratory Press.

In some embodiments, any of the CD34⁺ progenitor cells provided herein (e.g., produced by any of the cell culture methods provided herein) may express one or more proteins of interest, which may be fused to a red blood cell transmembrane protein. In some embodiments, the protein of interest is an antibody. Exemplary antibodies include, but are not limited to, Abciximab (glycoprotein IIb/IIIa; cardiovascular disease), Adalimumab (TNF-α, various auto-immune disorders, e.g., rheumatoid arthritis), Alemtuzumab (CD52; chronic lymphocytic leukemia), Basiliximab (IL-2Rα receptor (CD25); transplant rejection), Bevacizumab (vascular endothelial growth factor A; various cancers, e.g., colorectal cancer, non-small cell lung cancer, glioblastoma, kidney cancer; wet age-related macular degeneration), Catumaxomab, Cetuximab (EGF receptor, various cancers, e.g., colorectal cancer, head and neck cancer), Certolizumab (e.g., Certolizumab pegol) (TNF alpha; Crohn's disease, rheumatoid arthritis), Daclizumab (IL-2Rα receptor (CD25); transplant rejection), Eculizumab (complement protein C5; paroxysmal nocturnal hemoglobinuria), Efalizumab (CD11a; psoriasis), Gemtuzumab (CD33; acute myelogenous leukemia (e.g., with calicheamicin)), Ibritumomab tiuxetan (CD20; Non-Hodgkin lymphoma (e.g., with yttrium-90 or indium-111)), Infliximab (TNF alpha; various autoimmune disorders, e.g., rheumatoid arthritis) Muromonab-CD3 (T Cell CD3 receptor; transplant rejection), Natalizumab (alpha-4 (α4) integrin; multiple sclerosis, Crohn's disease), Omalizumab (IgE; allergy-related asthma), Palivizumab (epitope of RSV F protein; Respiratory Syncytial Virus infection), Panitumumab (EGF receptor; cancer, e.g., colorectal cancer), Ranibizumab (vascular endothelial growth factor A; wet age-related macular degeneration) Rituximab (CD20; Non-Hodgkin lymphoma), Tositumomab (CD20; Non-Hodgkin lymphoma), Trastuzumab (ErbB2; breast cancer), and any antigen-binding fragment thereof.

In some embodiments, the protein of interest is a cytokine, e.g., a type I cytokine. In some embodiments the protein of interest is a four-helix bundle protein, e.g., a four-helix bundle cytokine. Exemplary four-helix bundle cytokines include, e.g., certain interferons (e.g., a type I interferon, e.g., IFN-α), interleukins (e.g., IL-2, IL-3, IL-4, IL-5, IL-6, IL-7, IL-12), and colony stimulating factors (e.g., G-CSF, GM-CSF, M-CSF). The IFN can be, e.g., interferon alpha 2a or interferon alpha 2b. See, e.g., Mott H R and Campbell I D. “Four-helix bundle growth factors and their receptors: protein-protein interactions.” Curr Opin Struct Biol. 1995 February; 5(1):114-21; Chaiken I M, Williams W V. “Identifying structure-function relationships in four-helix bundle cytokines: towards de novo mimetics design.” Trends Biotechnol. 1996 October; 14(10):369-75; Klaus W, et al., “The three-dimensional high resolution structure of human interferon alpha-2a determined by heteronuclear NMR spectroscopy in solution”. J. Mol Biol., 274(4):661-75, 1997, for further discussion of certain of these cytokines.

The protein of interest may also be a cytokine protein that has a similar structure to one or more of the afore-mentioned cytokines. For example, the cytokine can be an IL-6 class cytokine such as leukemia inhibitory factor (LIF) or oncostatin M. In some embodiments, the cytokine is one that in nature binds to a receptor that comprises a GP130 signal transducing subunit. Other four-helix bundle proteins of interest include growth hormone (GH), prolactin (PRL), and placental lactogen. In some embodiments, the protein of interest is an erythropoiesis stimulating agent, e.g., (EPO), which is also a four-helix bundle cytokine. In some embodiments, an erythropoiesis stimulating agent is an EPO variant, e.g., darbepoetin alfa, also termed novel erythropoiesis stimulating protein (NESP), which is engineered to contain five N-linked carbohydrate chains (two more than recombinant HuEPO). In some embodiments, the protein comprises five helices. For example, the protein can be an interferon beta, e.g., interferon beta-1a or interferon beta-1b, which (as will be appreciated) is often classified as a four-helix bundle cytokine. In some embodiments, the protein of interest is IL-9, IL-10, IL-11, IL-13, or IL-15. See, e.g., Hunter, C A, Nature Reviews Immunology 5, 521-531, 2005, for discussion of certain cytokines. See also Paul, W E (ed.), Fundamental Immunology, Lippincott Williams & Wilkins; 6th ed., 2008. Any protein described in the references cited herein, all of which are incorporated herein by reference, can be used as a protein of interest.

In addition, the protein of interest may be a protein that is approved by the US Food & Drug Administration (or an equivalent regulatory authority such as the European Medicines Evaluation Agency) for use in treating a disease or disorder in humans. Such proteins may or may not be one for which a PEGylated version has been tested in clinical trials and/or has been approved for marketing.

The protein of interest may also be a neurotrophic factor, i.e., a factor that promotes survival, development and/or function of neural lineage cells (which term as used herein includes neural progenitor cells, neurons, and glial cells, e.g., astrocytes, oligodendrocytes, microglia). For example, in some embodiments, the protein of interest is a factor that promotes neurite outgrowth. In some embodiments, the protein is ciliary neurotrophic factor (CNTF; a four-helix bundle protein) or an analog thereof such as Axokine, which is a modified version of human Ciliary neurotrophic factor with a 15 amino acid truncation of the C terminus and two amino acid substitutions, which is three to five times more potent than CNTF in in vitro and in vivo assays and has improved stability properties.

In another example, the protein of interest is a protein that forms homodimers or heterodimers, (or homo- or heterooligomers comprising more than two subunits, such as tetramers). In certain embodiments the homodimer, heterodimer, or oligomer structure is such that a terminus of a first subunit is in close proximity to a terminus of a second subunit. For example, an N-terminus of a first subunit is in close proximity to a C-terminus of a second subunit. In certain embodiments the homodimer, heterodimer, or oligomer structure is such that a terminus of a first subunit and a terminus of a second subunit are not involved in interaction with a receptor, so that the termini can be joined via a non-genetically encoded peptide element without significantly affecting biological activity. In some embodiments, termini of two subunits of a homodimer, heterodimer, or oligomer are conjugated via click chemistry using a method described herein, thereby producing a dimer (or oligomer) in which at least two subunits are covalently joined. For example, the neurotrophins nerve growth factor (NGF); brain-derived neurotrophic factor (BDNF); neurotrophin 3 (NT3); and neurotrophin 4 (NT4) are dimeric molecules which share approximately 50% sequence identity and exist in dimeric forms. See, e.g., Robinson R C, et al., “Structure of the brain-derived neurotrophic factor/neurotrophin 3 heterodimer.”, Biochemistry. 34(13):4139-46, 1995; Robinson R C, et al., “The structures of the neurotrophin 4 homodimer and the brain-derived neurotrophic factor/neurotrophin 4 heterodimer reveal a common Trk-binding site.” Protein Sci. 8(12):2589-97, 1999, and references therein. In some embodiments, the dimeric protein is a cytokine, e.g., an interleukin.

Alternatively, the protein of interest is an enzyme, e.g., an enzyme that is important in metabolism or other physiological processes. As is known in the art, deficiencies of enzymes or other proteins can lead to a variety of disease. Such diseases include diseases associated with defects in carbohydrate metabolism, amino acid metabolism, organic acid metabolism, porphyrin metabolism, purine or pyrimidine metabolism, lysosomal storage disorders, blood clotting, etc. Examples include Fabry disease, Gaucher disease, Pompe disease, adenosine deaminase deficiency, asparaginase deficiency, porphyria, hemophilia, and hereditary angioedema. In some embodiments, a protein is a clotting or coagulation factor, (e.g., factor VII, VIIa, VIII or IX). In other embodiments a protein is an enzyme that plays a role in carbohydrate metabolism, amino acid metabolism, organic acid metabolism, porphyrin metabolism, purine or pyrimidine metabolism, and/or lysosomal storage, wherein exogenous administration of the enzyme at least in part alleviates the disease.

Moreover, the protein of interest may be a receptor or receptor fragment (e.g., extracellular domain). In some embodiments the receptor is a TNFα receptor. In certain embodiments, the protein of interest comprises urate oxidase.

In some embodiments, the protein of interest is an antigenic protein, which may derive from a pathogen, such as a virus or bacterium (e.g., any of the viruses or bacteria provided herein). An antigenic protein may be naturally occurring or synthetic in various embodiments. It may be naturally produced by and/or comprises a polypeptide or peptide that is genetically encoded by a pathogen, an infected cell, or a neoplastic cell (e.g., a cancer cell). In some examples, the antigenic protein is an autoantigen (“self antigen”), that has the capacity to initiate or enhance an autoimmune response. In other examples, the antigenic protein is produced or genetically encoded by a virus, bacteria, fungus, or parasite which, in some embodiments, is a pathogenic agent. In some embodiments, an agent (e.g., virus, bacterium, fungus, parasite) infects and, in some embodiments, causes disease in, at least one mammalian or avian species, e.g., human, non-human primate, bovine, ovine, equine, caprine, and/or porcine species. In some embodiments, a pathogen is intracellular during at least part of its life cycle. In some embodiments, a pathogen is extracellular. It will be appreciated that an antigen that originates from a particular source may, in various embodiments, be isolated from such source, or produced using any appropriate means (e.g., recombinantly, synthetically, etc.), e.g., for purposes of using the antigen, e.g., to identify, generate, test, or use an antibody thereto). An antigen may be modified, e.g., by conjugation to another molecule or entity (e.g., an adjuvant), chemical or physical denaturation, etc. In some embodiments, an antigen is an envelope protein, capsid protein, secreted protein, structural protein, cell wall protein or polysaccharide, capsule protein or polysaccharide, or enzyme. In some embodiments an antigen is a toxin, e.g., a bacterial toxin.

Other proteins of interest may be found in, e.g., U.S. Ser. Nos. 10/773,530; 11/531,531; U.S. Ser. Nos. 11/707,014; 11/429,276; 11/365,008, all of which are incorporated by reference herein. The invention encompasses application of the inventive methods to any of the proteins described herein and any proteins known to those of skill in the art.

Modification of CD34⁺ progenitor cells can comprise the use of an expression cassette created for either constitutive or inducible expression of the introduced gene. Such an expression cassette can include regulatory elements such as a promoter, an initiation codon, a stop codon, and a polyadenylation signal. The elements are preferably operable in the progenitor cells or in cells that arise from the progenitor cells (e.g., enucleated red blood cells) after administration (e.g., infusion) into an individual. Moreover, the elements can be operably linked to the gene encoding the protein of interest such that the gene is operational (e.g., is expressed) in the progenitor cells or red blood cells derived therefrom.

A variety of promoters can be used for expression of the fusion protein of interest. Promoters that can be used to express the protein are well known in the art. Promoters include cytomegalovirus (CMV) intermediate early promoter, a viral LTR such as the Rous sarcoma virus LTR, HIV-LTR, HTLV-1 LTR, the simian virus 40 (SV40) early promoter, E. coli lac UV5 promoter and the herpes simplex tk virus promoter.

Regulatable promoters can also be used. Such regulatable promoters include those using the lac repressor from E. coli as a transcription modulator to regulate transcription from lac operator-bearing mammalian cell promoters [Brown, M. et al., Cell, 49:603-612 (1987)], those using the tetracycline repressor (tetR) [Gossen, M., and Bujard, H., Proc. Natl. Acad. Sci. USA 89:5547-5551 (1992); Yao, F. et al., Human Gene Therapy, 9:1939-1950 (1998); Shockelt, P., et al., Proc. Natl. Acad. Sci. USA, 92:6522-6526 (1995)]. Other systems include FK506 dimer, VP16 or p65 using astradiol, RU486, diphenol murislerone or rapamycin. Inducible systems are available from Invitrogen, Clontech and Ariad.

Regulatable promoters that include a repressor with the operon can be used. In one embodiment, the lac repressor from E. coli can function as a transcriptional modulator to regulate transcription from lac operator-bearing mammalian cell promoters [M. Brown et al., Cell, 49:603-612 (1987)]; Gossen and Bujard (1992); [M. Gossen et al., Natl. Acad. Sci. USA, 89:5547-5551 (1992)] combined the tetracycline repressor (tetR) with the transcription activator (VP 16) to create a tetR-mammalian cell transcription activator fusion protein, tTa (tetR-VP 16), with the tetO-bearing minimal promoter derived from the human cytomegalovirus (hCMV) major immediate-early promoter to create a tetR-tet operator system to control gene expression in mammalian cells. In one embodiment, a tetracycline inducible switch is used. The tetracycline repressor (tetR) alone, rather than the tetR-mammalian cell transcription factor fusion derivatives can function as potent trans-modulator to regulate gene expression in mammalian cells when the tetracycline operator is properly positioned downstream for the TATA element of the CMVIE promoter [F. Yao et al., Human Gene Therapy, supra]. One particular advantage of this tetracycline inducible switch is that it does not require the use of a tetracycline repressor-mammalian cells transactivator or repressor fusion protein, which in some instances can be toxic to cells [M. Gossen et al., Natl. Acad. Sci. USA, 89:5547-5551 (1992); P. Shockett et al., Proc. Natl. Acad. Sci. USA, 92:6522-6526 (1995)], to achieve its regulatable effects.

The effectiveness of some inducible promoters can be increased over time. In such cases one can enhance the effectiveness of such systems by inserting multiple repressors in tandem, e.g., TetR linked to a TetR by an internal ribosome entry site (IRES). Alternatively, one can wait at least 3 days before screening for the desired function. While some silencing may occur, it can be minimized by using a suitable number of cells, preferably at least 1×10⁴, more preferably at least 1×10⁵, still more preferably at least 1×10⁶, and even more preferably at least 1×10⁷. One can enhance expression of desired proteins by known means to enhance the effectiveness of this system. For example, using the Woodchuck Hepatitis Virus Posttranscriptional Regulatory Element (WPRE). See Loeb, V. E., et al., Human Gene Therapy 10:2295-2305 (1999); Zufferey, R., et al., J. of Virol. 73:2886-2892 (1999); Donello, J. E., et al., J. of Virol. 72:5085-5092 (1998).

Examples of polyadenylation signals useful to practice the methods described herein include, but are not limited to, human collagen I polyadenylation signal, human collagen II polyadenylation signal, and SV40 polyadenylation signal.

The exogenous genetic material that includes the protein-encoding gene (e.g., gene encoding any of the fusion proteins provided herein) operably linked to the regulatory elements may remain present in the cell as a functioning cytoplasmic molecule, a functioning episomal molecule or it may integrate into the cell's chromosomal DNA. Exogenous genetic material may be introduced into cells where it remains as separate genetic material in the form of a plasmid. Alternatively, linear DNA, which can integrate into the chromosome, may be introduced into the cell. When introducing DNA into the cell, reagents, which promote DNA integration into chromosomes, may be added. DNA sequences, which are useful to promote integration, may also be included in the DNA molecule. Alternatively, RNA may be introduced into the cell.

Selectable markers can be used to monitor uptake of the desired transgene into the progenitor cells described herein. These marker genes can be under the control of any promoter or an inducible promoter. These are known in the art and include genes that change the sensitivity of a cell to a stimulus such as a nutrient, an antibiotic (e.g., apmicillin), etc. Genes include those for neo, puro, tk, multiple drug resistance (MDR), etc. Other genes express proteins that can readily be screened for such as green fluorescent protein (GFP), blue fluorescent protein (BFP), luciferase, and LacZ.

### Genetic Modification for Expressing Proteins on a Cell Surface

In some embodiments, the CD34⁺ progenitor cells provided herein can be genetically modified using any of the methods described herein or known in the art such that they are capable of expressing a protein, such as a cell surface protein (e.g., integral membrane protein or transmembrane protein), or a fusion protein comprising a red blood cell transmembrane protein and a peptide heterologous to the membrane protein, such as an antigen binding protein that binds to a toxin or antigen of a pathogen. A red blood cell transmembrane protein can be conjugated to another peptide (e.g., antigen binding protein) directly or via a linker. Preferably the transduction of the CD34⁺ progenitor cells with a gene encoding a surface protein is via a viral vector such as a retroviral vector (as described in for example, in WO 94/29438, WO 97/21824 and WO 97/21825).

A cell surface protein can be a fusion protein comprising a membrane protein and at least one heterologous protein (e.g., antigen binding protein). In some embodiments, the cell surface protein comprises a red blood cell transmembrane protein and an antigen binding protein. In some embodiments, a fusion protein for use in the methods described herein, which may present on mature RBCs, inhibit neither erythroid differentiation nor be targeted for degradation during the extensive membrane remodeling that occurs during enucleation and at the later reticulocyte stage. Such membrane proteins are known in the art. See, e.g., Liu et al., 2010, Blood, 115(10):2021-2027. For example, red blood cell precursors express high levels of the transferrin receptor (Tfr), a type II membrane protein. Any membrane proteins present in mature RBCs can be used for constructing the fusion proteins as described herein. The membrane protein can be fused to an antigen binding protein at the terminus that is exposed to the extracellular or luminal space. In some examples, the terminus of the fusion protein that is exposed to cytoplasm may also be fused to a protein of interest, which can be a cytoplasmic protein.

In one example, the cell surface protein expressed by the CD34⁺ cell (e.g., CD34⁺ cells produced by any of the cell culture methods provided herein) is a fusion protein comprising a type I red blood cell transmembrane protein, such as glycophorin A, intercellular adhesion molecule 4 (ICAM-4), Lutheran glycoprotein (CD329), Basigin (CD147), and an antigen binding protein. In some embodiments, the transmembrane proteins can are human proteins. However, the transmembrane proteins may be from another organism, such as mouse, rat, monkey, dog, pig or cow. Type I transmembrane proteins are single-pass transmembrane proteins which have their N-termini exposed to the extracellular or luminal space. In another example, the cell surface protein is a fusion protein comprising a type II red blood cell transmembrane protein, e.g., Kell, or CD71, and an antigen binding protein (e.g., sortase A). Type II transmembrane proteins are single-pass transmembrane proteins which have their C-termini exposed to the extracellular or luminal space. In another example, the cell surface protein is a fusion protein comprising a type III red cell transmembrane protein and an antigen binding protein. Type III membrane proteins are multi-pass structures, which usually have their N-termini exposed to the extracellular or luminal space. Examples of type III red cell transmembrane proteins include, without limitation, GLUT1, Aquaporin 1, and Band 3. In one example, a type III transmembrane protein can be fused with an antigen binding protein at the N-terminus of the transmembrane protein, or at the C-terminus of the transmembrane protein.

When necessary, the fusion protein of a CD34⁺ progenitor cell can further include additional suitable tags, which include, but are not limited to, amino acids, nucleic acids, polynucleotides, sugars, carbohydrates, polymers, lipids, fatty acids, and small molecules. Other suitable tags will be apparent to those of skill in the art and the invention is not limited in this aspect.

In some embodiments, such a tag comprises a sequence useful for purifying, expressing, solubilizing, and/or detecting a polypeptide. In some embodiments, a tag can serve multiple functions. In some embodiments, the tag is relatively small, e.g., ranging from a few amino acids up to about 100 amino acids long. In some embodiments, a tag is more than 100 amino acids long, e.g., up to about 500 amino acids long, or more. In some embodiments, a tag comprises an HA, TAP, Myc, 6×His, Flag, streptavidin, biotin, or GST tag, to name a few examples. In some embodiments, a tag comprises a solubility-enhancing tag (e.g., a SUMO tag, NUS A tag, SNUT tag, or a monomeric mutant of the Ocr protein of bacteriophage T7). See, e.g., Esposito D and Chatterjee D K. Curr Opin Biotechnol.; 17(4):353-8 (2006). In some embodiments, a tag is cleavable, so that it can be removed, e.g., by a protease. In some embodiments, this is achieved by including a protease cleavage site in the tag, e.g., adjacent or linked to a functional portion of the tag. Exemplary proteases include, e.g., thrombin, TEV protease, Factor Xa, PreScission protease, etc. In some embodiments, a “self-cleaving” tag is used. See, e.g., Wood et al., International PCT Application PCT/US2005/05763, filed on Feb. 24, 2005, and published as WO/2005/086654 on Sep. 22, 2005.

In some embodiments, the fusion protein of a CD34⁺ progenitor cell comprises a red blood cell transmembrane protein, a first antigen binding protein, and a second antigen binding protein. In some embodiments, the first antigen binding protein and the second antigen binding protein bind to different epitopes of the same antigen. In some embodiments, the first antigen binding protein and the second antigen binding protein bind to the same epitope of the same antigen. In some embodiments, the first antigen binding protein and/or the second antigen binding protein bind to a toxin or an antigen of a pathogen. In some embodiments, the fusion protein expressed by a CD34⁺ progenitor cell is any of the fusion proteins provided herein.

Any of the genetically modified CD34⁺ progenitor cells described herein can be cultured under suitable conditions allowing for differentiation into mature enucleated red blood cells, e.g., the in vitro culturing process described herein. The resultant enucleated red blood cells are capable of expressing the surface protein of interest, such as a fusion protein as described herein, which can be evaluated and confirmed by routine methodology (e.g., Western blotting or FACS analysis).

### Uses of Red Blood Cells

Any of the genetically modified CD34⁺ progenitor cells and enucleated red blood cells (including those obtained from the in vitro culturing process described herein), which can also be genetically modified as described herein, are within the scope of the present disclosure.

Enucleated red blood cells having a surface modification of an agent of interest (e.g., a fusion protein comprising a red blood cell transmembrane protein and an antigen binding protein) as described herein can be administered to a subject in need thereof (e.g., a human patient) for various purposes, e.g., treating a specific disease when the agent of interest is a therapeutic agent, performing a blood transfusion, detecting the presence of specific cell types when the agent of interest is capable of recognizing the target cells, eliciting desired immune responses when the agent of interest is immunogenic, and neutralizing a toxin in a subject when the agent is capable of binding to the toxin. Any suitable delivery route can be used in the methods described herein, e.g., cell infusion.

The term “subject,” as used herein, refers to an individual organism, for example, an individual mammal. In some embodiments, the subject is a human. In some embodiments, the subject is a non-human mammal. In some embodiments, the subject is a non-human primate. In some embodiments, the subject is a rodent. In some embodiments, the subject is a sheep, a goat, a cattle, a cat, or a dog. In some embodiments, the subject is a vertebrate, an amphibian, a reptile, a fish, an insect, a fly, or a nematode. In some embodiments, the subject is a research animal. In some embodiments, the subject is genetically engineered, e.g., a genetically engineered non-human subject. In some embodiments, the subject is a healthy volunteer. The subject may be of either sex and at any stage of development.

In some examples, the red blood cells are delivered to the same subject from which the cells are originally obtained. For example, peripheral blood cells can be obtained from a subject such as human patient, expanded in vitro (e.g., using the cell culture methods provided herein), genetically modified such that they express proteins of interest (e.g., fusion proteins comprising a red blood cell transmembrane protein and an antigen binding protein), and differentiated into enucleated red blood cells. The modified enucleated red blood cells can then be administered to the same subject.

In other examples, the CD34⁺ progenitor cells are obtained from a suitable subject and, after being differentiated and genetically modified as described herein, the resultant enucleated red blood cells are administered to a different subject, which preferably is immunocompatible with the donor (e.g., administration of the enucleated blood cells would not elicit undesirable immune responses).

The enucleated red blood cells can be administered to a subject who has or suspected of having a condition associated with red blood cell deficiency, for example, anemia, blood loss due to surgery or trauma. The enucleated red blood cells can also be administered to a subject in need of a treatment or diagnosis that can be achieved by the agent of interest conjugated on the surface of the enucleated red blood cells. For example, the enucleated red blood cells may be conjugated to a cancer antigen or an antigen derived from a pathogen. Such cells can be delivered to a subject in need (e.g., a human subject having or at risk for cancer or infection by the pathogen) for either preventive or therapeutic treatment.

In another example, the enucleated red blood cells express on their surface an antigen binding protein. In some embodiments, the antigen binding protein is fused to a red blood cell transmembrane protein, which is attached to the membrane of a red blood cell. In some embodiments, the antigen binding protein is an antigen binding protein that binds to a toxin, which include natural and synthetic toxins. It should be appreciated that cells expressing such antigen binding proteins can be used to neutralize the toxin (e.g., in a subject) to which the antigen binding protein binds. Cells may express one or more antigen binding proteins that bind to any of the toxins provided herein. In some embodiments, the toxin is a biotoxin. Examples of biotoxins include, without limitation cyanotoxins (e.g., produced by cyanobacteria), dinotoxins, (e.g., produced by dinoflagellates), necrotoxins (e.g., toxins from spiders, snakes or necrotizing fasciitis), neurotoxins (e.g., toxins from scorpions, black widow spiders, box jellyfish, elapid snakes, cone snails, blue-ringed octopi, venomous fish, frogs, palythoa coral, algae, cyanobacteria and dinoflagellates), myotoxins (e.g., toxins from rattlesnakes and eastern bearded dragons), and cytotoxins (e.g., ricin, apitoxin and T-2 mycotixin). In some embodiments, the antigen binding protein binds to a toxin selected from the group consisting of botulinum toxin, tetanus toxin, diphtheria toxin, dioxin, muscarine, bufotoxin, sarin, anthrax toxin, shiga toxin (e.g., naturally produced by Shigella dysenteriae), Shiga-like toxins 1 and 2 (e.g., naturally produced by certain strains of Escherichia Coli), pertussis toxin (e.g., naturally produced by Bordetealla pertussis), one or more components of a venom (e.g., a snake, spider, scorpion jellyfish or wasp), and ricin. It should be appreciated, however, that antigen binding proteins may be designed to bind other toxins and are within the scope of this disclosure. As demonstrated in the Examples section, red blood cells that express antigen binding proteins that bind toxins are useful for neutralizing and/or mitigating the negative effects (e.g., lethality) of such toxins in an organism.

In another example, the enucleated red blood cells express on their surface an antigen binding protein that binds an antigen of a pathogen (e.g., an antigen of a virus, bacterium, protozoa, prion, fungus or other micro-organism). As used herein, the term “pathogen” refers to an agent that is capable of producing a disease in an organism. A skilled artisan would understand that red blood cells expressing antigen binding proteins that bind such pathogens would be useful for neutralizing and/or mitigating the negative effects (e.g., disease progression) of such pathogens in an organism. In some embodiments, the binding protein binds an antigen of an bacterium, for example, an antigen from any of the bacteria provided herein. Exemplary bacterium include, without limitation, those listed above in the Fusion Protein section. In some embodiments, the binding protein binds an antigen of a virus, for example, an antigen from any of the viruses provided herein. Exemplary viruses include, without limitation, those listed above in the Fusion Protein section. In some embodiments, the binding protein binds a tumor antigen, for example, any of the tumor antigens provided herein. Exemplary tumor antigens include, without limitation, those listed above in the Fusion Protein section.

In another example, the enucleated red blood cells can express on their surface an enzyme effective in treating a disease or disorder associated with deficiency of the enzyme, e.g., Fabry disease, Gaucher disease, Pompe disease, adenosine deaminase deficiency, asparaginase deficiency, porphyria, hemophilia, and hereditary angioedema.

In other examples, the enucleated red blood cells carry a clotting or coagulation factor, (e.g., factor VII, VIIa, VIII or IX) and may be delivered to a human subject having or suspected of having conditions associated with abnormal blood clotting or coagulation.

### Kits

Some aspects of the present disclosure provide kits useful for the culturing and/or genetic modification and surface modification of red blood cells. Such a kit can comprise one or more expression vectors encoding one or more fusion protein each comprising a red blood cell membrane protein and a peptide (e.g., antigen binding protein) as described herein. If a kit comprises multiple expression vectors as described, they can include sequences encoding different red blood cell transmembrane proteins and/or antigen binding proteins. In some embodiments, the fusion proteins may comprise more than one (e.g., two, three, four, five, or more) antigen binding proteins that may bind the same or different epitopes of an antigen.

Alternatively or in addition, the kits described herein can comprise one or more of the medium components for use in the in vitro culturing process for producing enucleated red blood cells, e.g., one or more of the cytokines used therein, and one or more of the media used therein, and/or other components for cell culture. In some embodiments, the kit comprises an expansion medium (e.g., any of the expansion media provided herein). In some embodiments, the kit comprises one or more differentiation media, such as differentiation medium 1, differentiation medium 2, differentiation medium 3, differentiation medium 4, and/or differentiation medium 5. It should be appreciated that the kit may comprise one or more of any of the differentiation media provided herein.

In some embodiments, the kit further comprises a buffer or reagent useful for carrying out the cell culture methods or genetic modification of cells, for example, a buffer or reagent described in the Examples section.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

In order that the invention described herein may be more fully understood, the following examples are set forth. The examples described in this application are offered to illustrate the methods, compositions, and systems provided herein and are not to be construed in any way as limiting their scope.

### Example 1. Genetically Engineered Red Cells Expressing Single Domain Camelid Antibodies Confer Long-Term Protection Against Botulinum Neurotoxin

### Abstract

A short half-life in the circulation limits the application of therapeutics such as single domain antibodies (VHHs). Red blood cells (RBCs) are utilized to prolong the circulatory half-life of VHHs. Here VHHs against botulinum neurotoxin A (BoNT/A) are presented on the surface of RBCs by expressing chimeric proteins of VHHs with Glycophorin A or Kell. Mice whose RBCs carry the chimeric proteins exhibit resistance to 10,000 times the lethal dose (LD50) of BoNT/A, and transfusion of these RBCs into naïve mice affords protection for up to 28 days. An improved CD34⁺ culture system is further utilized to engineer human RBCs that express these chimeric proteins. Mice transfused with these RBCs are resistant to highly lethal doses of BoNT/A. The disclosed work demonstrates that engineered RBCs expressing VHHs can provide prolonged prophylactic protection against bacterial toxins, and illustrates the potentially broad translatability of the present strategy for therapeutic applications.

### Introduction

Botulinum neurotoxin serotype A (BoNT/A) is chosen as a model toxin due to its importance as both a source of food poisoning and a potential bioweapon, and the robust tools available for evaluating and quantifying antitoxin therapeutic efficacy. BoNT/A targets neurons and inhibits the release of neurotransmitters from pre-synaptic terminals by cleaving synaptosomal-associated protein of 25 kDa (SNAP25), a member of the soluble N-ethylmaleimide-sensitive factor-attachment protein receptor (SNARE) protein family⁹. Blocking neurotransmitter release at the neuromuscular junction leads to flaccid paralysis and death¹⁰. Because of its extreme potency and availability, BoNT/A is considered a category A bioweapon by the Centers for Diseases Control and Prevention (CDC)¹⁰. Development and testing of potent antitoxin VNAs for treating BoNT/A botulism has been previously reported^{7, 11}. It has also been shown that appending an albumin-binding peptide to the C′-terminus of VHH anti-botulinum increases the serum half-life of this VHH to 1-2 days.¹¹ Other groups have also administered a combination of biotinylated VHH anti-BoNT/A and a fusion protein consisting of a scFv specific to murine glycophorin A and streptavidin. This strategy increases the VHH retention time in the circulation as well as its neutralization potency in mice. Nevertheless, neutralization protection only lasts for a maximum of four days, indicating the short-lived stability of the complex¹².

Aiming to improve the half-life of VHHs and VNAs in the circulation in vivo, these agents are covalently linked to proteins on the surface of red blood cells (RBCs), the most abundant cell type in the human body. Because RBCs have a circulatory half-life of 120 days in humans and ˜30-50 days in mice, attached cargoes can potentially circulate for weeks unless modified RBCs are cleared more rapidly. RBCs also possess a natural biocompatibility and a large surface area, allowing covalent attachment of large numbers of cargoes without provoking adverse immune reactions^{13, 14, 15}. Indeed, RBC transfusion has been carried out for centuries with negligible side effects. Lastly, RBCs lack nuclei, eliminating risks associated with administration of nucleated cells, especially those previously subjected to genetic manipulation^{14,16}.

Several techniques have been developed to attach therapeutic cargoes to the surface of RBCs¹⁷. Covalent chemical attachment provides strong cargo binding but chemical medication methods are not specific and may alter membrane properties and blood antigens^{18, 19}. Antibody mediated binding to RBCs has also been employed, though cargo dissociate over time²⁰. To preserve the native biological properties of RBCs and to seek to prolong the circulation time of VHHs, a different strategy was developed in which virus vectors encoding chimeric proteins were generated with one or more VHHs fused in frame to a cDNA encoding the RBC membrane proteins Glycophorin A or Kell. By expressing these cDNAs in murine RBC progenitors and generating RBCs either by in vitro culture or stem cell transplantation, it is shown that the half-life of anti-BoNT VHHs can be extended to several weeks, equal to that of unmodified RBCs. These anti-BoNT VHHs are functional; they bind, neutralize and remove BoNT/A from the circulation. Mice transfused with engineered RBCs accounting for less than 1% of total RBCs are resistant to multiple lethal doses of BoNT. Finally, it is demonstrated that human RBCs can be produced in culture and engineered to express chimeric proteins containing anti-BoNT VHHs. Transfusion of these engineered RBCs into mice protects them from death caused by BoNT challenge. It is suggested that similar types of engineered human RBCs can be used to provide long term protection against exposures to a variety of bacterial toxins and harmful viruses.

### Results

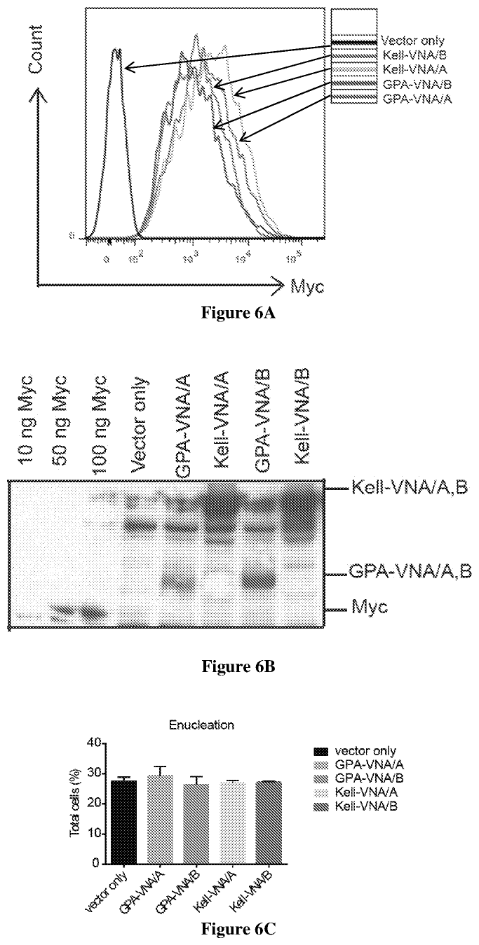

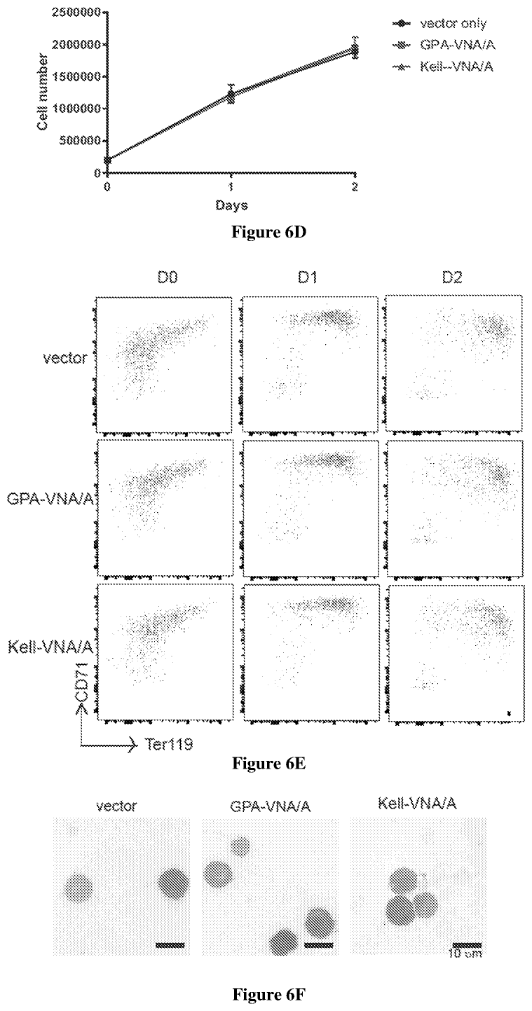

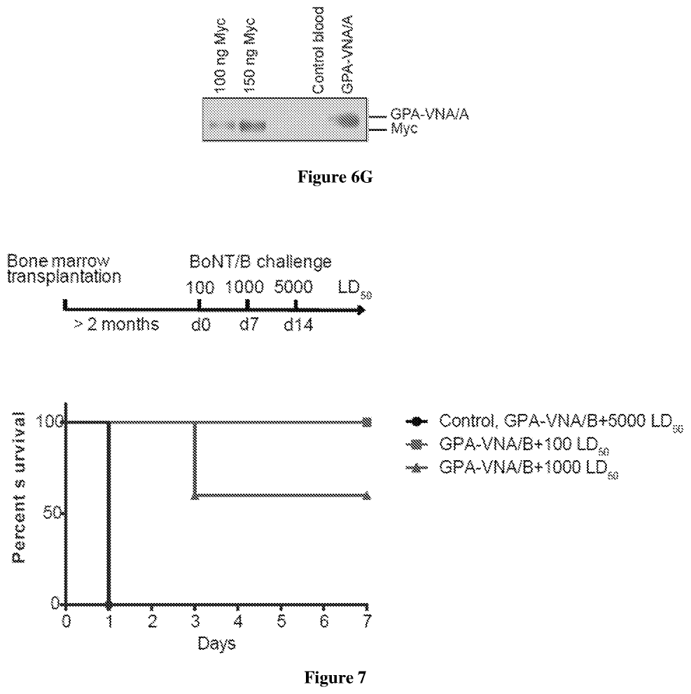

Retroviral constructs were first generated that can infect red cell progenitors and lead to expression of chimeric proteins on the surface of mature RBCs. Glycophorin A and Kell are chosen as the RBC membrane protein targets and genetically fused antitoxin VHHs at the N′-terminus of GPA and at the C′-terminus of Kell, respectively, to expose the VHHs on the external surface of the RBC plasma membrane (FIG. 1A) Both BoNT serotype A (BoNT/A) and serotype B (BoNT/B) are extremely toxic to humans; the two bispecific VNAs, termed VNA/A and VNA/B, recognize and neutralize BoNT/A and BoNT/B, respectively. VNA/A and VNA/B are heterodimers of two linked VHHs that recognize different epitopes on BoNT/A (ciA-H7 and ciA-B5)³ or BoNT/B (JLU-D10 and JLI-G10). The two VHHs are separated by a (GGGGS)₃ flexible spacer, and a myc-tag was added to the N′-terminus of GPA-VNA and the C′-terminus of Kell-VNA to simplify analysis. E14.5 mouse fetal liver red cell progenitors were infected with these retroviruses as well as with an empty vector as control. The progenitors were then cultured in vitro to differentiate into reticulocytes. Terminally differentiated cells express the chimeric GPA or Kell proteins on their surface, as indicated by myc surface expression (FIG. 6A). Each RBC was estimated to express ˜4,600,000 copies of GPA-VNA and 2,200,000 copies of Kell-VNA proteins per cell (Figure FIG. 6B; see legend to FIG. 6B for calculation). In this in vitro mouse fetal liver culture system, the VNA-expressing cells undergo enucleation at a level similar to control cells and have similar CD71 and Ter119 surface expression, proliferation, and morphology compared to control cells, suggesting that these modifications do not disturb normal red cell differentiation (FIGS. 6C-6F).

The ability of these engineered RBCs to neutralize BoNT/A was evaluated using in vitro neutralization assays (FIG. 1B). Primary rat neurons were co-incubated with BoNT/A and various amounts of engineered RBCs. More than 70% of SNAP25 was cleaved in control cultures, while SNAP25 was mostly intact in neurons co-incubated with 1,000,000 or 100,000 GPA-VNA/A-expressing RBCs. Incubation with 1,000,000 or 100,000 Kell-VNA/A-expressing cells also protected neurons from BoNT/A. The protective ability of the GPA-VNA/A and Kell-VNA/A was specific since there was no protection conferred by RBCs with surface expressed VNA/B.

Since the present engineered RBC progenitors undergo normal erythropoiesis in vitro, engineered RBCs are next produced in vivo by transplanting irradiated mice with fetal liver red cell progenitors engineered to express GPA-VNA/A or Kell-VNA/A. Complete blood counts (CBC) and myc surface expression (indicating the expression of the GPA-VNA/A chimera) from one batch of GPA-VNA/A transplanted mice (n=4) was followed over time (FIGS. 1C-1D). Compared to 7 week old female mice, the blood parameters of the transplanted mice varied, but were still within the normal range reported in the Mouse Phenome Database on the Jackson Laboratory website. Moreover, the average myc surface expression was stable over six months. In a separate batch of mice transplanted with cells expressing GPA-VNA/A, 2.96±0.10% of the RBCs were myc⁺, and mice transplanted with cells expressing the Kell-VNA/A chimera had 14.26±0.25% myc⁺ RBCs after six week bone marrow reconstitution. As shown in FIG. 6G, each of these RBCs contained about 3,100,000 VNA/A proteins per cell, about 1/15^{th} that found on red cells made in the in vitro culture.

These mice were then challenged with BoNT/A. As shown in FIG. 1E, both GPA-VNA/A and Kell-VNA/A producing mice, but not wild type mice, survived a 10 LD₅₀ BoNT/A challenge. Seven days later, these surviving mice were challenged with a 100 LD₅₀ of BoNT/A; both GPA-VNA/A and Kell-VNA/A mice survived. The mice were then challenged at weekly intervals with higher doses of BoNT/A. The Kell-VNA/A-expressing mice died at 250 LD₅₀; remarkably, the GPA-VNA/A mice survived even when challenged with 10,000 LD₅₀ BoNT/A.

The protective capacity of the engineered RBCs is versatile, since 60% of mice with 4.77±1.18% GPA-VNA/B chimera-expressing RBCs survived a BoNT/B challenge as high as 1000 LD₅₀ (FIG. 7) and 100% of mice expressing the GPA-VNA/B chimera survived a 100 LD₅₀ BoNT/B challenge.

Since red cells expressing the GPA-VNA/A chimeras seem to provide more potent protection in vivo than do Kell-VNA/A mice, the subsequent experiments were carried out utilizing GPA as the RBC membrane anchor proteins for the chimeric proteins.

It is likely that this potent protection stems from the continuous replenishment of engineered RBCs by the bone marrow of the reconstituted mice. Transfusion of engineered RBCs is a more realistic option to exploit engineered RBCs therapeutically in humans. Therefore, wild type mice were transfused with 100 μl of blood drawn from chimeric mice in which 6.22±0.25% of the RBCs displayed surface GPA-VNA/A. As there are 10⁷ cells/μl of whole blood, 6.22*10⁷ GPA-VNA/A expressing RBCs were therefore injected. The recipient mice were then challenged with BoNT/A (FIG. 2A). Blood containing GPA-VNA/A RBCs protected mice from 10 LD₅₀ BoNT/A, although these mice died within 24 hours if challenged with 100 LD₅₀ or higher doses of BoNT/A. It was reasoned that there were not enough cells to neutralize the higher doses of BoNT/A at the site where the toxins enter the circulation. 400 μl of blood were thus injected from the transplanted GPA-VNA/A chimeric mice into other recipients and found these mice were protected from 100 LD₅₀ BoNT/A challenge (FIG. 2B).

Several reports showed that the half-life of VHHs in vivo is short²² and unmodified anti-BoNT/A VNAs protect mice from BoNT/A challenge for less than a day post-administration 11. The half-life of an antibody can be prolonged, as shown in an in-human study that humanized monoclonal anti-BoNT/A antibodies had half-lives varying from 2.5 to 26.9 days depending on the dose of antibody administrated²³. Here, the half-life of the transfused engineered RBCs were analyzed in the circulation. To this end the RBCs were stained with a violet-trace dye prior to transfusion to monitor the total population of transfused RBCs and tracked cells expressing VNAs by GFP signal (from a GFP expression cassette in the lentivirus vector) (FIG. 2C). The half-life of control, GPA-VNA/A, and Kell-VNA/A RBCs were all approximately 14 days, similar to that of normal mouse RBCs. These mice were then challenged with 10 LD₅₀ BoNT/A at different times after transfusion and found that the antitoxin protective capacity of the engineered RBCs lasted up to 28 days (FIG. 2D). Taken together, the presently disclosed work demonstrated that covalently conjugating VNAs onto RBCs allows a dramatic extension of the circulatory half-life of the VNAs without compromising their neutralizing capacity.

To determine the fate of BoNT/A and the engineered RBCs in mice, violet-dyed wild type RBCs or GPA-VNA/A-expressing RBCs were incubated with catalytically inactive BoNT/A (ciBoNT/A) before transfusing the mixture into mice. The recipient mice were then bled at intervals and analyzed amounts of both free (FIG. 2E) and RBC-bound ciBoNT/A (FIG. 2F) in the serum. As shown in FIG. 2E, 1 hour after injection (“0 day” on the graph) there is very little free ciBoNT/A in the serum of mice transfused with GPA-VNA/A-expressing RBCs, compared to mice transfused with control RBCs. In contrast, ˜100 times more RBCs appear to have bound ciBoNT/A in mice transfused with GPA-VNA/A-expressing RBCs than in mice transfused with control blood based on the RBC surface S-tag signal (from the anti-ciBoNT/A probe) (FIG. 2F). RBC-bound ciBoNT/A is cleared from serum after 7 days (FIG. 2F), approximately concurrent with the GPA-VNA expressing RBCs (GFP+ cells) (FIG. 2G). Since the GPA-VNA/A expressing cells (GFP+ cells) were cleared significantly faster than the other RBCs (FIG. 2F), and similar to bound ciBoNT/A (FIG. 2G), a high percentage of GPA-VNA/A RBCs must have become bound to ciBoNT/A. It was hypothesized that binding of BoNT/A to GPA-VNA/A expressing RBCs enhances their degradation by macrophages or dendritic cells in the spleen or liver^{25, 26, 27}.

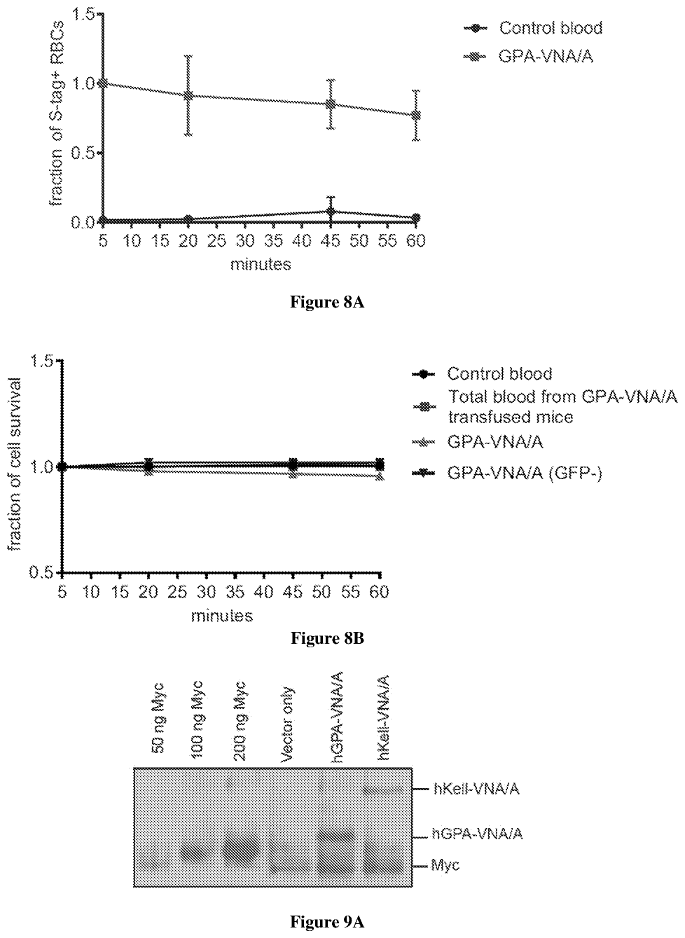

The same experiment was performed but bleeding intervals were taken within 1 hour after transfusion. Transfused GPA-VNA/A RBCs still bound ˜100 times more ciBoNT/A than control RBCs, despite the ˜20% loss of bound ciBoNT/A from the GPA-VNA/A RBCs at 1 hour compared to 5 minutes (FIG. 8). Undetectable numbers of transfused cells were lost between 5 minutes and 1 hour; therefore, using 1 hour as the “time 0” in the experiment in FIGS. 2E-2G is appropriate.

Multiple repeat antibody administration typically elicits an adverse immune response. The immune response in mice was therefore investigated after three injections of control RBCs, GPA-VNA/A RBCs, or equimolar amounts of recombinant VNA/A (FIG. 2H). ELISA was used to detect the relative abundance of anti-VNA/A antibodies in the in serum. At 1:333 and 1:111 serum dilutions, very little antibody was detected in mice receiving control or GPA-VNA/A RBCs, while large amounts of anti-VNA/A antibody were detected following injection of the VNA protein. As shown by the ELISA performed at a 1:37 serum dilution, the amount of anti-VNA produced against the VNA-expressing red cells is at least 10 fold less than that from VNA/A injected mice.

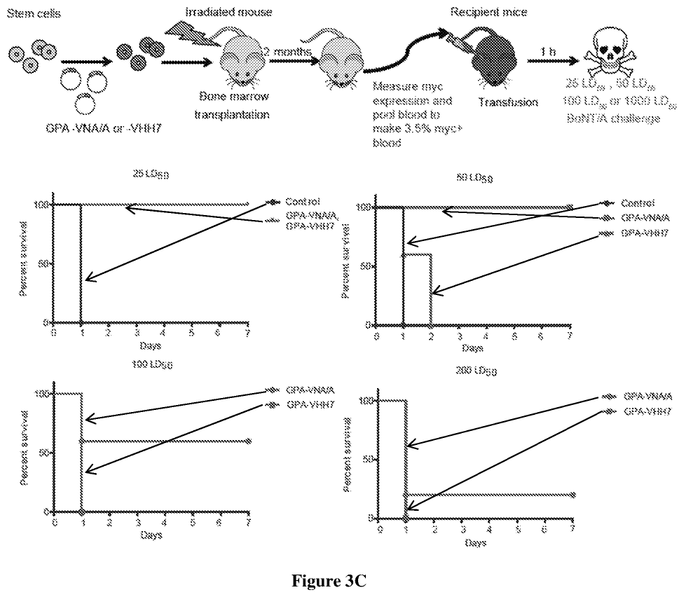

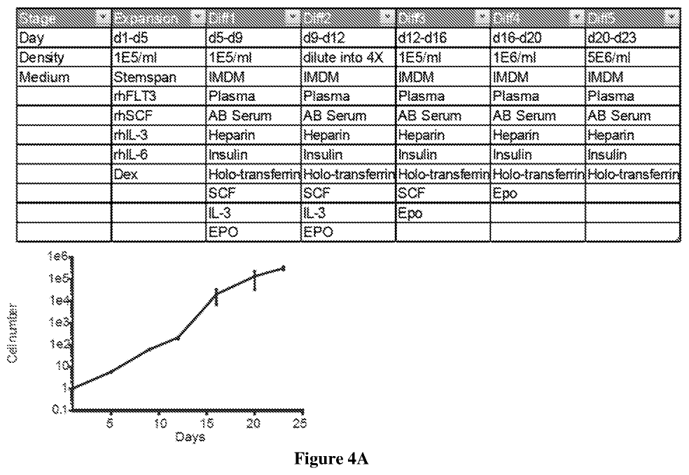

Given that murine RBCs expressing the GPA-VNA/A chimera are very potent, it was also examined whether monomeric VHHs can be used instead of bispecific VNAs. Thus, another plasmid (GPA-VHH7) was constructed to generate a chimeric protein fusing ciA-H7 VHH with GPA (FIG. 3A). Cultured mouse RBCs expressing GPA-VNA/A and GPA-VHH7 were then produced and shown to express similar levels of chimeric GPA and have similar BoNT/A neutralizing potency in neuronal cell-based assays (FIG. 3B). Mice transfused with an equivalent amount of cultured RBCs expressing either GPA-VNA/A or GPA-VHH7 were protected from 25 LD₅₀ of BoNT/A (FIG. 3C) while only the GPA-VNA/A mice were protected from 50 LD₅₀, and to a lesser extent at even higher doses. The results suggest there may be a small potency advantage in vivo when using RBCs displaying bispecific VNAs vs. monospecific VHHs.

Finally the possibility of producing engineered human RBCs from human CD34⁺ progenitor cells was explored. The current CD34⁺ cell culture protocol was modified and the cell number, differentiation markers, membrane proteins, and enucleation were measured at the end of each developmental stage. As shown in FIG. 4A (upper table), a six-stage culture system was developed, an improvement over the four-stage system used previously and the three-stage system used by other labs^{28,29}. The present modified system, mobilized bone marrow CD34⁺ stem/progenitor cells expanded more than 300,000-fold during the 23-day culture period (bottom panel). Differentiation was gradual and synchronous; as an example, c-kit expression was initially high and declined with differentiation whereas CD71 and CD235A expression increased. At the time when CD71 (transferrin receptor) expression began to decrease, cells began to enucleate; more than 90% of cells had undergone enucleation and become reticulocytes by the end of the culture (FIG. 4B). The enucleated cells are 7 am in diameter, similar to human reticulocytes³⁰, and contain 28.23±0.46 pg hemoglobin/cell, within the normal range for human RBCs (FIG. 4C)³¹. The survival of these in vitro differentiated human reticulocytes was also monitored in vivo and found to circulate for at least 7 days in macrophage-depleted nonobese diabetes/severe combined immunodeficiency (NOD/SCID) mice (FIG. 4D). About 94% of cultured reticulocytes survived in these mice at 1 day post-transfusion, and ˜36% cells at 3 days, comparable to recent published data³². Overall, the system was improved in terms of the number of differentiated, enucleated cells (93.53±2.84% enucleation). The expression of multiple surface proteins was compared at the end of each culture stage and it was shown that the expression pattern of these proteins is similar to that previously reported³³. As examples, GPA (CD235A) and Rhesus (CD240DCE) increase over time, and CD36, CD47, CD59, CD71, CD147, a4 integrin (CD49d), a5 integrin (CD49e), P31 integrin (CD29), and Kell show decreased expression during differentiation (FIG. 5A).

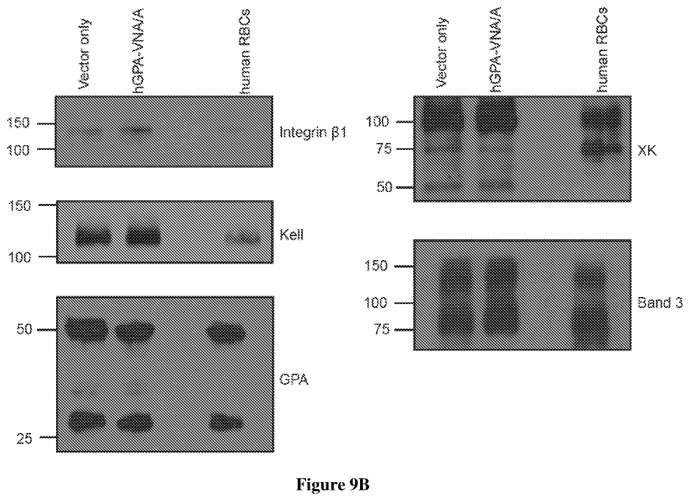

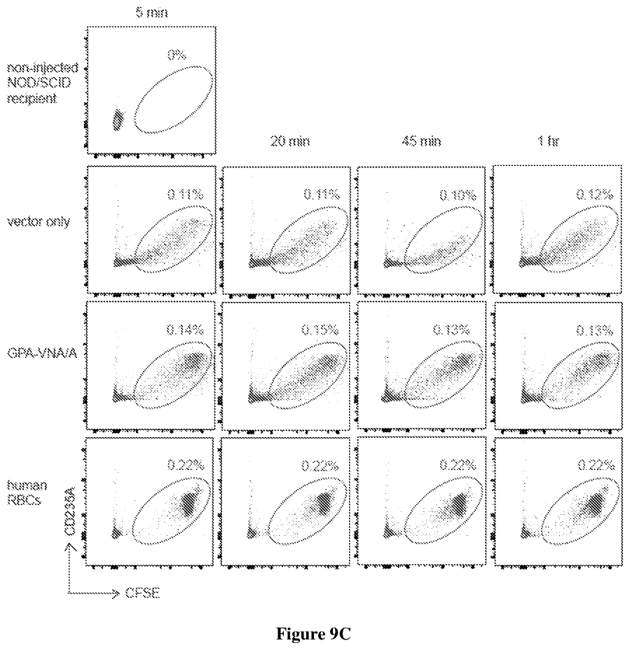

CD34⁺ stem/progenitor cells were then infected with lentiviral vectors that express the hGPA-VNA/A or hKell-VNA/A chimeric proteins depicted in FIG. 1A. Importantly, control and GPA-VNA/A expressing cells show similar expression patterns of multiple surface proteins, suggesting that expression of this chimeric protein does not significantly alter differentiation (FIG. 5A). Western blot analysis showed that the present six-stage cultured control or GPA-VNA/A expressing reticulocytes contain similar amounts of GPA, Kell, p31-integrin, band 3 and XK proteins, compared to human RBCs (FIG. 9B). Moreover, the morphology of vector control and GPA-VNA/A expressing CD34⁺ cells are similar to uninfected CD34⁺ cells at each stage, indicating that the cells differentiate properly (FIG. 5B, bottom.) However, infection of CD34⁺ cells leads to a reduced number of cells undergoing enucleation (to ˜70-80%; FIG. 5B, upper.) The growth rate is unperturbed following expression of GPA-VNA/A (FIG. 5C).

Enucleated cells express ˜500,000 copies of hGPA-VNA/A or 120,000 copies of Kell-VNA/A, as indicated by myc expression (FIG. 9A, see legend for calculation). The number of chimeric proteins in engineered human cells is lower than that in engineered mouse cells, which could result from varying numbers of viral particles infecting cells and from the use of different vectors with different promoters.

These engineered human RBCs were co-incubated with neurons in the presence of BoNT/A. Co-incubation with 1 million hGPA-VNA/A or hKell-VNA/A-expressing human RBCs partially inhibited BoNT/A activity and 5 million hGPA-VNA/A expressing human RBCs fully prevented SNAP25 cleavage (FIG. 5D). The in vitro cultured human reticulocytes survive following transfusion in NOD/SCID mice about as long as do normal human red cells (FIG. 4D) and this ability allowed us to examine the protective capacity of the engineered human RBCs in vivo. 15,000,000 hGPA-VNA/A-expressing human reticulocytes were transfused into NOD/SCID mice and challenged them with 10 LD₅₀ of BoNT/A. Consistent with the in vitro toxin neutralization results, these human reticulocytes provided protection against 10 LD₅₀ BoNT/A in all recipient mice (FIG. 5E). The different protection response is not due to different cell numbers remaining in the circulation of NOD/SCID mice, since the percentage of cells expressing vectors or GPA-VNA/A surviving in NOD/SCID mice is similar from 5 minutes-post-transfusion to 1 hour (FIG. 9C).

### Discussion

Here the production of genetically engineered RBCs that carry cargoes of therapeutic value is described; in this case, VHHs that target a bacterial toxin, and that have long circulatory half-life. First, it was demonstrated that VHHs fused to a GPA or Kell protein can be functionally expressed on the surface of normal enucleated mouse RBCs and human reticulocytes. These modifications of RBCs do not materially affect their biogenesis; as judged by surface protein expression, proliferation, cell size, and hemoglobin content. Second, these chimeric VHHs retain their potency in in vitro BoNT/A neutralization assays and in vivo toxin challenges. Notably, mice transfused with red blood cells expressing the GPA-VNA/A chimera, such that the engineered cells comprise less than 1% of the total mouse RBCs, survive a BoNT/A challenge of 10 LD₅₀ BoNT/A for up to 28 days. Third, a modified culture system and genetically modified human CD34⁺ stem/progenitor red blood cells was used to produce enucleated human reticulocytes expressing the hGPA-VNA/A chimera, and showed that transfusion of these cells into immune-compromised mice rendered them resistant to challenge by a 10 LD₅₀ dose of BoNT/A. The RBC engineering method should have widespread applications for prolonging half-life in the circulation of any enzyme or anti-toxin used for prophylactic or therapeutic treatments.

Sortase A-mediated modification (sortagging) was previously reported to attach cargoes covalently to RBCs³⁴. Sortase A recognizes an LPXTG motif and cleaves the peptide bond between the threonine and glycine residues to yield a thioester acyl-enzyme intermediate. A nucleophile that contains suitably exposed N-terminal glycines, (G), can resolve this intermediate, covalently linking the two motifs via a peptide bond³⁵. Despite the flexibility of cargo selection provided by sortase A-mediated cargo delivery, human and mouse red cells contain only ˜3,000 to 8,000 surface proteins with N-terminal (G)ₙ motifs, which limits the cargo loading numbers. The genetic engineering method detailed in this report provides a way to bypass this challenge, permitting greatly increased cargo capacity.

Compared with other RBC engineering methods, the present methods are better-suited for long-term, persistent delivery of cargo. For instance, RBC membrane-coating techniques produce RBC-membrane-camouflaged polymeric nanoparticles by deriving membrane vesicles from RBCs and fusing these vesicles with nanoparticles. This protocol enables the cargo to last ˜50 hr. in circulation³⁶, while the genetically engineered mouse RBCs circulate in the bloodstream for ˜28 days. Covalent attachment of cargo onto RBCs not only prolongs in vivo retention times of chimeric proteins but also avoids their rapid clearance⁸. It was observed that the engineered RBCs that have bound the antigen (toxin in the present experiments) are cleared slightly faster than are unperturbed engineered RBCs. It is not clear whether this half-life difference is due to the large size of the bound BoNT/A (150 kDa) or the binding of antigen itself; it will be interesting to attach other VHHs, whose target antigens differ in size and other properties, and determine the effects on RBC clearance. Another possibility is that these toxin carrying RBCs are somehow seen by the cells of the reticuloendothelial system as damaged RBCs and cleared by macrophages or dendritic cells.

It was shown that a single VHH (GPA-VHH7) is also able to neutralize BoNT/A. Since each VHH comprises a single immunoglobulin domain stabilized by one or two intramolecular disulfide bonds that fold independently^{37, 38}, it is likely possible to engineer GPA or Kell chimeras that contain three or more VHH domains and express these on the same RBCs. In this way one could engineer RBCs that bind multiple foreign toxins or viruses, and thus offer long-term prophylactic protection against multiple pathogens.

Systemic administration of foreign proteins carries significant risk of inducing a strong antibody response. This is especially the case for neutralizing antibodies derived from mice³⁹. Such agents usually require humanization at the expense of reducing affinity and specificity. In worst-case scenarios, even with humanization, they may prove too immunogenic to be administered more than a handful of times⁴⁰. Nanobodies derived from camelids, on the other hand, pose less of an immunogenic risk⁴¹. Here it was assessed whether long-term protection against BoNT/A comes with a side effect immune response by carrying out repeated transfusions of GPA-VNA/A RBCs into a cohort of C57BL/6J recipients at day 1, day 21, and day 28. In parallel, unmodified RBCs or equimolar amounts of recombinant VNA/A proteins were also transfused with the same schedule. Multiple transfusions of GPA-VNA/A RBCs were found to elicit an insignificant antibody response against intact VNA/A proteins, comparable to mice receiving unmodified RBCs, whereas mice that received VNA/A proteins developed a much higher titer of antibody response against intact VNA/A proteins (FIG. 2H). This result implies that the RBC vehicle, possibly by masking the VNA as the RBC's own proteins, can further minimize immune response against the covalently attached VNA. Recently it was shown that red cells with several different peptides covalently linked to their surface do not induce an immune response to these peptides after transfusion into a naïve mouse. Rather, they induce tolerance to these peptide antigens. Therefore, GPA-VNA/A RBCs and other engineered RBCs generated using similar methods can not only provide long-term protection, but may also permit multiple administrations without provoking the immune system.

Based on previously reported culture systems using mobilized human bone marrow CD34⁺ cells to produce red blood cells, which yield a lower number of cells²⁸ and a lower percentage of enucleated cells^{33,42,32} the six-stage culture system offers enhanced enucleation. The cell seeding density was increased and the erythropoietin level was reduced during the last stage, suggesting that these two factors positively affect reticulocyte production. Despite the higher percentage of enucleated cells, the maturation status of the reticulocytes has not been investigated and it cannot be concluded that these cells are fully mature red cells. Reduced enucleation is observed in cells infected with lentiviruses, raising the safety concern of transmission of genetic material. While one possible solution is to filter nuclei and nucleated cells by leucocyte filtration as performed in other studies³¹, adjusting the transduction protocol should be another possible method to reduce influence on culture efficiency. On the other hand, there are several clinical trials of cellular therapy using lentivirus transduced cells that show great safety^{43 44}. Improving vector designs to enable safe delivery should be a consideration when engineering human RBCs in vitro.

Previous reports showed that the enucleated reticulocytes produced in culture mature normally into biconcave mature RBCs following transfusion into NOD/SCID mice, eliminating the necessity of further in vitro maturation⁴⁵. More importantly, Giarratana et al. have shown that the half-life of in vitro cultured RBCs in the human body is ˜26 days⁴⁵. Accordingly, engineered human reticulocytes carrying BoNT/A VHHs, which are produced in vitro, should provide effective protection from lethal BoNT/A challenges in humans.

### Materials and Methods

### Plasmids and Generation of Recombinant Retroviruses and Lentiviruses

MSCV-based retroviruses were produced and used to infect erythroid progenitors following a previously described protocol,⁴⁶ and lentivirus vectors used to infect human cells were also described previously⁴⁷. The ciA-H7 and ciA-B5 monomer sequences, and the bispecific H7/B5 heterodimer sequence, were previously reported³ and the D10 and G10 sequences are unpublished (but are available from Dr. Shoemaker). Human Glycophorin A and Kell cDNA sequences were obtained from the National Center for Biotechnology Information (NCBI), reference sequences NM_002099.6 and NM_000420.2, respectively. These sequences were used for synthesizing DNA fragments; these DNA fragments were cloned into XhoI-cut XZ201 or HMD plasmids using a Gibson assembly kit. HMD vector uses a HIV/murine stem cell virus hybrid long terminal repeat as the promoter. The XZ201 plasmid was used for retrovirus production and the HMD plasmid was used for lentivirus production. Both contain GFP inserted after an IRES sequence which drives GFP expression.

293T cells were cultured in Dulbecco's Modified Eagle Medium (DMEM) with 10% FBS in a humidified 5% CO₂ atmosphere at 37° C. XZ201 based plasmids and pCLECO or HMD based plasmids and packaging vectors, VSV-G and pD8.9, were incubated with medium and Fugene 6 according to the Promega protocol. The mixture medium was changed after 6 h; retrovirus and lentivirus were collected after 24 h and 72 h for transfection, respectively.

### Flow Cytometry Analyses and Antibodies

All flow cytometry data were acquired on a FACS Fortessa flow cytometer (BD Biosciences) and analyzed using Flowjo software (Tree Star). All stainings were carried out in FACS buffer (2 mM EDTA and 5% FBS in PBS) for 40 minutes at room temperature unless otherwise described. Samples were washed twice with FACS buffer prior to flow analyses. The following are the antibodies used: anti-human CD235A-APC (eBioscience, 17-9987042), anti-human CD71-FITC (eBioscience, 11-0719-42), anti-human CD71-PeCy7 (Affymetrix, 25-0719-42), anti-human CD117-PeCy7 (eBioscience, 25-1178-42), anti-human CD117-BV605 (Biolegend, 313217), anti-human CD49e-APC (BioLegend, 328012), anti-human CD29-PerCP-eFluor® 710 (Affymetrix, 46-0299-41), anti-human CD49d-PE (Affymetrix, 12-0499-42), anti-human CD240DCE-APC (Miltenyi Biotec, 130-104-818), anti-human CD238-APC (Miltenyi Biotec, 130-104-951), anti-human CD47-PerCP-eFluor® 710 (Affymetrix, 46-0479-42), anti-human CD147-PE (Affymetrix, 12-1472-42), anti-human CD59-APC (Affymetrix, 17-0596-42), anti-myc tag-PE (Cell Signaling Technology, 3739), anti-mouse Ter119-APC (eBioscience, 17-5921-83), and anti-mouse CD71-PE (Affymetrix, 12-0711-83). Hoechst 33342 (Life Technologies, H1399) was used to visualize nuclei.

### Human CD34⁺ Cell Culture

Granulocyte-colony stimulating factor (G-CSF)-mobilized CD34⁺ peripheral blood stem cells (purchased from the Fred Hutchinson Cancer Center) were thawed according to the vendor's protocol. Cells were then placed in expansion medium containing 100 ng/ml rhFlt-3 ligand, 100 ng/ml rhSCF, 20 ng/ml rhIL6, 20 ng/ml rhIL3 and 100 nM Dexamethasone in Stemspan II medium for 5 d at a density of 100,000 cells/ml. The cells were then cultured in differentiation 1-2 medium (2% human blood plasma, 3% human serum, 3 U/ml heparin, 10 ug/ml insulin, 200 ug/ml holo-transferrin, 10 ng/ml rhSCF, 1 ng/ml IL-3 and 3 U/ml Epo in IMDM) at a density of 100,000 cells/ml for 4d and at 200,000 cells/ml for 3 d. The medium was switched to Differentiation 3 medium (2% human blood plasma, 3% human serum, 3 U/ml heparin, 10 ug/ml insulin, 200 ug/ml holo-transferrin, 10 ng/ml rhSCF and 1 U/ml Epo in IMDM) and the cell density was maintained at 100,000 cells/ml. After 4 days, the cells were cultured at a density of 1,000,000 cells/ml in Differentiation 4 medium (2% human blood plasma, 3% human serum, 3 U/ml heparin, 10 ug/ml insulin, 500 ug/ml holo-transferrin and 0.1 U/ml Epo in IMDM) for an additional 4 days. For culture stage 5, the medium was replaced by Differentiation 5 medium (2% human blood plasma, 3% human serum, 3 U/ml heparin, 10 ug/ml insulin, 500 ug/ml holo-transferrin in IMDM) and the density was increased to 5,000,000 cells/ml. Enucleated RBCs were ready to be used after 3 days.

### Hemoglobin Content Measurement and Histology Stain of CD34⁺ Cells

Cells were stained on slides with May-Griinwald-Giemsa and diaminobenzidine hydrochloride reagents (Sigma-Aldrich GS-500 and D-9015) for morphological analyses. Hemoglobin content was measured at 540 nm wavelength light after incubating 1 million cells with Drabkin's reagent (RICCA chemical company, 2660-16). Human hemoglobin (Sigma-Aldrich H7379) was used for the standard curve to calculate hemoglobin amounts from the O.D.450 nm value.

### Antibodies for Western Blotting to Detect Human CD34⁺ Cell Proteins

Anti-XK (Thermo Fisher Scientific, PIPA540782), anti-EPB41 (Abcam, ab54597), anti-MPP1 (Abcam, ab96255), anti-CD47 (Abcam, ab3283), anti-band 3 (Santa Cruz Biotechnology, sc-133190), anti-Integrin P31 (Santa Cruz Biotechnology, sc-18887), anti-Glycophorin C (Santa Cruz Biotechnology, sc-59183), anti-Glycophorin A (Santa Cruz Biotechnology, sc-59182), anti-Kell (Santa Cruz Biotechnology, sc-271070)

Isolation of Erythroid Progenitors from Murine Fetal Liver Cells

Enriched erythroid progenitors were purified from E14.5 C57BL/6J mouse embryos, and cultured in vitro for erythroid differentiation following a protocol described in detail previously³⁴. Briefly, pregnant C57BL/6J mice at embryonic day 14.5 were sacrificed by CO₂ asphyxiation and the embryos collected. The fetal livers were isolated and suspended in PBS with 2% FBS and 100 μM EDTA. Mature RBCs in the cell suspension were lysed by incubation for 10 min with an ammonium chloride solution (Stemcell). Following the manufacturer's protocol, lineage negative cells were obtained after magnetic depletion of lineage positive cells using the BD Pharmingen Biotin MouseLineage Panel (559971; BD Biosciences) and BD Streptavidin Particles Plus-DM (557812; BD Biosciences). These lineage negative fetal liver cells were enriched more than 90% for erythroid progenitors.

### Viral Infection and Culture of Murine Erythroid Progenitors

MSCV-based retroviruses were produced and used to infect erythroid progenitors following a previously described protocol⁴⁶. Briefly, after isolation, lineage negative fetal liver cells were plated in 24-well plates at 100,000 cells per well, covered by 1 ml virus containing supernatant, and centrifuged at 2000 RPM for 90 min at 30° C. After this spin-infection, the virus supernatant was replaced with erythroid maintenance medium (StemSpan-SFEM; StemCell Technologies) supplemented with 100 ng/mL recombinant mouse stem cell factor (SCF) (R&D Systems), 40 ng/mL recombinant mouse IGF1 (R&D Systems), 100 nM dexamethasone (Sigma), and 2 u/mL erythropoietin (Amgen) and cultured at 37° C. GFP+ cells were sorted by flow cytometry after 16 h and cultured for another 48 h in erythroid differentiation medium (Iscove modified Dulbecco's medium containing 15% (vol/vol) FBS (Stemcell), 1% detoxified BSA (Stemcell), 500 μg/mL holo-transferrin (Sigma-Aldrich), 0.5 U/mL Epoetin (Epo; Amgen), 10 μg/mL recombinant human insulin (Sigma-Aldrich), and 2 mM L-glutamine (Invitrogen)) at 37° C.

### Transplantation of Mouse Fetal Liver Cells

C57BL/6J (Jackson Laboratory) or CD-1 (Charles River) mice were subjected to lethal irradiation of 1,050 rad carried out in a Gammacell 40 irradiator chamber (Nordion International Inc.) one day before transplantation. 1,500,000 virally-infected murine erythroid progenitors from mouse fetal liver cultured in erythroid maintenance medium, produced as described in the previous paragraph, were harvested and resuspended in 100 μl PBS³⁴. These cells were then injected retro-orbitally into an irradiated mouse. Mice were bled for examining chimeric protein expression and further analysis 5 weeks after transplantation. Mice were bled at indicated time points for performing Complete blood count analysis on a SIEMENS ADVIA 2120i machine.

### Transfusion and In Vivo Survival of GPA-VNA RBCs

200 μl blood from transplanted mice containing RBCs expressing the GPA-VNA chimera was collected into heparinized tubes (Fisher Scientific, 365965). RBCs were washed twice in PBS and resuspended in PBS. Labeling was then carried out with 5 μM CellTrace Violet dye for 20 minutes at room temperature (Life Technologies). 10% FBS in PBS was then added to RBCs for quenching the staining reaction. Violet-labeled RBCs were washed twice with PBS and resuspended in 200 μl sterile PBS for intravenous injection into recipient mice. An equal volume of unmodified RBCs, similarly stained, served as a control.

A drop of blood, ˜20 μl, was collected into heparinized tubes by retro-orbital bleeding 1 hour after transfusion at day 0 and every 3-4 days for 1 month as indicated in the text. These blood samples were washed once with FACS buffer and then subjected to staining with anti-Ter119-APC and anti-myc tag-PE for 30 min on ice. Samples were washed twice with FACS buffer prior to analyses on a FACS Fortessa flow cytometer for violet fluorescence and for GFP, Ter119, and myc signals.

ciBoNT/a Flow Cytometry and ELISA Assays for Toxin Binding to RBCs (FIGS. 2F-2G) and Quantifying Toxin in Serum (FIG. 2E)

200 μl blood from transplanted mice that contain RBCs expressing the GPA-VNA chimera was collected into heparinized tubes and stained with CellTrace Violet dye as described above. After resuspending the violet-stained RBCs in 200 μl PBS, RBCs were further incubated with 1 μg catalytically inactive recombinant BoNT/A (ciBoNT/A) on ice for 40 minutes prior to intravenous injection into recipient mice. An equal volume of unmodified RBCs, also stained and incubated with ciBoNT/A, served as a control. One drop of blood was collected into heparinized tubes by retro-orbital bleeding 1 hour after transfusion (termed day 0) and every 3-4 days for 14 days as indicated in the text and figure legend. Samples were washed once with FACS buffer and then subjected to staining with anti-myc tag-PE to detect surface expressed VNAs and anti-S tag-APC (VWR, 10065-802) to indirectly detect ciBoNTA by incubation with the bispecific BoNT/A-binding heterodimer, ciA-F12/D12³ which carries the S-tag. Samples were washed twice with FACS buffer prior to analyses on a FACS Fortessa flow cytometer for violet fluorescence, GFP, and S-tag (indicating toxin that is bound to the RBC surface) signals.

In a parallel experiment to measure ciBoNT/A in serum of transfused mice (FIG. 2E), 200 μl blood from transplanted mice that contain RBCs expressing the GPA-VNA chimera was also collected into heparinized tubes. RBCs were washed twice in PBS and resuspended in PBS to a volume of 200 μl. As above, these RBCs were incubated with 2 ng ciBoNT/A on ice for 40 minutes prior to intravenous injection into recipient mice. The recipient mice were then bled 1 hour after transfusion (day 0) and every week for 1 month as indicated in the text. 100 μl blood was collected into a serum separator tube (Fisher Scientific, 02675185) and blood samples were allowed to clot at room temperature for 15-30 minutes. Serum and clots were then separated by centrifugation at 10,000 g for 10 minutes at 4° C.

ELISA plates coated with polyclonal anti-BoNT/A (Metabiologics, Inc) were blocked with blocking buffer (0.05% Tween20+2% BSA in PBS) for 2 hours at room temperature prior to addition of 10 μl serum samples in 90 μl PBS; the incubation period was 3.5 hours at room temperature, followed by 4 washes with PBS. Plates were then incubated with VHH ciA-D12³ against BoNT/A, which carries an E-tag, and anti-E tag-HRP (Ethyl Laboratories, A190-133P), both at 1:1000 dilutions in blocking buffer for 1 hour. Plates were then washed again 4 times with PBS and developed with 3,3′,5,5′-Tetramethylbenzidine (TMB) liquid substrate reagent (Sigma). Reactions were stopped with 1N HCl and read at 450-nm absorbance. As a quantification standard, serially diluted 1 μg catalytically inactive recombinant BoNT/A was used.

### ELISA for Detecting Antibody Response Against VNA/as

Freshly bled 200p1 GPA-VNA/A RBCs and WT C57BL/6J RBCs were washed once with DMEM medium, twice in PBS, and resuspended in sterile PBS to make 200 μl final volume for intravenous injection into C57BL/6J recipient mice. A separate cohort of C57BL/6J recipient mice was also transfused with equimolar amounts of recombinant VNA/As in 200 μl PBS. Three and four weeks later, 2^{nd} and 3^{rd} transfusions were carried out in a similar fashion, using the same cohort of recipient mice. Serum samples were collected from these mice 5 days after the last transfusion. 96-well plates were coated with 10 μg/ml recombinant VNA/A in PBS overnight at 4° C. and blocked in blocking buffer (10% heat inactivated FBS in PBS) prior to addition of 5 μl serum samples in 180 μl blocking buffer. Incubation with test serum was for 3 hours at room temperature. Plates were washed 4 times with PBS, incubated with goat anti-mouse IgG-HRP (SouthernBiotech) at 1:10,000 in blocking buffer for 1 hour, and developed with 3,3′,5,5′-Tetramethylbenzidine (TMB) liquid substrate reagent (Sigma). The reaction was stopped with 1N HCl and absorbance was read at 450 nm.

### Neuron Culture and BoNT/a Neutralization Assay

A previously described protocol was followed³. In brief, rat (Sprague Dawley strain) cortical neurons were prepared from E18-19 embryos. Dissected cortices were dissociated with papain following the manufacturer's instructions (Worthington Biochemical, NJ) and cells were plated on poly-D-lysine coated 24-well plates at 250,000 cells per well in 1 ml Neurobasal medium (Thermo Fisher, Cat. No. 21103-049) supplemented with 2% B27 (Thermo Fisher, Cat. No. 17504-001) and Glutamax (Thermo Fisher, Cat. No. 35050-061). Experiments were carried out using DIV (days in vitro) 11 neurons.

RBCs were pelleted (1,500 rpm, 5 min) and re-suspended in conditioned neuron culture medium taken from neuron culture plates (0.5 ml per well), and 20 pM BoNT/A was added. The mixture of RBCs and BoNT/A was incubated at 37° C. for 30 mins and then added to the original well containing the cultured neurons. Neurons were cultured further for 9 hours. Neuron lysates were then collected in 100 μl RIPA buffer (50 mM Tris, 1% NP40, 150 mM NaCl, 0.1% SDS, plus a protease inhibitor cocktail (Millipore)). Lysates were centrifuged for 10 min at 12000 rpm at 4° C. Supernatants were subjected to SDS-PAGE and immunoblot analysis using a SNAP25 antibody⁴⁸ and the enhanced chemiluminescence (ECL) method⁴⁹.

### Mouse Survival Assay

The present mouse studies were conducted under animal protocols approved by the Division of Comparative Medicine at MIT or the Tufts University IACUC. Eight-week-old female CD-1, C57BL/6J or NOD/SCID (Jackson Laboratory) mice were weighed before toxin injection and blood transfusion. The average weight of the mice was used to calculate the lethal dose of BoNT/A toxin (Metabiologics, Inc); the LD₅₀ of BoNT/A that was employed for C57BL/6J was 1.5 μg/g, which was measured under the animal protocol. The LD₅₀ of BoNT/A for CD-1 mice is 1.86 μg/g. The LD₅₀ of BoNT/B is 1.58 μg/g. When co-administered with toxin, wild type or engineered RBCs were transfused in a 200 μl PBS solution retro-orbitally (C57BL/6J mice) or intravenously (CD-1 mice) 30 min-2 hour prior to administration of BoNT/A. Mice were observed 2 to 6 times/day for 7 days. Mice with symptoms such as difficulty moving, open-mouth breathing, and wasp like narrow waist were euthanized and scored as moribund.

### Transfusion and In Vivo Survival of In Vitro-Differentiated Human Reticulocytes

At day 23, in vitro-differentiated human reticulocytes were collected, counted, washed once in PBS, and resuspended in PBS. Labeling of these human reticulocytes was carried out using 5 μM carboxyfluorescein succinimidyl ester (CFSE) according to the manufacturer's instructions (Life Technologies). 10% FBS in PBS was then added to the reticulocytes for quenching the staining reaction. CFSE-labeled reticulocytes were then washed twice with PBS and resuspended in 200 μl sterile PBS for intravenous injection into NOD/SCID mice at 250,000,000 reticulocytes per mouse. These recipient mice had been intraperitoneally injected with 100 μl and 50 μl clodronate liposomes at three days and one day prior to transfusion, respectively.

A drop of blood, ˜20 μl, was collected into heparinized tubes by retro-orbital bleeding for 1 week starting at 1 hour after transfusion at day 0 and at 5 more time points as indicated in the text. These blood samples were washed once with FACS buffer and then subjected to staining with anti-CD235A-APC for 30 min on ice. Samples were washed twice with FACS buffer prior to analyses on a FACS Fortessa flow cytometer for CFSE and CD235A signals.

### REFERENCES

1. Hamers-Casterman C, et al. Naturally occurring antibodies devoid of light chains. Nature 363, 446-448 (1993).
2. Muyldermans S. Nanobodies: natural single-domain antibodies. Annual review of biochemistry 82, 775-797 (2013).
3. Mukherjee J, et al. A novel strategy for development of recombinant antitoxin therapeutics tested in a mouse botulism model. PLoS One 7, e29941 (2012).
4. Tremblay J M, et al. A single VHH-based toxin-neutralizing agent and an effector antibody protect mice against challenge with Shiga toxins 1 and 2. Infect Immun 81, 4592-4603 (2013).
5. Yang Z, et al. A novel multivalent, single-domain antibody targeting TcdA and TcdB prevents fulminant Clostridium difficile infection in mice. J Infect Dis 210, 964-972 (2014).
6. Moayeri M, et al. A heterodimer of a VHH (variable domains of camelid heavy chain-only) antibody that inhibits anthrax toxin cell binding linked to a VHH antibody that blocks oligomer formation is highly protective in an anthrax spore challenge model. J Biol Chem 290, 6584-6595 (2015).
7. Herrera C, Tremblay J M, Shoemaker C B, Mantis N J. Mechanisms of Ricin Toxin Neutralization Revealed through Engineered Homodimeric and Heterodimeric Camelid Antibodies. J Biol Chem 290, 27880-27889 (2015).
8. Chakravarty R, Goel S, Cai W. Nanobody: the “magic bullet” for molecular imaging?Theranostics 4, 386-398 (2014).
9. Vaidyanathan V V, et al. Proteolysis of SNAP-25 isoforms by botulinum neurotoxin types A, C, and E: domains and amino acid residues controlling the formation of enzyme-substrate complexes and cleavage. J Neurochem 72, 327-337 (1999).
10. Thanongsaksrikul J, Chaicumpa W. Botulinum Neurotoxins and Botulism: A Novel Therapeutic Approach. Toxins 3, 469-488 (2011).
11. Mukherjee J, et al. Prolonged prophylactic protection from botulism with a single adenovirus treatment promoting serum expression of a VHH-based antitoxin protein. PLoS One 9, e106422 (2014).
12. Adekar S P, et al. Enhanced neutralization potency of botulinum neurotoxin antibodies using a red blood cell-targeting fusion protein. PLoS One 6, e17491 (2011).
13. Byun H M, et al. Erythrocyte ghost-mediated gene delivery for prolonged and blood-targeted expression. Gene Ther 11, 492-496 (2004).
14. Muzykantov V R. Drug delivery by red blood cells: vascular carriers designed by mother nature. Expert Opin Drug Deliv 7, 403-427 (2010).
15. Kontos S, Kourtis I C, Dane K Y, Hubbell J A. Engineering antigens for in situ erythrocyte binding induces T-cell deletion. Proc Natl Acad Sci USA 110, E60-68 (2013).
16. Ji P, Murata-Hori M, Lodish H F. Formation of mammalian erythrocytes: chromatin condensation and enucleation. Trends Cell Biol 21, 409-415 (2011).
17. Villa C H, Anselmo A C, Mitragotri S, Muzykantov V. Red blood cells: Supercarriers for drugs, biologicals, and nanoparticles and inspiration for advanced delivery systems. Adv Drug Deliv Rev, (2016).
18. Cowley H, Wojda U, Cipolone K M, Procter J L, Stroncek D F, Miller J L. Biotinylation modifies red cell antigens. Transfusion 39, 163-168 (1999).
19. Muzykantov V R, Murciano J C. Attachment of antibody to biotinylated red blood cells: immuno-red blood cells display high affinity to immobilized antigen and normal biodistribution in rats. Biotechnol Appl Biochem 24 (Pt 1), 41-45 (1996).
20. Murciano J C, Medinilla S, Eslin D, Atochina E, Cines D B, Muzykantov V R. Prophylactic fibrinolysis through selective dissolution of nascent clots by tPA-carrying erythrocytes. Nat Biotechnol 21, 891-896 (2003).
21. Intentionally left blank.
22. Harmsen M M, De Haard H J. Properties, production, and applications of camelid single-domain antibody fragments. Appl Microbiol Biotechnol 77, 13-22 (2007).
23. Nayak S U, et al. Safety and pharmacokinetics of XOMA 3AB, a novel mixture of three monoclonal antibodies against botulinum toxin A. Antimicrob Agents Chemother 58, 5047-5053 (2014).
24. Intentionally left blank.
25. Piomelli S, Seaman C. Mechanism of red blood cell aging: relationship of cell density and cell age. Am J Hematol 42, 46-52 (1993).
26. Franco R S. The measurement and importance of red cell survival. Am J Hematol 84, 109-114 (2009).
27. Bosman G J. Survival of red blood cells after transfusion: processes and consequences. Front Physiol 4, 376 (2013).
28. Lee H Y, et al. PPAR-alpha and glucocorticoid receptor synergize to promote erythroid progenitor self-renewal. Nature 522, 474-477 (2015).
29. Douay L, Giarratana M C. Ex vivo generation of human red blood cells: a new advance in stem cell engineering. Methods Mol Biol 482, 127-140 (2009).
30. Malleret B, et al. Significant biochemical, biophysical and metabolic diversity in circulating human cord blood reticulocytes. PLoS One 8, e76062 (2013).
31. Griffiths R E, et al. Maturing reticulocytes internalize plasma membrane in glycophorin A-containing vesicles that fuse with autophagosomes before exocytosis. Blood 119, 6296-6306 (2012).
32. Kupzig S, Parsons S F, Curnow E, Anstee D J, Blair A. Superior survival of ex vivo cultured human reticulocytes following transfusion into mice. Haematologica 102, 476-483 (2017).
33. Hu J, et al. Isolation and functional characterization of human erythroblasts at distinct stages: implications for understanding of normal and disordered erythropoiesis in vivo. Blood 121, 3246-3253 (2013).
34. Shi J H, et al. Engineered red blood cells as carriers for systemic delivery of a wide array of functional probes. P Natl Acad Sci USA 111, 10131-10136 (2014).
35. Ton-That H, Mazmanian S K, Faull K F, Schneewind O. Anchoring of surface proteins to the cell wall of Staphylococcus aureus. Sortase catalyzed in vitro transpeptidation reaction using LPXTG peptide and NH(2)-Gly(3) substrates. J Biol Chem 275, 9876-9881 (2000).
36. Hu C M J, Zhang L, Aryal S, Cheung C, Fang R H, Zhang L F. Erythrocyte membrane-camouflaged polymeric nanoparticles as a biomimetic delivery platform. P Natl Acad Sci USA 108, 10980-10985 (2011).
37. Sheoran A S, et al. Adenovirus vector expressing Stx1/Stx2-neutralizing agent protects piglets infected with Escherichia coli O157:H7 against fatal systemic intoxication. Infect Immun 83, 286-291 (2015).
38. Schmidt D J, et al. A Tetraspecific VHH-based Neutralizing Antibody Modifies Disease Outcome in Three Animal Models of Clostridium difficile Infection. Clin Vaccine Immunol, (2016).
39. Baker M P, Reynolds H M, Lumicisi B, Bryson C J. Immunogenicity of protein therapeutics: The key causes, consequences and challenges. Self Nonself 1, 314-322 (2010).
40. Harding F A, Stickler M M, Razo J, DuBridge R B. The immunogenicity of humanized and fully human antibodies: residual immunogenicity resides in the CDR regions. MAbs 2, 256-265 (2010).
41. Vincke C, Loris R, Saerens D, Martinez-Rodriguez S, Muyldermans S, Conrath K. General strategy to humanize a camelid single-domain antibody and identification of a universal humanized nanobody scaffold. J Biol Chem 284, 3273-3284 (2009).
42. Giarratana M C, et al. Ex vivo generation of fully mature human red blood cells from hematopoietic stem cells. Nat Biotechnol 23, 69-74 (2005).
43. McGarrity G J, et al. Patient monitoring and follow-up in lentiviral clinical trials. J Gene Med 15, 78-82 (2013).
44. Naldini L. Gene therapy returns to centre stage. Nature 526, 351-360 (2015).
45. Giarratana M C, et al. Proof of principle for transfusion of in vitro-generated red blood cells. Blood 118, 5071-5079 (2011).
46. Zhang X, Ren R. Bcr-Abl efficiently induces a myeloproliferative disease and production of excess interleukin-3 and granulocyte-macrophage colony-stimulating factor in mice: a novel model for chronic myelogenous leukemia. Blood 92, 3829-3840 (1998).
47. Ludwig L S, et al. Altered translation of GATA1 in Diamond-Blackfan anemia. Nature medicine 20, 748-753 (2014).
48. Peng L, et al. Cytotoxicity of botulinum neurotoxins reveals a direct role of syntaxin 1 and SNAP-25 in neuron survival. Nat Commun 4, 1472 (2013).
49. Yien Y Y, et al. TMEM14C is required for erythroid mitochondrial heme metabolism. The Journal of clinical investigation 124, 4294-4304 (2014).

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A method for producing human red blood cells or enucleated precursors thereof, the method comprising:
(i) providing a population of human CD34⁺ progenitor cells;
(ii) expanding the population of human CD34⁺ progenitor cells in a expansion medium for 1 to 6 days, wherein the expansion medium comprises Flt-3 ligand, stem cell factor (SCF), interleukin 3 (IL-3), and interleukin 6 (IL-6);
(iii) differentiating the population of human CD34⁺ progenitor cells expanded in (ii) in a first differentiation medium for 2 to 6 days, wherein the first differentiation medium comprises SCF, IL-3, insulin, erythropoietin (EPO), and holo-transferrin;
(iv) differentiating the population of human CD34⁺ progenitor cells from (iii) in the first differentiation medium for 1 to 5 days,
(v) differentiating the population of human CD34⁺ progenitor cells from (iv) in a second differentiation medium for 2 to 6 days, wherein the second differentiation medium comprises SCF, insulin, EPO, and holo-transferrin;
(vi) differentiating the population of human CD34⁺ progenitor cells from (v) in a third differentiation medium for 2 to 6 days, wherein the third differentiation medium comprises insulin, EPO, and holo-transferrin; and
(vii) differentiating the population of human CD34⁺ progenitor cells from (vi) in a fourth differentiation medium for 1 to 4 days, wherein the fourth differentiation medium comprises insulin, and holo-transferrin, thereby producing human red blood cells or enucleated precursors thereof.

2. The method of claim 1, wherein the expansion medium comprises Flt-3 ligand at a concentration of 50 ng/ml to 200 ng/ml, SCF at a concentration of 50 ng/ml to 200 ng/ml, IL-3 at a concentration of 10 ng/ml to 40 ng/ml; and IL-6 at a concentration of 10 ng/ml to 40 ng/ml.

3. The method of claim 1 or 2, wherein the expansion medium further comprises Stemspan II medium.

4. The method of any one of claims 1-3, wherein the expansion medium further comprises dexamethasone.

5. The method of claim 4, wherein the expansion medium comprises dexamethasone at a concentration of 50 nM to 200 nM.

6. The method of any one of claims 1-5, wherein the human CD34⁺ progenitor cells in step (ii) are cultured at an initial cell density from 250,000 to 1,500,000 cells/mL.

7. The method of claim 6, wherein the human CD34⁺ progenitor cells in step (ii) are cultured at an initial cell density of 500,000 cells/ml

8. The method of any of the preceding claims, wherein the human CD34⁺ progenitor cells in step (ii) are expanded for 5 days.

9. The method of any one of claims 1-8, wherein the first differentiation medium comprises SCF at a concentration of 5 ng/ml to 20 ng/ml, IL-3 at a concentration of 0.5 ng/ml to 2 ng/ml, insulin at a concentration of 5 μg/ml to 20 μg/ml; EPO at a concentration of 0.1 U/ml to 6 U/ml and holo-transferrin at a concentration of 100 μg/ml to 400 μg/ml.

10. The method of any one of claims 1-9, wherein the first differentiation medium further comprises IMDM medium.

11. The method of any one of claims 1-10, wherein the first differentiation medium further comprises heparin.

12. The method of claim 11, wherein the first differentiation medium comprises heparin at a concentration of 1.5 U/ml to 6 U/ml.

13. The method of any one of claims 1-12, wherein the first differentiation medium further comprises human blood plasma.

14. The method of claim 13, wherein the first differentiation medium comprises from 1% to 4% human blood plasma.

15. The method of any one of claims 1-13, wherein the first differentiation medium further comprises human serum.

16. The method of claim 15, wherein the first differentiation medium comprises from 1.5% to 6% human serum.

17. The method of any one of claims 1-16, wherein the human CD34⁺ progenitor cells in step (iii) are maintained at a density from 50,000 to 400,000 cells/mL.

18. The method of claim 17, wherein the density of the human CD34⁺ progenitor cells in step (iii) is 100,000 cells/ml

19. The method of any of the preceding claims, wherein the human CD34⁺ progenitor cells in step (iii) are differentiated for 4 days.

20. The method of any one of claims 1-19, wherein the human CD34⁺ progenitor cells in step (iv) are maintained at a density from 100,000 to 600,000 cells/mL.

21. The method of claim 20, wherein the density of the human CD34⁺ progenitor cells in step (iv) is 400,000 cells/ml.

22. The method of any of the preceding claims, wherein the human CD34⁺ progenitor cells in step (iv) are differentiated for 3 days.

23. The method of any one of claims 1-22, wherein the second differentiation medium comprises SCF at a concentration of 5 ng/ml to 20 ng/ml, insulin at a concentration of 5 μg/ml to 20 μg/ml; EPO at a concentration of 0.05 U/ml to 4 U/ml and holo-transferrin at a concentration of 100 μg/ml to 400 μg/ml.

24. The method of any one of claims 1-23, wherein the second differentiation medium further comprises IMDM medium.

25. The method of any one of claims 1-24, wherein the second differentiation medium further comprises heparin.

26. The method of claim 25, wherein the second differentiation medium comprises heparin at a concentration of 1.5 U/ml to 6 U/ml.

27. The method of any one of claims 1-26, wherein the second differentiation medium further comprises human blood plasma.

28. The method of claim 27, wherein the second differentiation medium comprises from 1% to 4% human blood plasma.

29. The method of any one of claims 1-28, wherein the second differentiation medium further comprises human serum.

30. The method of claim 29, wherein the second differentiation medium comprises from 1.5% to 6% human serum.

31. The method of any one of claims 1-30, wherein the human CD34⁺ progenitor cells in step (v) are maintained at a density from 50,000 to 400,000 cells/mL.

32. The method of claim 31, wherein the density of the human CD34⁺ progenitor cells in step (v) is 100,000 cells/ml.

33. The method of any of the preceding claims, wherein the human CD34⁺ progenitor cells in step (v) are differentiated for 4 days.

34. The method of any one of claims 1-33, wherein the third differentiation medium comprises insulin at a concentration of 5 μg/ml to 20 μg/ml; EPO at a concentration of 0.01 U/ml to 0.2 U/ml and holo-transferrin at a concentration of 250 μg/ml to 1000 μg/ml.

35. The method of any one of claims 1-34, wherein the third differentiation medium further comprises IMDM medium.

36. The method of any one of claims 1-35, wherein the third differentiation medium further comprises heparin.

37. The method of claim 36, wherein the third differentiation medium comprises heparin at a concentration of 1.5 U/ml to 6 U/ml.

38. The method of any one of claims 1-37, wherein the third differentiation medium further comprises human blood plasma.

39. The method of claim 38, wherein the third differentiation medium comprises from 1% to 4% human blood plasma.

40. The method of any one of claims 1-39, wherein the third differentiation medium further comprises human serum.

41. The method of claim 40, wherein the third differentiation medium comprises from 1.5% to 6% human serum.

42. The method of any one of claims 1-41, wherein the human CD34⁺ progenitor cells in step (vi) are maintained at a density from 50,000 to 200,000 cells/mL.

43. The method of claim 42, wherein the density of the human CD34⁺ progenitor cells in step (vi) is 100,000 cells/ml.

44. The method of any of the preceding claims, wherein the human CD34⁺ progenitor cells in step (vi) are differentiated for 4 days.

45. The method of any one of claims 1-44, wherein the fourth differentiation medium comprises insulin at a concentration of 5 μg/ml to 20 μg/ml; and holo-transferrin at a concentration of 250 μg/ml to 1000 μg/ml.

46. The method of any one of claims 1-45, wherein the fourth differentiation medium further comprises IMDM medium.

47. The method of any one of claims 1-46, wherein the fourth differentiation medium further comprises heparin.

48. The method of claim 47, wherein the fourth differentiation medium comprises heparin at a concentration of 1.5 U/ml to 6 U/ml.

49. The method of any one of claims 1-48, wherein the fourth differentiation medium further comprises human blood plasma.

50. The method of claim 49, wherein the fourth differentiation medium comprises from 1% to 4% human blood plasma.

51. The method of any one of claims 1-50, wherein the fourth differentiation medium further comprises human serum.

52. The method of claim 51, wherein the fourth differentiation medium comprises from 1.5% to 6% human serum.

53. The method of any one of claims 1-52, wherein the human CD34⁺ progenitor cells in step (vii) are increased to a density from 2,500,000 to 10,000,000 cells/mL.

54. The method of any one of claims 1-53, wherein the human CD34⁺ progenitor cells in step (vii) are increased to a density of 5,000,000 cells/mL.

55. The method of any of the preceding claims, wherein the human CD34⁺ progenitor cells in step (vii) are differentiated for 3 days.

56. A method for producing human red blood cells or enucleated precursors thereof, comprising:
maintaining a plurality of nucleated red blood cell precursors in a maturation medium under maturation conditions that allow for maturation of a plurality of the precursor cells into red blood cells or enucleated precursosrs thereof, wherein:
the level of an erythropoietin (EPO) or an EPO analog in the maturation medium is less than 1 unit/ml;
thereby providing a population comprising red blood cells or enucleated precursors thereof or a combination thereof,
wherein at least 70% of the cells in the population are red blood cells or enucleated precursors thereof or a combination thereof.

57. The method of claim 56, wherein the enucleated precursors are reticulocytes.

58. The method of claim 56 or 57, wherein the level of the erythropoietin (EPO) or the EPO analog in the maturation medium is less than 0.5, 0.3 or 0.1 units/ml.

59. The method of any one of claims 56-58, wherein the erythropoietin (EPO) and/or the EPO analog is absent from the maturation medium.

60. The method of any one of claims 56-59, wherein at least 75%, 80%, 85%, 90%, or 95% of the cells in the population are red blood cells or enucleated precursors thereof, or a combination thereof.

61. A method for producing human red blood cells or enucleated precursors thereof, comprising:
maintaining a plurality of nucleated red blood cell precursors in a maturation medium under maturation conditions that allow for maturation of a plurality of the precursor cells into red blood cells or enucleated precursosrs thereof, wherein:
the level of an erythropoietin (EPO) or an EPO analog in the maturation medium is lower than an amount of an EPO or an EPO analog in a differentiation medium that the cells have been previously cultured in;
thereby providing a population comprising red blood cells or enucleated precursors thereof or a combination thereof,
wherein at least 70% of the cells in the population are red blood cells or enucleated precursors thereof or a combination thereof.

62. The method of claim 61, wherein the level of the erythropoietin (EPO) or the EPO analog in the maturation medium is lower than an amount of an EPO or an EPO analog in differentiation medium IV that the cells have been cultured in prior to the maturation medium.

63. The method of claim 61 or 62, wherein the level of the erythropoietin (EPO) or the EPO analog in the maturation medium is up to 100% lower than an amount of an EPO or an EPO analog in a differentiation medium that the cells have been cultured in prior to the maturation medium.

64. The method of any one of claims 61-63, wherein the level of the erythropoietin (EPO) or the EPO analog in the maturation medium is up to 5%, 10%, 15%, 20%, 30%, 40%, 50%, 60%, 70%, 80%, 90%, 95%, or 99% lower than an amount of an EPO or an EPO analog in a differentiation medium that the cells have been cultured in prior to the maturation medium.

65. The method of any one of claims 61-64, wherein the level of the erythropoietin (EPO) or the EPO analog in the maturation medium is up to 2.0 U/ml lower than an amount of an EPO or an EPO analog in a differentiation medium that the cells have been cultured in prior to the maturation medium.

66. The method of any one of claims 61-65, wherein the level of the erythropoietin (EPO) or the EPO analog in the maturation medium is up to 0.01 U/ml, 0.02 U/ml, 0.03 U/ml, 0.04 U/ml, 0.05 U/ml, 0.06 U/ml, 0.07 U/ml, 0.08 U/ml, 0.09 U/ml, 0.1 U/ml, 0.15 U/ml, 0.2 U/ml, 0.25 U/ml, 0.3 U/ml, 0.4 U/ml, 0.5 U/ml, 0.6 U/ml, 0.7 U/ml, 0.8 U/ml, 1.0 U/ml, or 1.5 U/ml lower than an amount of an EPO or an EPO analog in a differentiation medium that the cells have been cultured in prior to the maturation medium.

67. The method of any one of claims 61-66, wherein at least 75%, 80%, 85%, 90%, or 95% of the cells in the population are red blood cells or enucleated precursors thereof or a combination thereof.

68. The method of any one of claims 61-67, wherein the enucleated precursors are reticulocytes.

69. A fusion protein comprising,
(i) a red blood cell transmembrane protein, and
(ii) a first antigen binding protein that binds to a first epitope of an antigen.

70. The fusion protein of claim 69, wherein the antigen is a toxin or an antigen of a pathogen.

71. The fusion protein of claim 69 or 70, wherein the red blood cell transmembrane protein is glycophorin A (GPA) or Kell.

72. The fusion protein of claim 70 or 71, wherein the toxin is botulinum toxin.

73. The fusion protein of claim 70 or 71, wherein the pathogen is a virus or bacterium.

74. The fusion protein of any one of claims 69-73, wherein the first antigen binding protein is a single-domain antibody.

75. The fusion protein of claim 74, wherein the single-domain antibody is a first single-domain heavy chain antibody (VHH).

76. The fusion protein of any one of claims 69-75, wherein the fusion protein comprises (iii) a second antigen binding protein that binds to a second epitope of the antigen.

77. The fusion protein of claim 76, wherein the second antigen binding protein is a single-domain antibody.

78. The fusion protein of claim 77, wherein the single-domain antibody is a second single-domain heavy chain antibody (VHH).

79. The fusion protein of any one of claims 69-78, wherein the red blood cell transmembrane protein of (i) and the first antigen binding protein of (ii) are fused via a linker.

80. The fusion protein of any one of claims 76-79, wherein the first antigen binding protein of (ii) and the second antigen binding protein of (iii) are fused via a linker.

81. The fusion protein of any one of claims 69-80, wherein the first antigen binding protein of (ii) is fused N-terminal to the red blood cell transmembrane protein of (i).

82. The fusion protein of any one of claims 69-80, where in the first antigen binding protein of (ii) is fused C-terminal to the red blood cell transmembrane protein of (i).

83. The fusion protein of any one of claims 75-82, wherein the fusion protein comprises the structure:
NH₂-[first VHH]-[glycophorin A]-COOH; or
NH₂-[Kell]-[first VHH]-COOH,
wherein “]-[” indicates an optional linker.

84. The fusion protein of any one of claims 78-82, wherein the fusion protein comprises the structure:
NH₂-[second VHH]-[first VHH]-[glycophorin A]-COOH; or
NH₂-[Kell]-[first VHH]-[second VHH]-COOH,
wherein “]-[” indicates an optional linker.

85. The fusion protein of claim 84, wherein the first VHH and the second VHH bind different epitopes of the antigen.

86. The fusion protein of any one of claims 69-85, wherein the fusion protein comprises the amino acid sequence of (SEQ ID NO: 2), (SEQ ID NO: 4), (SEQ ID NO: 6) or (SEQ ID NO: 8).

87. A genetically engineered enucleated blood cell, expressing the fusion protein of any one of claims 69-86 on cell surface.

88. A nucleic acid comprising a nucleic acid sequence that encodes the fusion protein of any one of claims 69-86.

89. A vector comprising the nucleic acid of 88.

90. The vector of 89, wherein the nucleic acid is in operable linkage to a promoter.

91. The vector of claim 89 or 90, wherein the vector is a lentivirus vector or a retrovirus vector.

92. A method of neutralizing a toxin or a pathogen in a subject, the method comprising administering to a subject in need thereof a first dose of the genetically engineered enucleated blood cell of 87.

93. The method of claim 92, wherein the toxin is botulinum toxin.

94. The method of claim 92, wherein the pathogen is a virus or bacterium.

95. The method of any one of claims 92-94, wherein the enucleated blood cell is autologous.

96. The method of any one of claims 92-94, wherein the enucleated blood cell is allogenic.

97. The method of any one of claims 92-96, wherein the subject is infected with Clostridium botulinum.

98. The method of any one of claims 92-97, wherein the subject is administered a second dose of the genetically engineered enucleated blood cell of 87.

99. The method of claim 98, wherein the second dose is administered 5 days or more following the administration of the first dose of the enucleated blood cell.

100. The method of claim 98, wherein the second dose is administered 10 days or more following the administration of the first dose of the enucleated blood cell.

101. The method of claim 98, wherein the second dose is administered 15 days or more following the administration of the first dose of the enucleated blood cell.

102. The method of any one of claims 92-101, wherein the enucleated blood cell is produced using the method of any one of claims 1-68.

103. A method of producing a genetically engineered enucleated blood cell, the method comprising transfecting a CD34⁺ progenitor cell with a vector encoding a protein of interest and culturing the CD34⁺ progenitor cell comprising the vector according to the method of any one of claims 1-68.

104. The method of claim 103, wherein the CD34⁺ progenitor cell is transfected with the vector of any one of claims 89-91.

105. A method of producing a genetically engineered enucleated blood cell, the method comprising:
transfecting a CD34⁺ progenitor cell with a vector encoding a protein of interest, and
administering the CD34⁺ progenitor cell comprising the vector to a subject, wherein the CD34⁺ progenitor cell differentiates into an enucleated blood cell expressing the protein of interest encoded by the vector.

106. The method of claim 105, wherein the vector is a vector of any one of claims 89-91.

107. The method of claim 105 or 106, wherein the subject is infected with or suspected of being infected with a pathogen.

108. The method of claim 107, wherein the pathogen is Clostridium botulinum.


SEQUENCE LISTING (24 sequences)

SEQ ID NO: 1 - DNA, 1308 bp, Artificial Sequence
  Synthetic polynucleotide
atgtatggaaaaataatctttgtattactattgtcagaaattgtgagcatatcagcagaacagaaactgatctctgaagaagacctgcaaggtgttcaagctcaactgcagctcgtggagtcaggtggaggcttggtgcaggttggggggtctctgagactctcctgtgtagtttctggaagcgacatcagtggcattgcgatgggctggtaccgccaggctccagggaagcggcgcgaaatggtcgcagatattttttctggcggtagtacagactatgcaggctccgtgaagggccgattcaccatctccagagacaacgccaagaagacgagctatctgcaaatgaacaacgtgaaacctgaggacaccggagtctactactgtaggctgtacgggagcggtgactactggggccaggggacccaggtcaccgtctcctcagcgcaccacagcgaagaccccactagtgcgatcgctggtggaggcggttcaggcggaggtggctctggcggtggcggttccctgcagggtcagttgcagctcgtggagtccggcggaggcttggtgcaccctggggggtctctgagactctcttgtgcaccctctgccagtctaccatcaacacccttcaaccccttcaacaatatggtgggctggtaccgtcaggctccaggtaaacagcgcgaaatggtcgcaagtattggtctacgaataaactatgcagactccgtgaagggccgattcaccatctccagagacaacgccaagaacacggtggatctgcagatggacagcctgcgacctgaggactcagccacatactactgtcatatagaatacacccactactggggcaaagggaccctggtcaccgtctcctcggaacccaagacaccaaaaccacaaccgttaagtaccactgaggtggcaatgcacacttcaacttcttcttcagtcacaaagagttacatctcatcacagacaaatgatacgcacaaacgggacacatatgcagccactcctagagctcatgaagtttcagaaatttctgttagaactgtttaccctccagaagaggaaaccggagaaagggtacaacttgcccatcatttctctgaaccagagataacactcattatttttggggtgatggctggtgttattggaacgatcctcttaatttcttacggtattcgccgactgataaagaaaagcccatctgatgtaaaacctctcccctcacctgacacagacgtgcctttaagttctgttgaaatagaaaatccagagacaagtgatcaatga

SEQ ID NO: 2 - protein, 435 aa, Artificial Sequence
  Synthetic polypeptide
MYGKIIFVLLLSEIVSISAEQKLISEEDLQGVQAQLQLVESGGGLVQVGGSLRLSCVVSGSDISGIAMGWYRQAPGKRREMVADIFSGGSTDYAGSVKGRFTISRDNAKKTSYLQMNNVKPEDTGVYYCRLYGSGDYWGQGTQVTVSSAHHSEDPTSAIAGGGGSGGGGSGGGGSLQGQLQLVESGGGLVHPGGSLRLSCAPSASLPSTPFNPFNNMVGWYRQAPGKQREMVASIGLRINYADSVKGRFTISRDNAKNTVDLQMDSLRPEDSATYYCHIEYTHYWGKGTLVTVSSEPKTPKPQPLSTTEVAMHTSTSSSVTKSYISSQTNDTHKRDTYAATPRAHEVSEISVRTVYPPEEETGERVQLAHHFSEPEITLIIFGVMAGVIGTILLISYGIRRLIKKSPSDVKPLPSPDTDVPLSSVEIENPETSDQ

SEQ ID NO: 3 - DNA, 1380 bp, Artificial Sequence
  Synthetic polynucleotide
atgtatggaaaaataatctttgtattactattgtcagaaattgtgagcatatcagcagaacagaaactgatctctgaagaagacctgcaaggtgttcaagctcaactgcagctcgtggagtcagggggaggcttggtgcagcctggggggtctctgagactctcctgtgcagcctctggattcactttagatagttatgcaataggctggttccgccaggccccagggaaggagcgtgagggggtcgcatgtattagtgctagtggtagtggcacggactatgtagactccgtgaagggccgattcaccgtctccagagaccaggccaagagcatggtgtttctgcaaatgaacaacatgaaacctgaggacgcagccgtttattactgtgcagcagattatcggccgaggcccctgccgattcaggcgccgtgtacaatgacaggtggcaactactggggccaggggacccaggtcaccgtctcctcagaacccaagacaccaaaaccacaagcgatcgctggtggaggcggttcaggcggaggtggctctggcggtggcggttccctgcagggtcagttgcagctcgtggagtccggtggaggcttggtgcaggctggggggtctctgagactctcctgtgcagcctctatactcacttatgatttggattattattacataggctgggtccgccaggccccagggaaggagcgtgagggggtctcatgtattagtagtactgatggtgccacatactatgcagactccgtgaagggccgattcaccatctccagaaacaacgccaagaacacggtgtatctgcaaatgaacaacctaaaacctgaggacacagccatttattattgtgcagcagcccccctggctgggcgctactgtcccgcctcgcatgagtatggctactggggtcaggggacccaggtcaccgtctcgtcagcgcaccacagcgaagacccctcgtccttaagtaccactgaggtggcaatgcacacttcaacttcttcttcagtcacaaagagttacatctcatcacagacaaatgatacgcacaaacgggacacatatgcagccactcctagagctcatgaagtttcagaaatttctgttagaactgtttaccctccagaagaggaaaccggagaaagggtacaacttgcccatcatttctctgaaccagagataacactcattatttttggggtgatggctggtgttattggaacgatcctcttaatttcttacggtattcgccgactgataaagaaaagcccatctgatgtaaaacctctcccctcacctgacacagacgtgcctttaagttctgttgaaatagaaaatccagagacaagtgatcaatga

SEQ ID NO: 4 - protein, 459 aa, Artificial Sequence
  Synthetic polypeptide
MYGKIIFVLLLSEIVSISAEQKLISEEDLQGVQAQLQLVESGGGLVQPGGSLRLSCAASGFTLDSYAIGWFRQAPGKEREGVACISASGSGTDYVDSVKGRFTVSRDQAKSMVFLQMNNMKPEDAAVYYCAADYRPRPLPIQAPCTMTGGNYWGQGTQVTVSSEPKTPKPQAIAGGGGSGGGGSGGGGSLQGQLQLVESGGGLVQAGGSLRLSCAASILTYDLDYYYIGWVRQAPGKEREGVSCISSTDGATYYADSVKGRFTISRNNAKNTVYLQMNNLKPEDTAIYYCAAAPLAGRYCPASHEYGYWGQGTQVTVSSAHHSEDPSSLSTTEVAMHTSTSSSVTKSYISSQTNDTHKRDTYAATPRAHEVSEISVRTVYPPEEETGERVQLAHHFSEPEITLIIFGVMAGVIGTILLISYGIRRLIKKSPSDVKPLPSPDTDVPLSSVEIENPETSDQ

SEQ ID NO: 5 - DNA, 3054 bp, Artificial Sequence
  Synthetic polynucleotide
atggaaggtggggaccaaagtgaggaagagccgagggaacgcagccaggcaggtggaatgggaactctctggagccaagagagcactccagaagagaggctgcccgtggaagggagcaggccatgggcagtggccaggcgggtgctgacagctatcctgattttgggcctgctcctttgtttttctgtgcttttgttctacaacttccagaactgtggccctcgcccctgtgagacatctgtgtgtttggatctccgggatcattacctggcctctgggaacacaagtgtggccccctgcaccgacttcttcagctttgcctgtggaagggccaaagagaccaataattcttttcaggagcttgccacaaagaacaaaaaccgacttcggagaatactggaggtccagaattcctggcacccaggctctggggaggagaaagccttccagttctacaactcctgcatggatacacttgccattgaagctgcagggactggtcccctcagacaagttattgaggagcttggaggctggcgcatctctggtaaatggacttccttaaactttaaccgaacgctgagacttctgatgagtcagtatggccatttccctttcttcagagcctacctaggacctcatcctgcctctccacacacaccagtcatccagatagaccagccagagtttgatgttcccctcaagcaagatcaagaacagaagatctatgcccagatctttcgggaatacctgacttacctgaatcagctgggaaccttgctgggaggagacccaagcaaggtgcaagaacactcttccttgtcaatctccatcacttcacggctgttccagtttctgaggcccctggagcagcggcgggcacagggcaagctcttccagatggtcactatcgaccagctcaaggaaatggcccccgccatcgactggttgtcctgcttgcaagcgacattcacaccgatgtccctgagcccttctcagtccctcgtggtccatgacgtggaatatttgaaaaacatgtcacaactggtggaggagatgctgctaaagcagagggactttctgcagagccacatgatcttagggctggtggtgaccctttctccagccctggacagtcaattccaggaggcacgcagaaagctcagccagaaactgcgggaactgacagagcaaccacccatgcctgcccgcccacgatggatgaagtgcgtggaggagacaggcacgttcttcgagcccacgctggcggctttgtttgttcgtgaggcctttggcccgagcacccgaagtgctgccatgaaattattcactgcgatccgggatgccctcatcactcgcctcagaaaccttccctggatgaatgaggagacccagaacatggcccaggacaaggttgctcaactgcaggtggagatgggggcttcagaatgggccctgaagccagagctggcccgacaagaatacaacgatatacagcttggatcgagcttcctgcagtctgtcctgagctgtgtccggtccctccgagctagaattgtccagagcttcttgcagcctcacccccaacacaggtggaaggtgtccccttgggacgtcaatgcttactattcggtatctgaccatgtggtagtctttccagctggactcctccaacccccattcttccaccctggctatcccagagccgtgaactttggcgctgctggcagcatcatggcccacgagctgttgcacatcttctaccagctcttactgcctgggggctgcctcgcctgtgacaaccatgccctccaggaagctcacctgtgcctgaagcgccattatgctgcctttccattacctagcagaacctccttcaatgactccctcacattcttagagaatgctgcagacgttggggggctagccatcgcgctgcaggcatacagcaagaggctgttacggcaccatggggagactgtcctgcccagcctggacctcagcccccagcagatcttctttcgaagctatgcccaggtgatgtgtaggaagcccagcccccaggactctcacgacactcacagccctccacacctccgagtccacgggcccctcagcagcaccccagcctttgccaggtatttccgctgtgcacgtggtgctctcttgaacccctccagccgctgccagctctggcaaggtgttcaagctcaactgcagctcgtggagtcaggtggaggcttggtgcaggttggggggtctctgagactctcctgtgtagtttctggaagcgacatcagtggcattgcgatgggctggtaccgccaggctccagggaagcggcgcgaaatggtcgcagatattttttctggcggtagtacagactatgcaggctccgtgaagggccgattcaccatctccagagacaacgccaagaagacgagctatctgcaaatgaacaacgtgaaacctgaggacaccggagtctactactgtaggctgtacgggagcggtgactactggggccaggggacccaggtcaccgtctcctcagcgcaccacagcgaagaccccactagtgcgatcgctggtggaggcggttcaggcggaggtggctctggcggtggcggttccctgcagggtcagttgcagctcgtggagtccggcggaggcttggtgcaccctggggggtctctgagactctcttgtgcaccctctgccagtctaccatcaacacccttcaaccccttcaacaatatggtgggctggtaccgtcaggctccaggtaaacagcgcgaaatggtcgcaagtattggtctacgaataaactatgcagactccgtgaagggccgattcaccatctccagagacaacgccaagaacacggtggatctgcagatggacagcctgcgacctgaggactcagccacatactactgtcatatagaatacacccactactggggcaaagggaccctggtcaccgtctcctcggaacccaagacaccaaaaccacaaccggaacagaaactgatctctgaagaagacctgtaa

SEQ ID NO: 6 - protein, 1017 aa, Artificial Sequence
  Synthetic polypeptide
MEGGDQSEEEPRERSQAGGMGTLWSQESTPEERLPVEGSRPWAVARRVLTAILILGLLLCFSVLLFYNFQNCGPRPCETSVCLDLRDHYLASGNTSVAPCTDFFSFACGRAKETNNSFQELATKNKNRLRRILEVQNSWHPGSGEEKAFQFYNSCMDTLAIEAAGTGPLRQVIEELGGWRISGKWTSLNFNRTLRLLMSQYGHFPFFRAYLGPHPASPHTPVIQIDQPEFDVPLKQDQEQKIYAQIFREYLTYLNQLGTLLGGDPSKVQEHSSLSISITSRLFQFLRPLEQRRAQGKLFQMVTIDQLKEMAPAIDWLSCLQATFTPMSLSPSQSLVVHDVEYLKNMSQLVEEMLLKQRDFLQSHMILGLVVTLSPALDSQFQEARRKLSQKLRELTEQPPMPARPRWMKCVEETGTFFEPTLAALFVREAFGPSTRSAAMKLFTAIRDALITRLRNLPWMNEETQNMAQDKVAQLQVEMGASEWALKPELARQEYNDIQLGSSFLQSVLSCVRSLRARIVQSFLQPHPQHRWKVSPWDVNAYYSVSDHVVVFPAGLLQPPFFHPGYPRAVNFGAAGSIMAHELLHIFYQLLLPGGCLACDNHALQEAHLCLKRHYAAFPLPSRTSFNDSLTFLENAADVGGLAIALQAYSKRLLRHHGETVLPSLDLSPQQIFFRSYAQVMCRKPSPQDSHDTHSPPHLRVHGPLSSTPAFARYFRCARGALLNPSSRCQLWQGVQAQLQLVESGGGLVQVGGSLRLSCVVSGSDISGIAMGWYRQAPGKRREMVADIFSGGSTDYAGSVKGRFTISRDNAKKTSYLQMNNVKPEDTGVYYCRLYGSGDYWGQGTQVTVSSAHHSEDPTSAIAGGGGSGGGGSGGGGSLQGQLQLVESGGGLVHPGGSLRLSCAPSASLPSTPFNPFNNMVGWYRQAPGKQREMVASIGLRINYADSVKGRFTISRDNAKNTVDLQMDSLRPEDSATYYCHIEYTHYWGKGTLVTVSSEPKTPKPQPEQKLISEEDL

SEQ ID NO: 7 - DNA, 3126 bp, Artificial Sequence
  Synthetic polynucleotide
atggaaggtggggaccaaagtgaggaagagccgagggaacgcagccaggcaggtggaatgggaactctctggagccaagagagcactccagaagagaggctgcccgtggaagggagcaggccatgggcagtggccaggcgggtgctgacagctatcctgattttgggcctgctcctttgtttttctgtgcttttgttctacaacttccagaactgtggccctcgcccctgtgagacatctgtgtgtttggatctccgggatcattacctggcctctgggaacacaagtgtggccccctgcaccgacttcttcagctttgcctgtggaagggccaaagagaccaataattcttttcaggagcttgccacaaagaacaaaaaccgacttcggagaatactggaggtccagaattcctggcacccaggctctggggaggagaaagccttccagttctacaactcctgcatggatacacttgccattgaagctgcagggactggtcccctcagacaagttattgaggagcttggaggctggcgcatctctggtaaatggacttccttaaactttaaccgaacgctgagacttctgatgagtcagtatggccatttccctttcttcagagcctacctaggacctcatcctgcctctccacacacaccagtcatccagatagaccagccagagtttgatgttcccctcaagcaagatcaagaacagaagatctatgcccagatctttcgggaatacctgacttacctgaatcagctgggaaccttgctgggaggagacccaagcaaggtgcaagaacactcttccttgtcaatctccatcacttcacggctgttccagtttctgaggcccctggagcagcggcgggcacagggcaagctcttccagatggtcactatcgaccagctcaaggaaatggcccccgccatcgactggttgtcctgcttgcaagcgacattcacaccgatgtccctgagcccttctcagtccctcgtggtccatgacgtggaatatttgaaaaacatgtcacaactggtggaggagatgctgctaaagcagagggactttctgcagagccacatgatcttagggctggtggtgaccctttctccagccctggacagtcaattccaggaggcacgcagaaagctcagccagaaactgcgggaactgacagagcaaccacccatgcctgcccgcccacgatggatgaagtgcgtggaggagacaggcacgttcttcgagcccacgctggcggctttgtttgttcgtgaggcctttggcccgagcacccgaagtgctgccatgaaattattcactgcgatccgggatgccctcatcactcgcctcagaaaccttccctggatgaatgaggagacccagaacatggcccaggacaaggttgctcaactgcaggtggagatgggggcttcagaatgggccctgaagccagagctggcccgacaagaatacaacgatatacagcttggatcgagcttcctgcagtctgtcctgagctgtgtccggtccctccgagctagaattgtccagagcttcttgcagcctcacccccaacacaggtggaaggtgtccccttgggacgtcaatgcttactattcggtatctgaccatgtggtagtctttccagctggactcctccaacccccattcttccaccctggctatcccagagccgtgaactttggcgctgctggcagcatcatggcccacgagctgttgcacatcttctaccagctcttactgcctgggggctgcctcgcctgtgacaaccatgccctccaggaagctcacctgtgcctgaagcgccattatgctgcctttccattacctagcagaacctccttcaatgactccctcacattcttagagaatgctgcagacgttggggggctagccatcgcgctgcaggcatacagcaagaggctgttacggcaccatggggagactgtcctgcccagcctggacctcagcccccagcagatcttctttcgaagctatgcccaggtgatgtgtaggaagcccagcccccaggactctcacgacactcacagccctccacacctccgagtccacgggcccctcagcagcaccccagcctttgccaggtatttccgctgtgcacgtggtgctctcttgaacccctccagccgctgccagctctggcaaggtgttcaagctcaactgcagctcgtggagtcagggggaggcttggtgcagcctggggggtctctgagactctcctgtgcagcctctggattcactttagatagttatgcaataggctggttccgccaggccccagggaaggagcgtgagggggtcgcatgtattagtgctagtggtagtggcacggactatgtagactccgtgaagggccgattcaccgtctccagagaccaggccaagagcatggtgtttctgcaaatgaacaacatgaaacctgaggacgcagccgtttattactgtgcagcagattatcggccgaggcccctgccgattcaggcgccgtgtacaatgacaggtggcaactactggggccaggggacccaggtcaccgtctcctcagaacccaagacaccaaaaccacaagcgatcgctggtggaggcggttcaggcggaggtggctctggcggtggcggttccctgcagggtcagttgcagctcgtggagtccggtggaggcttggtgcaggctggggggtctctgagactctcctgtgcagcctctatactcacttatgatttggattattattacataggctgggtccgccaggccccagggaaggagcgtgagggggtctcatgtattagtagtactgatggtgccacatactatgcagactccgtgaagggccgattcaccatctccagaaacaacgccaagaacacggtgtatctgcaaatgaacaacctaaaacctgaggacacagccatttattattgtgcagcagcccccctggctgggcgctactgtcccgcctcgcatgagtatggctactggggtcaggggacccaggtcaccgtctcgtcagcgcaccacagcgaagacccctcgtccgaacagaaactgatctctgaagaagacctgtaa

SEQ ID NO: 8 - protein, 1041 aa, Artificial Sequence
  Synthetic polypeptide
MEGGDQSEEEPRERSQAGGMGTLWSQESTPEERLPVEGSRPWAVARRVLTAILILGLLLCFSVLLFYNFQNCGPRPCETSVCLDLRDHYLASGNTSVAPCTDFFSFACGRAKETNNSFQELATKNKNRLRRILEVQNSWHPGSGEEKAFQFYNSCMDTLAIEAAGTGPLRQVIEELGGWRISGKWTSLNFNRTLRLLMSQYGHFPFFRAYLGPHPASPHTPVIQIDQPEFDVPLKQDQEQKIYAQIFREYLTYLNQLGTLLGGDPSKVQEHSSLSISITSRLFQFLRPLEQRRAQGKLFQMVTIDQLKEMAPAIDWLSCLQATFTPMSLSPSQSLVVHDVEYLKNMSQLVEEMLLKQRDFLQSHMILGLVVTLSPALDSQFQEARRKLSQKLRELTEQPPMPARPRWMKCVEETGTFFEPTLAALFVREAFGPSTRSAAMKLFTAIRDALITRLRNLPWMNEETQNMAQDKVAQLQVEMGASEWALKPELARQEYNDIQLGSSFLQSVLSCVRSLRARIVQSFLQPHPQHRWKVSPWDVNAYYSVSDHVVVFPAGLLQPPFFHPGYPRAVNFGAAGSIMAHELLHIFYQLLLPGGCLACDNHALQEAHLCLKRHYAAFPLPSRTSFNDSLTFLENAADVGGLAIALQAYSKRLLRHHGETVLPSLDLSPQQIFFRSYAQVMCRKPSPQDSHDTHSPPHLRVHGPLSSTPAFARYFRCARGALLNPSSRCQLWQGVQAQLQLVESGGGLVQPGGSLRLSCAASGFTLDSYAIGWFRQAPGKEREGVACISASGSGTDYVDSVKGRFTVSRDQAKSMVFLQMNNMKPEDAAVYYCAADYRPRPLPIQAPCTMTGGNYWGQGTQVTVSSEPKTPKPQAIAGGGGSGGGGSGGGGSLQGQLQLVESGGGLVQAGGSLRLSCAASILTYDLDYYYIGWVRQAPGKEREGVSCISSTDGATYYADSVKGRFTISRNNAKNTVYLQMNNLKPEDTAIYYCAAAPLAGRYCPASHEYGYWGQGTQVTVSSAHHSEDPSSEQKLISEEDL

SEQ ID NO: 9 - protein, 19 aa, Artificial Sequence
  Synthetic polypeptide
MYGKIIFVLLLSEIVSISA

SEQ ID NO: 10 - protein, 131 aa, Artificial Sequence
  Synthetic polypeptide
LSTTEVAMHTSTSSSVTKSYISSQTNDTHKRDTYAATPRAHEVSEISVRTVYPPEEETGERVQLAHHFSEPEITLIIFGVMAGVIGTILLISYGIRRLIKKSPSDVKPLPSPDTDVPLSSVEIENPETSDQ

SEQ ID NO: 11 - protein, 150 aa, Artificial Sequence
  Synthetic polypeptide
MYGKIIFVLLLSEIVSISALSTTEVAMHTSTSSSVTKSYISSQTNDTHKRDTYAATPRAHEVSEISVRTVYPPEEETGERVQLAHHFSEPEITLIIFGVMAGVIGTILLISYGIRRLIKKSPSDVKPLPSPDTDVPLSSVEIENPETSDQ

SEQ ID NO: 12 - protein, 732 aa, Artificial Sequence
  Synthetic polypeptide
MEGGDQSEEEPRERSQAGGMGTLWSQESTPEERLPVEGSRPWAVARRVLTAILILGLLLCFSVLLFYNFQNCGPRPCETSVCLDLRDHYLASGNTSVAPCTDFFSFACGRAKETNNSFQELATKNKNRLRRILEVQNSWHPGSGEEKAFQFYNSCMDTLAIEAAGTGPLRQVIEELGGWRISGKWTSLNFNRTLRLLMSQYGHFPFFRAYLGPHPASPHTPVIQIDQPEFDVPLKQDQEQKIYAQIFREYLTYLNQLGTLLGGDPSKVQEHSSLSISITSRLFQFLRPLEQRRAQGKLFQMVTIDQLKEMAPAIDWLSCLQATFTPMSLSPSQSLVVHDVEYLKNMSQLVEEMLLKQRDFLQSHMILGLVVTLSPALDSQFQEARRKLSQKLRELTEQPPMPARPRWMKCVEETGTFFEPTLAALFVREAFGPSTRSAAMKLFTAIRDALITRLRNLPWMNEETQNMAQDKVAQLQVEMGASEWALKPELARQEYNDIQLGSSFLQSVLSCVRSLRARIVQSFLQPHPQHRWKVSPWDVNAYYSVSDHVVVFPAGLLQPPFFHPGYPRAVNFGAAGSIMAHELLHIFYQLLLPGGCLACDNHALQEAHLCLKRHYAAFPLPSRTSFNDSLTFLENAADVGGLAIALQAYSKRLLRHHGETVLPSLDLSPQQIFFRSYAQVMCRKPSPQDSHDTHSPPHLRVHGPLSSTPAFARYFRCARGALLNPSSRCQLW

SEQ ID NO: 13 - protein, 275 aa, Artificial Sequence
  Synthetic polypeptide
QGVQAQLQLVESGGGLVQVGGSLRLSCVVSGSDISGIAMGWYRQAPGKRREMVADIFSGGSTDYAGSVKGRFTISRDNAKKTSYLQMNNVKPEDTGVYYCRLYGSGDYWGQGTQVTVSSAHHSEDPTSAIAGGGGSGGGGSGGGGSLQGQLQLVESGGGLVHPGGSLRLSCAPSASLPSTPFNPFNNMVGWYRQAPGKQREMVASIGLRINYADSVKGRFTISRDNAKNTVDLQMDSLRPEDSATYYCHIEYTHYWGKGTLVTVSSEPKTPKPQP

SEQ ID NO: 14 - protein, 299 aa, Artificial Sequence
  Synthetic polypeptide
QGVQAQLQLVESGGGLVQPGGSLRLSCAASGFTLDSYAIGWFRQAPGKEREGVACISASGSGTDYVDSVKGRFTVSRDQAKSMVFLQMNNMKPEDAAVYYCAADYRPRPLPIQAPCTMTGGNYWGQGTQVTVSSEPKTPKPQAIAGGGGSGGGGSGGGGSLQGQLQLVESGGGLVQAGGSLRLSCAASILTYDLDYYYIGWVRQAPGKEREGVSCISSTDGATYYADSVKGRFTISRNNAKNTVYLQMNNLKPEDTAIYYCAAAPLAGRYCPASHEYGYWGQGTQVTVSSAHHSEDPSS

SEQ ID NO: 15 - protein, 108 aa, Artificial Sequence
  Synthetic polypeptide
SGGGLVQVGGSLRLSCVVSGSDISGIAMGWYRQAPGKRREMVADIFSGGSTDYAGSVKGRFTISRDNAKKTSYLQMNNVKPEDTGVYYCRLYGSGDYWGQGTQVTVSS

SEQ ID NO: 16 - protein, 111 aa, Artificial Sequence
  Synthetic polypeptide
SGGGLVHPGGSLRLSCAPSASLPSTPFNPFNNMVGWYRQAPGKQREMVASIGLRINYADSVKGRFTISRDNAKNTVDLQMDSLRPEDSATYYCHIEYTHYWGKGTLVTVSS

SEQ ID NO: 17 - protein, 123 aa, Artificial Sequence
  Synthetic polypeptide
SGGGLVQPGGSLRLSCAASGFTLDSYAIGWFRQAPGKEREGVACISASGSGTDYVDSVKGRFTVSRDQAKSMVFLQMNNMKPEDAAVYYCAADYRPRPLPIQAPCTMTGGNYWGQGTQVTVSS

SEQ ID NO: 18 - protein, 121 aa, Artificial Sequence
  Synthetic polypeptide
SGGGLVQAGGSLRLSCAASILTYDLDYYYIGWVRQAPGKEREGVSCISSTDGATYYADSVKGRFTISRNNAKNTVYLQMNNLKPEDTAIYYCAAAPLAGRYCPASHEYGYWGQGTQVTVSS

SEQ ID NO: 19 - protein, 11 aa, Artificial Sequence
  Synthetic polypeptide
QGVQAQLQLVE

SEQ ID NO: 20 - protein, 36 aa, Artificial Sequence
  Synthetic polypeptide
AHHSEDPTSAIAGGGGSGGGGSGGGGSLQGQLQLVE

SEQ ID NO: 21 - protein, 9 aa, Artificial Sequence
  Synthetic polypeptide
EPKTPKPQP

SEQ ID NO: 22 - protein, 35 aa, Artificial Sequence
  Synthetic polypeptide
EPKTPKPQAIAGGGGSGGGGSGGGGSLQGQLQLVE

SEQ ID NO: 23 - protein, 9 aa, Artificial Sequence
  Synthetic polypeptide
AHHSEDPSS

SEQ ID NO: 24 - protein, 19 aa, Artificial Sequence
  Synthetic polypeptide
MYGKIIFVLLLSEIVSISA